(19) United States
(12) Patent Application Publication
(10) Pub. No.: US 2025/0197822 A1
(43) Pub. Date: Jun. 19, 2025

(54) NOVEL OMNI-59, 61, 67, 76, 79, 80, 81 AND 82 CRISPR NUCLEASES

(71) Applicant: EmendoBio Inc., Wilmington, DE (US)

(72) Inventors: Lior Izhar, Tel Aviv (IL); Liat Rockah, Bishon LeZion (IL); Nadav Marbach Bar, Rehovot (IL); Nurit Meron, Ramat Gan (IL)

(73) Assignee: EmendoBio Inc., Wilmington, DE (US)

(21) Appl. No.: 18/419,697

(22) Filed: Jan. 23, 2024

Related U.S. Application Data
(63) Continuation of application No. 17/905,617, filed on Sep. 2, 2022, now Pat. No. 11,946,077.
(60) Provisional application No. 63/122,564, filed on Dec. 8, 2020.
(60) Provisional application No. 63/034,610, filed on Jun. 4, 2020.

(51) Int. Cl. C12N 9/22

(52) U.S. Cl. CPC C12N 9/22; C12N 2310/20

(57) ABSTRACT

The present invention provides a non-naturally occurring composition comprising a CRISPR nuclease comprising a sequence having at least 90% identity to the amino acid sequence selected from the group consisting of SEQ ID NOs: 1, 2, 3, 4, 6, 7, or 8, or a nucleic acid molecule comprising a sequence encoding the CRISPR nuclease.

### CROSS-REFERENCE TO RELATED APPLICATIONS

This application is a continuation of U.S. application Ser. No. 17/905,617, filed Sep. 2, 2022, which is a § 371 national stage of PCT International Application No. PCT/US2021/035928, filed Jun. 4, 2021, claiming the benefit of U.S. Provisional Application Nos. 63/122,564, filed Dec. 8, 2020, and 63/034,610, filed Jun. 4, 2020, the contents of each of which are hereby incorporated by reference into the application.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “18419697_OMNI59_0035US02.xml”, which is 415,839 bytes in size, and which was created on Mar. 10, 2025 in the IBM-PC machine format, having an operating system compatibility with MS-Windows, which is contained in the text file filed Mar. 10, 2025 as part of the parent application.

### FIELD OF THE INVENTION

The present invention is directed to, inter alia, composition and methods for genome editing.

### BACKGROUND OF THE INVENTION

The Clustered Regularly Interspaced Short Palindromic Repeat (CRISPR) systems of bacterial and archaeal adaptive immunity show extreme diversity of protein composition and genomic loci architecture. The CRISPR systems have become important tools for research and genome engineering. Nevertheless, many details of CRISPR systems have not been determined and the applicability of CRISPR nucleases may be limited by sequence specificity requirements, expression, or delivery challenges. Different CRISPR nucleases have diverse characteristics such as: size, PAM site, on target activity, specificity, cleavage pattern (e.g. blunt, staggered ends), and prominent pattern of indel formation following cleavage. Different sets of characteristics may be useful for different applications. For example, some CRISPR nucleases may be able to target particular genomic loci that other CRISPR nucleases cannot due to limitations of the PAM site. In addition, some CRISPR nucleases currently in use exhibit pre-immunity, which may limit in vivo applicability. See Charlesworth et al., Nature Medicine (2019) and Wagner et al., Nature Medicine (2019). Accordingly, discovery, engineering, and improvement of novel CRISPR nucleases is of importance.

### SUMMARY OF THE INVENTION

Disclosed herein are compositions and methods that may be utilized for genomic engineering, epigenomic engineering, genome targeting, genome editing of cells, and/or in vitro diagnostics.

The disclosed compositions may be utilized for modifying genomic DNA sequences. As used herein, genomic DNA refers to linear and/or chromosomal DNA and/or plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of a DNA sequence at the target site(s) in a genome.

Accordingly, in some embodiments, the compositions comprise a Clustered Regularly Interspaced Short Palindromic Repeat (CRISPR) nucleases. In some embodiments, the CRISPR nuclease is a CRISPR-associated protein.

In some embodiments, the compositions comprise a Clustered Regularly Interspaced Short Palindromic Repeats (CRISPR) nuclease having 100%, 99%, 98%, 97%, 96%, 95%, 94%, 93%, 92%, 91%, 90%, 85% identity to CRISPR nucleases derived from Acetobacterium sp. KB-1, Alistipes sp. An54, Bartonella apis, Blastopirellula marina, Bryobacter aggregatus MPL3, Algoriphagus marinus, Butyrivibrio sp. AC2005, bacterium LF-3, Aliiarcobacter faecis, Caviibacter abscessus, Arcobacter sp. SM1702, Arcobacter mytili, Arcobacter thereius, Carnobacterium funditum, Peptoniphilus obesi ph1, Camobacterium iners, Lactobacillus allii, Bacteroides coagulans, Butyrivibrio sp. NC3005, Clostridium sp. AF02-29 or Algoriphagus antarcticus. Each possibility represents a separate embodiment.

### OMNI CRISPR Nucleases

Embodiments of the present invention provide for CRISPR nucleases designated as an “OMNI” nuclease as provided in Tables 1a-1b.

This invention provides a method of modifying a nucleotide sequence at a target site in the genome of a mammalian cell comprising introducing into the cell (i) a composition comprising a CRISPR nuclease having at least 95% identity to an amino acid sequence selected from the group consisting of SEQ ID NOs: 1-8 or a nucleic acid molecule comprising a sequence encoding a CRISPR nuclease which sequence has at least 95% identity to a nucleic acid sequence selected from the group consisting of SEQ ID NOs: 9-24 and (ii) a DNA-targeting RNA molecule, or a DNA polynucleotide encoding a DNA-targeting RNA molecule, comprising a nucleotide sequence that is complementary to a sequence in the target DNA.

This invention also provides a non-naturally occurring composition comprising a CRISPR associated system comprising:
a) one or more RNA molecules comprising a guide sequence portion linked to a direct repeat sequence, wherein the guide sequence is capable of hybridizing with a target sequence, or one or more nucleotide sequences encoding the one or more RNA molecules; and
b) a CRISPR nuclease comprising an amino acid sequence having at least 95% identity to the amino acid sequence selected from the group consisting of SEQ ID NOs: 1-8 or a nucleic acid molecule comprising a sequence encoding the CRISPR nuclease; and
wherein the one or more RNA molecules hybridize to the target sequence, wherein the target sequence is adjacent to the 3′ end of a complimentary sequence of a Protospacer Adjacent Motif (PAM), and the one or more RNA molecules form a complex with the RNA-guided nuclease.

This invention also provides a non-naturally occurring composition comprising:
a) a CRISPR nuclease comprising a sequence having at least 95% identity to the amino acid sequence selected from the group consisting of SEQ ID NOs: 1-8 or a nucleic acid molecule comprising a sequence encoding the CRISPR nuclease; and
b) one or more RNA molecules, or one or more DNA polynucleotide encoding the one or more RNA molecules, comprising at least one of:
   i) a nuclease-binding RNA nucleotide sequence capable of interacting with/binding to the CRISPR nuclease; and
   ii) a DNA-targeting RNA nucleotide sequence comprising a sequence complementary to a sequence in a target DNA sequence,
wherein the CRISPR nuclease is capable of complexing with the one or more RNA molecules to form a complex capable of hybridizing with the target DNA sequence.

### BRIEF DESCRIPTION OF THE DRAWINGS

FIGS. 1A-1C: The predicted secondary structure of a single guide RNA (sgRNA) (crRNA-tracrRNA) from Novosphingobium sp. SYSU G00007 (OMNI-79) comprising a guide sequence portion and a sequence selected from the group consisting of SEQ ID Nos: 63-90: The crRNA and tracrRNA portions of the sgRNA are noted. FIG. 1A: The native pre-mature crRNA-tracrRNA duplex. FIG. 1B: Example of V1 sgRNA design with the duplex shortening (indicated by triangles in A) compared with the native. FIG. 1C: Example of V2 sgRNA design with the duplex shortening (indicated by triangles in A) compared with the native.

FIGS. 2A-2H: In-vitro PAM Depletion by TXTL results for OMNI nucleases. The PAM logo is a schematic representation of the ratio of the depleted site. A condensed 4N window library of all possible PAM locations along an 8 bp sequence for each OMNI nuclease in a cell-free in vitro TXTL system is shown. Sequence motifs generated for in vitro PAM sites are based on depletion assay results. Activity estimated based on the average of the two most depleted sequences and was calculated as: 1−Depletion score. In vitro PAM depletion results for OMNI-59 sgRNA v1 and v2 (FIG. 2A); OMNI-61 sgRNA v1, and v2 (FIG. 2B); OMNI-67 sgRNA v1, and v2 (FIG. 2C); OMNI-76 sgRNA v1 and v2 (FIG. 2D); OMNI-79 sgRNA v1 and v2 (FIG. 2E); OMNI-80 sgRNA v1 and v2 (FIG. 2F); OMNI-81 sgRNA v1 and v2 (FIG. 2G); and OMNI-82 sgRNA v1 and v2 (FIG. 2H) are depicted.

FIG. 3A: In-vivo PAM enrichment by DNA transfection results for OMNI nucleases. Hek293 cells with a PAM library of 6N after the T2 site were transfected with an OMNI-79 nuclease and a T2 sgRNA. Cells were harvested at Day 6 and probed on a western blot using an antibody against an HA-tag to confirm expression of the OMNI-79 nuclease in the mammalian system. Lane 1 is a lysate containing SpCas9-HA, Lane 2 is a lysate containing OMNI-79-HA, and Lane 3 is a non-transfected lysate. FIG. 3B: NGS analysis: NGS analysis was used to select indel containing sequences. Nucleotide frequencies for every position of the PAM in the enriched population is described for both V1 and V2 versions of the sgRNA.

FIGS. 4A-4B: OMNI79 activity with gRNA variation: FIG. 4A: The predicted secondary structure of a single-guide RNA (sgRNA) (crRNA-tracrRNA) scaffolds from Novosphingobium sp. SYSU G00007 (OMNI-79) and two other sgRNA scaffolds, SpSpCAP1 and SpSaNXO2, are presented. FIG. 4B: HeLa cells were transfected with gRNA molecules composed of spacers directed to CXCR4, TRAC, or ELANE genes, and either an OMNI-79 native scaffold, a SpSpCAP1 scaffold, or a SpSaNXO2 scaffold. Editing activity of OMNI-79 nuclease with each RNA molecule was determined using amplicon-based next-generation sequencing (NGS) measuring indel frequency.

FIGS. 5A-5B: OMNI79 activity via AAV delivery. FIG. 5A: HeLa cells were infected using AAV particles bearing OMNI-79 CRISPR nuclease under a CMV promoter and a gRNA molecule targeting CXCR4, ELANE, or A1AT genes and under a U6 promoter. Editing activity was determined using amplicon-based next-generation sequencing (NGS) measuring indel frequency. FIG. 5B: HepG2 cells were infected with OMNI-79 CRIPSR nuclease and a A1AT-targeting gRNA molecule with or without bortezomib (BTZ). Editing activity was determined using amplicon-based next-generation sequencing (NGS) measuring indel frequency.

FIG. 6: OMNI-79 CRIPSR nuclease spacer optimization. OMNI-79 protein was purified and a RNP complex was assembled using a gRNA molecule targeting ELANE g35 with a spacer length of 20 nt, 21 nt, 22 nt, 23 nt, 24 nt, 25 nt, or 26 nt. All gRNA molecules are synthetic with 2′-O-methyl 3′-phosphorothioate modifications of the first and last three (3) nucleotides. The gRNA molecule with 25-nucleotide long spacer was also synthesized as a version without modifications. Activity of OMNI-79 CRISPR nuclease using the different gRNA molecules was tested in U2OS cells (FIG. 6A) or in an in-vitro activity assay (FIG. 6B).

### DETAILED DESCRIPTION

According to embodiments of the present invention, there is provided a non-naturally occurring composition comprising a CRISPR nuclease comprising a sequence having at least 90% identity to the amino acid sequence selected from the group consisting of SEQ ID NOs: 1-8, or a nucleic acid molecule comprising a sequence encoding the CRISPR nuclease.

In some embodiments, the composition further comprises one or more RNA molecules, or a DNA polynucleotide encoding any one of the one or more RNA molecules, wherein the one or more RNA molecules and the CRISPR nuclease do not naturally occur together and the one or more RNA molecules are configured to form a complex with the CRISPR nuclease and/or target the complex to a target site. In some embodiments, the target site is genomic DNA target site in a eukaryotic cell. In some embodiments, the RNA molecule or molecules are modified to have a 2′-O-methyl 3′-phosphorothioate at its 3′ end, 5′ end, or at both ends.

In some embodiments, the CRISPR nuclease comprises a sequence having at least 90% identity to the amino acid sequence set forth in SEQ ID NO: 5, and at least one RNA molecule comprises a sequence selected from the group consisting of SEQ ID NOs: 63-90.

In some embodiments, the CRISPR nuclease comprises a sequence having at least 90% identity to the amino acid sequence set forth in SEQ ID NO: 5 and at least one RNA molecule is a CRISPR RNA (crRNA) molecule comprising a guide sequence portion and a sequence selected from the group consisting of SEQ ID NOs: 63, 64, 71-73, and 81-83.

In some embodiments, the composition further comprises a transactivating CRISPR RNA (tracrRNA) molecule comprising a sequence set forth in the group consisting of SEQ ID NOs: 65-69, 74-79, and 84-89.

In some embodiments, the CRISPR nuclease comprises a sequence having at least 90% identity to the amino acid sequence set forth in SEQ ID NO: 5 and at least one RNA molecule is a single-guide RNA (sgRNA) molecule comprising a guide sequence portion and a sequence selected from the group consisting of SEQ ID NOs: 63-90.

In some embodiments, the guide sequence portion is 25 or 26 nucleotides in length.

In some embodiments, the CRISPR nuclease comprises a sequence having at least 90% identity to the amino acid sequence of SEQ ID NO: 5, and wherein the CRISPR nuclease is a nickase formed by an amino acid substitution at D8, E502, H735 or D738 of SEQ ID NO: 5.

In some embodiments, the CRISPR nuclease comprises a sequence having at least 90% identity to the amino acid sequence of SEQ ID NO: 5, and wherein the CRISPR nuclease is a nickase formed by an amino acid substitution at D586, H587 or N610 of SEQ ID NO: 5.

In some embodiments, the CRISPR nuclease comprises a sequence having at least 90% identity to the amino acid sequence of SEQ ID NO: 5, and wherein the CRISPR nuclease is a catalytically inactive nuclease formed by an amino acid substitution at D8, E502, H735 or D738 of SEQ ID NO: 5, and an amino acid substitution at D586, H587 or N610 of SEQ ID NO: 5.

In some embodiments, the CRISPR nuclease comprises a sequence having at least 90% identity to the amino acid sequence of SEQ ID NO: 5, and the CRISPR nuclease comprises a Domain A comprising a sequence having at least 90% identity to the amino acid sequence of at least 90% sequence identity to amino acids 1 to 40 of SEQ ID NO: 5.

In some embodiments, the CRISPR nuclease comprises a sequence having at least 90% identity to the amino acid sequence of SEQ ID NO: 5, and the CRISPR nuclease comprises a Domain B comprising a sequence having at least 90% identity to the amino acid sequence of at least 90% sequence identity to amino acids 41 to 76 of SEQ ID NO. 5.

In some embodiments, the CRISPR nuclease comprises a sequence having at least 90% identity to the amino acid sequence of SEQ ID NO: 5, and the CRISPR nuclease comprises a Domain C comprising a sequence having at least 90% identity to the amino acid sequence of at least 90% sequence identity to amino acids 77 to 228 of SEQ ID NO: 5.

In some embodiments, the CRISPR nuclease comprises a sequence having at least 90% identity to the amino acid sequence of SEQ ID NO: 5, and the CRISPR nuclease comprises a Domain D comprising a sequence having at least 90% identity to the amino acid sequence of at least 90% sequence identity to amino acids 229 to 446 of SEQ ID NO: 5.

In some embodiments, the CRISPR nuclease comprises a sequence having at least 90% identity to the amino acid sequence of SEQ ID NO: 5, and the CRISPR nuclease comprises a Domain E comprising a sequence having at least 90% identity to the amino acid sequence of at least 90% sequence identity to amino acids 447 to 507 of SEQ ID NO: 5.

In some embodiments, the CRISPR nuclease comprises a sequence having at least 90% identity to the amino acid sequence of SEQ ID NO: 5, and the CRISPR nuclease comprises a Domain F comprising a sequence having at least 90% identity to the amino acid sequence of at least 90% sequence identity to amino acids 539 to 648 of SEQ ID NO: 5.

In some embodiments, the CRISPR nuclease comprises a sequence having at least 90% identity to the amino acid sequence of SEQ ID NO: 5, and the CRISPR nuclease comprises a Domain G comprising a sequence having at least 90% identity to the amino acid sequence of at least 90% sequence identity to amino acids 655 to 822 of SEQ ID NO: 5

In some embodiments, the CRISPR nuclease comprises a sequence having at least 90% identity to the amino acid sequence of SEQ ID NO: 5, and the CRISPR nuclease comprises a Domain H comprising a sequence having at least 90% identity to the amino acid sequence of at least 90% sequence identity to amino acids 823 to 921 of SEQ ID NO: 5.

In some embodiments, the CRISPR nuclease comprises a sequence having at least 90% identity to the amino acid sequence of SEQ ID NO: 5, and the CRISPR nuclease comprises a Domain I comprising a sequence having at least 90% identity to the amino acid sequence of at least 90% sequence identity to amino acids 922 to 1062 of SEQ ID NO: 5.

In some embodiments, the CRISPR nuclease comprises a sequence having at least 90% identity to the amino acid sequence set forth in SEQ ID NO: 1, and at least one RNA molecule comprises a sequence selected from the group consisting of SEQ ID NOs: 25-29.

In some embodiments, the CRISPR nuclease comprises a sequence having at least 90% identity to the amino acid sequence set forth in SEQ ID NO: 1 and at least one RNA molecule is a CRISPR RNA (crRNA) molecule comprising a guide sequence portion and a sequence set forth in SEQ ID NO: 25.

In some embodiments, the composition further comprises a transactivating CRISPR RNA (tracrRNA) molecule comprising a sequence set forth in the group consisting of SEQ ID NOs: 26-28.

In some embodiments, the CRISPR nuclease comprises a sequence having at least 90% identity to the amino acid sequence set forth in SEQ ID NO: 1 and at least one RNA molecule is a single-guide RNA (sgRNA) molecule comprising a guide sequence portion and a sequence selected from the group consisting of SEQ ID NOs: 25-29.

In some embodiments, the CRISPR nuclease comprises a sequence having at least 90% identity to the amino acid sequence of SEQ ID NO: 1, and wherein the CRISPR nuclease is a nickase formed by an amino acid substitution at D24, E557, H785 or D788 of SEQ ID NO: 1.

In some embodiments, the CRISPR nuclease comprises a sequence having at least 90% identity to the amino acid sequence of SEQ ID NO: 1, and wherein the CRISPR nuclease is a nickase formed by an amino acid substitution at E644, H645 or N668 of SEQ ID NO: 1.

In some embodiments, the CRISPR nuclease comprises a sequence having at least 90% identity to the amino acid sequence of SEQ ID NO: 1, and wherein the CRISPR nuclease is a catalytically inactive nuclease formed by an amino acid substitution at D24, E557, H785 or D788 of SEQ ID NO: 1, and an amino acid substitution at E644, H645 or N668 of SEQ ID NO: 1.

In some embodiments, the CRISPR nuclease comprises a sequence having at least 90% identity to the amino acid sequence set forth in SEQ ID NO: 2, and at least one RNA molecule comprises a sequence selected from the group consisting of SEQ ID NOs: 30-39.

In some embodiments, the CRISPR nuclease comprises a sequence having at least 90% identity to the amino acid sequence set forth in SEQ ID NO: 2 and at least one RNA molecule is a CRISPR RNA (crRNA) molecule comprising a guide sequence portion and a sequence selected from the group consisting of SEQ ID NOs: 30-32.

In some embodiments, the composition further comprises a transactivating CRISPR RNA (tracrRNA) molecule comprising a sequence set forth in the group consisting of SEQ ID NOs: 33-38.

In some embodiments, the CRISPR nuclease comprises a sequence having at least 90% identity to the amino acid sequence set forth in SEQ ID NO: 2 and at least one RNA molecule is a single-guide RNA (sgRNA) molecule comprising a guide sequence portion and a sequence selected from the group consisting of SEQ ID NOs: 30-39.

In some embodiments, the CRISPR nuclease comprises a sequence having at least 90% identity to the amino acid sequence of SEQ ID NO: 2, and wherein the CRISPR nuclease is a nickase formed by an amino acid substitution at D19, E528, H750 or D753 of SEQ ID NO: 2.

In some embodiments, the CRISPR nuclease comprises a sequence having at least 90% identity to the amino acid sequence of SEQ ID NO: 2, and wherein the CRISPR nuclease is a nickase formed by an amino acid substitution at D609, H610 or N633 of SEQ ID NO: 2.

In some embodiments, the CRISPR nuclease comprises a sequence having at least 90% identity to the amino acid sequence of SEQ ID NO: 2, and wherein the CRISPR nuclease is a catalytically inactive nuclease formed by an amino acid substitution at D19, E528, H750 or D753 of SEQ ID NO: 2, and an amino acid substitution at D609, H610 or N633 of SEQ ID NO: 2.

In some embodiments, the CRISPR nuclease comprises a sequence having at least 90% identity to the amino acid sequence set forth in SEQ ID NO: 3, and at least one RNA molecule comprises a sequence selected from the group consisting of SEQ ID NOs: 40-52

In some embodiments, the CRISPR nuclease comprises a sequence having at least 90% identity to the amino acid sequence set forth in SEQ ID NO: 3 and at least one RNA molecule is a CRISPR RNA (crRNA) molecule comprising a guide sequence portion and a sequence selected from the group consisting of SEQ ID NOs: 40-43.

In some embodiments, the composition further comprises a transactivating CRISPR RNA (tracrRNA) molecule comprising a sequence set forth in the group consisting of SEQ ID NOs: 44-51.

In some embodiments, the CRISPR nuclease comprises a sequence having at least 90% identity to the amino acid sequence set forth in SEQ ID NO: 3 and at least one RNA molecule is a single-guide RNA (sgRNA) molecule comprising a guide sequence portion and a sequence selected from the group consisting of SEQ ID NOs: 40-52.

In some embodiments, the CRISPR nuclease comprises a sequence having at least 90% identity to the amino acid sequence of SEQ ID NO: 3, and wherein the CRISPR nuclease is a nickase formed by an amino acid substitution at D8, E503, H729 or D732 of SEQ ID NO: 3.

In some embodiments, the CRISPR nuclease comprises a sequence having at least 90% identity to the amino acid sequence of SEQ ID NO: 3, and wherein the CRISPR nuclease is a nickase formed by an amino acid substitution at E584, H585 or N607 of SEQ ID NO: 3.

In some embodiments, the CRISPR nuclease comprises a sequence having at least 90% identity to the amino acid sequence of SEQ ID NO: 3, and wherein the CRISPR nuclease is a catalytically inactive nuclease formed by an amino acid substitution at D8, E503, H729 or D732 of SEQ ID NO: 1, and an amino acid substitution at E584, H585 or N607 of SEQ ID NO: 3.

In some embodiments, the CRISPR nuclease comprises a sequence having at least 90% identity to the amino acid sequence set forth in SEQ ID NO: 4, and at least one RNA molecule comprises a sequence selected from the group consisting of SEQ ID NOs: 53-62.

In some embodiments, the CRISPR nuclease comprises a sequence having at least 90% identity to the amino acid sequence set forth in SEQ ID NO: 4 and at least one RNA molecule is a CRISPR RNA (crRNA) molecule comprising a guide sequence portion and a sequence selected from the group consisting of SEQ ID NOs: 53-55.

In some embodiments, the composition further comprises a transactivating CRISPR RNA (tracrRNA) molecule comprising a sequence set forth in the group consisting of SEQ ID NOs: 56-61.

In some embodiments, the CRISPR nuclease comprises a sequence having at least 90% identity to the amino acid sequence set forth in SEQ ID NO: 4 and at least one RNA molecule is a single-guide RNA (sgRNA) molecule comprising a guide sequence portion and a sequence selected from the group consisting of SEQ ID NOs: 53-62.

In some embodiments, the CRISPR nuclease comprises a sequence having at least 90% identity to the amino acid sequence of SEQ ID NO: 4, and wherein the CRISPR nuclease is a nickase formed by an amino acid substitution at D12, E543, H770 or D773 of SEQ ID NO: 4.

In some embodiments, the CRISPR nuclease comprises a sequence having at least 90% identity to the amino acid sequence of SEQ ID NO: 4, and wherein the CRISPR nuclease is a nickase formed by an amino acid substitution at E630, H631 or N654 of SEQ ID NO: 4.

In some embodiments, the CRISPR nuclease comprises a sequence having at least 90% identity to the amino acid sequence of SEQ ID NO: 4, and wherein the CRISPR nuclease is a catalytically inactive nuclease formed by an amino acid substitution at D12, E543, H770 or D773 of SEQ ID NO: 4, and an amino acid substitution at E630, H631 or N654 of SEQ ID NO: 4.

In some embodiments, the CRISPR nuclease comprises a sequence having at least 90% identity to the amino acid sequence set forth in SEQ ID NO: 6, and at least one RNA molecule comprises a sequence selected from the group consisting of SEQ ID NOs: 91-98.

In some embodiments, the CRISPR nuclease comprises a sequence having at least 90% identity to the amino acid sequence set forth in SEQ ID NO: 6 and at least one RNA molecule is a CRISPR RNA (crRNA) molecule comprising a guide sequence portion and a sequence selected from the group consisting of SEQ ID NOs: 91 and 92.

In some embodiments, the composition further comprises a transactivating CRISPR RNA (tracrRNA) molecule comprising a sequence set forth in the group consisting of SEQ ID NOs: 93-97.

In some embodiments, the CRISPR nuclease comprises a sequence having at least 90% identity to the amino acid sequence set forth in SEQ ID NO: 6 and at least one RNA molecule is a single-guide RNA (sgRNA) molecule comprising a guide sequence portion and a sequence selected from the group consisting of SEQ ID NOs: 91-98.

In some embodiments, the CRISPR nuclease comprises a sequence having at least 90% identity to the amino acid sequence of SEQ ID NO: 6, and wherein the CRISPR nuclease is a nickase formed by an amino acid substitution at D8, E523, H757 or D760 of SEQ ID NO: 6.

In some embodiments, the CRISPR nuclease comprises a sequence having at least 90% identity to the amino acid sequence of SEQ ID NO: 6, and wherein the CRISPR nuclease is a nickase formed by an amino acid substitution at D607, H608 or N631 of SEQ ID NO: 6.

In some embodiments, the CRISPR nuclease comprises a sequence having at least 90% identity to the amino acid sequence of SEQ ID NO: 6, and wherein the CRISPR nuclease is a catalytically inactive nuclease formed by an amino acid substitution at D8, E523, H757 or D760 of SEQ ID NO: 6, and an amino acid substitution at D607, H608 or N631 of SEQ ID NO: 6.

In some embodiments, the CRISPR nuclease comprises a sequence having at least 90% identity to the amino acid sequence set forth in SEQ ID NO: 7, and at least one RNA molecule comprises a sequence selected from the group consisting of SEQ ID NOs: 99-108.

In some embodiments, the CRISPR nuclease comprises a sequence having at least 90% identity to the amino acid sequence set forth in SEQ ID NO: 7 and at least one RNA molecule is a CRISPR RNA (crRNA) molecule comprising a guide sequence portion and a sequence selected from the group consisting of SEQ ID NOs: 99-101.

In some embodiments, the composition further comprises a transactivating CRISPR RNA (tracrRNA) molecule comprising a sequence set forth in the group consisting of SEQ ID NOs: 102-107.

In some embodiments, the CRISPR nuclease comprises a sequence having at least 90% identity to the amino acid sequence set forth in SEQ ID NO: 7 and at least one RNA molecule is a single-guide RNA (sgRNA) molecule comprising a guide sequence portion and a sequence selected from the group consisting of SEQ ID NOs: 99-108.

In some embodiments, the CRISPR nuclease comprises a sequence having at least 90% identity to the amino acid sequence of SEQ ID NO: 7, and wherein the CRISPR nuclease is a nickase formed by an amino acid substitution at D12, E527, H756 or D759 of SEQ ID NO: 7.

In some embodiments, the CRISPR nuclease comprises a sequence having at least 90% identity to the amino acid sequence of SEQ ID NO: 7, and wherein the CRISPR nuclease is a nickase formed by an amino acid substitution at E615, H616 or N639 of SEQ ID NO: 7.

In some embodiments, the CRISPR nuclease comprises a sequence having at least 90% identity to the amino acid sequence of SEQ ID NO: 7, and wherein the CRISPR nuclease is a catalytically inactive nuclease formed by an amino acid substitution at D12, E527, H756 or D759 of SEQ ID NO: 7, and an amino acid substitution at E615, H616 or N639 of SEQ ID NO: 7.

In some embodiments, the CRISPR nuclease comprises a sequence having at least 90% identity to the amino acid sequence set forth in SEQ ID NO: 8, and at least one RNA molecule comprises a sequence selected from the group consisting of SEQ ID NOs: 109-120.

In some embodiments, the CRISPR nuclease comprises a sequence having at least 90% identity to the amino acid sequence set forth in SEQ ID NO: 8 and at least one RNA molecule is a CRISPR RNA (crRNA) molecule comprising a guide sequence portion and a sequence selected from the group consisting of SEQ ID NOs: 109-112.

In some embodiments, the composition further comprises a transactivating CRISPR RNA (tracrRNA) molecule comprising a sequence set forth in the group consisting of SEQ ID NOs: 113-119.

In some embodiments, the CRISPR nuclease comprises a sequence having at least 90% identity to the amino acid sequence set forth in SEQ ID NO: 8 and at least one RNA molecule is a single-guide RNA (sgRNA) molecule comprising a guide sequence portion and a sequence selected from the group consisting of SEQ ID NOs: 109-120.

In some embodiments, the CRISPR nuclease comprises a sequence having at least 90% identity to the amino acid sequence of SEQ ID NO: 8, and wherein the CRISPR nuclease is a nickase formed by an amino acid substitution at D6, E524, H756 or D759 of SEQ ID NO: 8.

In some embodiments, the CRISPR nuclease comprises a sequence having at least 90% identity to the amino acid sequence of SEQ ID NO: 8, and wherein the CRISPR nuclease is a nickase formed by an amino acid substitution at D608, H609 or N632 of SEQ ID NO: 8.

In some embodiments, the CRISPR nuclease comprises a sequence having at least 90% identity to the amino acid sequence of SEQ ID NO: 8, and wherein the CRISPR nuclease is a catalytically inactive nuclease formed by an amino acid substitution at D6, E524, H756 or D759 of SEQ ID NO: 8, and an amino acid substitution at D608, H609 or N632 of SEQ ID NO: 8.

According to embodiments of the present invention, there is provided a non-naturally occurring composition comprising a CRISPR nuclease, wherein the CRISPR nuclease comprises an amino acid sequence corresponding to the amino acid sequence of at least one of Domain A, Domain B, Domain C, Domain D, Domain E, Domain F, Domain G, Domain H, or Doman I of OMNI-79 CRISPR nuclease (SEQ ID NO: 5).

In some embodiments, the CRISPR nuclease comprises a Domain A having at least 97% sequence identity to amino acids 1 to 40 of SEQ ID NO: 5.

In some embodiments, the CRISPR nuclease comprises a Domain B having at least 97% sequence identity to amino acids 41 to 76 of SEQ ID NO: 5.

In some embodiments, the CRISPR nuclease comprises a Domain C having at least 97% sequence identity to amino acids 77 to 228 of SEQ ID NO: 5.

In some embodiments, the CRISPR nuclease comprises a Domain D having at least 97% sequence identity to amino acids 229 to 446 of SEQ ID NO: 5.

In some embodiments, the CRISPR nuclease comprises a Domain E having at least 97% sequence identity to amino acids 447 to 507 of SEQ ID NO: 5.

In some embodiments, the CRISPR nuclease comprises a Domain F having at least 97% sequence identity to amino acids 539 to 648 of SEQ ID NO: 5.

In some embodiments, the CRISPR nuclease comprises a Domain G having at least 97% sequence identity to amino acids 665 to 822 of SEQ ID NO: 5.

In some embodiments, the CRISPR nuclease comprises a Domain H having at least 97% sequence identity to amino acids 823 to 921 of SEQ ID NO: 5.

In some embodiments, the CRISPR nuclease comprises a Domain I having at least 97% sequence identity to amino acids 922 to 1062 of SEQ ID NO: 5.

In some embodiments, the CRISPR nuclease sequence is at least 100-250, 250-500, 500-1000, or 1000-2000 amino acids in length.

A non-naturally occurring composition comprising a peptide, or a polynucleotide encoding the peptide, wherein the peptide comprises an amino acid sequence having at least 97% sequence identity to the amino acid sequence of at least one of Domain A, Domain B, Domain C, Domain D, Domain E, Domain F, Domain G, Domain H, or Doman I of OMNI-79 CRISPR nuclease.

According to embodiments of the present invention, there is provided a composition of an engineered, non-naturally occurring composition comprising a CRISPR associated system comprising:
one or more RNA molecules, or one or more nucleotide sequences encoding the one or more RNA molecules, the one or more RNA molecules comprising a guide sequence portion linked to a direct repeat sequence, wherein the guide sequence portion is capable of hybridizing with a target sequence; and
a CRISPR nuclease comprising an amino acid sequence having at least 90% identity to the amino acid sequence selected from the group consisting of SEQ ID NOs: 1-8 or a nucleic acid molecule comprising a sequence encoding the CRISPR nuclease; and
wherein the one or more RNA molecules hybridize to the target sequence, wherein the target sequence is adjacent to the 3′ end of a complimentary sequence of a Protospacer Adjacent Motif (PAM), and the one or more RNA molecules form a complex with the CRISPR nuclease.

According to embodiments of the present invention, there is provided a method of modifying a nucleotide sequence at a target site in a cell-free system or the genome of a cell comprising introducing into the cell any one of the compositions described herein.

In some embodiments, the cell is a eukaryotic cell or a prokaryotic cell.

In some embodiments, the eukaryotic cell is a mammalian cell.

In some embodiments, the mammalian cell is a human cell.

In some embodiments, the CRISPR nuclease forms a complex with the at least one RNA molecule or RNA molecules, and effects a DNA break in a DNA strand adjacent to a protospacer adjacent motif (PAM) sequence, and/or effects a DNA break in a DNA strand adjacent to a sequence that is complementary to the PAM sequence.

In some embodiments, the CRISPR nuclease comprises a sequence having at least 90% identity to SEQ ID NO: 1 and the PAM site is NGCNNT.

In some embodiments, the CRISPR nuclease is a nickase formed by an amino acid substitution at D24, E557, H785 or D788 of SEQ ID NO: 1, and effects a DNA break in a DNA strand adjacent to a PAM sequence.

In some embodiments, the CRISPR nuclease is a nickase formed by an amino acid substitution at E644, H645 or N668 of SEQ ID NO: 1, and effects a DNA break in a DNA strand adjacent to a sequence that is complementary to the PAM sequence.

In some embodiments, the CRISPR nuclease comprises a sequence having at least 90% identity to SEQ ID NO: 2 and the PAM sequence is NSHNAC.

In some embodiments, the CRISPR nuclease is a nickase formed by an amino acid substitution at D19, E528, H750 or D753 of SEQ ID NO: 2, and effects a DNA break in a DNA strand adjacent to a PAM sequence.

In some embodiments, the CRISPR nuclease is a nickase formed by an amino acid substitution at D609, H610 or N633 of SEQ ID NO: 2, and effects a DNA break in a DNA strand adjacent to a sequence that is complementary to the PAM sequence.

In some embodiments, the CRISPR nuclease comprises a sequence having at least 90% identity to SEQ ID NO: 3 and the PAM sequence is NRRCM.

In some embodiments, the CRISPR nuclease is a nickase formed by an amino acid substitution at D8, E503, H729 or D732 of SEQ ID NO: 3, and effects a DNA break in a DNA strand adjacent to a PAM sequence.

In some embodiments, the CRISPR nuclease is a nickase formed by an amino acid substitution at E584, H585 or N607 of SEQ ID NO: 3, and effects a DNA break in a DNA strand adjacent to a sequence that is complementary to the PAM sequence.

In some embodiments, the CRISPR nuclease comprises a sequence having at least 90% identity to SEQ ID NO. 4 and the PAM sequence is NGSNNT.

In some embodiments, the CRISPR nuclease is a nickase formed by an amino acid substitution at D12, E543, H770 or D773 of SEQ ID NO: 4, and effects a DNA break in a DNA strand adjacent to a PAM sequence.

In some embodiments, the CRISPR nuclease is a nickase formed by an amino acid substitution at E630, H631 or N654 of SEQ ID NO: 4, and effects a DNA break in a DNA strand adjacent to a sequence that is complementary to the PAM sequence.

In some embodiments, the CRISPR nuclease comprises a sequence having at least 90% identity to SEQ ID NO: 5 and the PAM sequence is NGR or NGG.

In some embodiments, the CRISPR nuclease is a nickase formed by an amino acid substitution at D8, E502, H735 or D738 of SEQ ID NO: 5, and effects a DNA break in a DNA strand adjacent to the PAM sequence.

In some embodiments, the CRISPR nuclease is a nickase formed by an amino acid substitution at D586, H587 or N610 of SEQ ID NO: 5, and effects a DNA break in a DNA strand adjacent to a sequence that is complementary to the PAM sequence.

In some embodiments, the CRISPR nuclease comprises a sequence having at least 90% identity to SEQ ID NO: 6 and the PAM sequence is NNRGAY.

In some embodiments, the CRISPR nuclease is a nickase formed by an amino acid substitution at D8, E523, H757 or D760 of SEQ ID NO: 6, and effects a DNA break in a DNA strand adjacent to the PAM sequence.

In some embodiments, the CRISPR nuclease is a nickase formed by an amino acid substitution at D607, H608 or N631 of SEQ ID NO: 6, and effects a DNA break in a DNA strand adjacent to a sequence that is complementary to the PAM sequence.

In some embodiments, the CRISPR nuclease comprises a sequence having at least 90% identity to SEQ ID NO: 7 and the PAM sequence is NRRAA.

In some embodiments, the CRISPR nuclease is a nickase formed by an amino acid substitution at D12, E527, H756 or D759 of SEQ ID NO: 7, and effects a DNA break in a DNA strand adjacent to the PAM sequence.

In some embodiments, the CRISPR nuclease is a nickase formed by an amino acid substitution at E615, H616 or N639 of SEQ ID NO: 7, and effects a DNA break in a DNA strand adjacent to a sequence that is complementary to the PAM sequence.

In some embodiments, the CRISPR nuclease comprises a sequence having at least 90% identity to SEQ ID NO: 8 and the PAM sequence is NRRNTT.

In some embodiments, the CRISPR nuclease is a nickase formed by an amino acid substitution at D6, E524, H756 or D759 of SEQ ID NO: 8, and effects a DNA break in a DNA strand adjacent to the PAM sequence.

In some embodiments, the CRISPR nuclease is a nickase formed by an amino acid substitution at D608, H609 or N632 of SEQ ID NO: 8, and effects a DNA break in a DNA strand adjacent to a sequence that is complementary to the PAM sequence.

According to some aspects of the invention, the disclosed compositions comprise a Clustered Regularly Interspaced Short Palindromic Repeats (CRISPR) nuclease and/or a nucleic acid molecule comprising a sequence encoding the same.

Tables 1a-1b list novel CRISPR nucleases, as well as substitutions at one or more positions within each nuclease which convert the nuclease to a nickase or catalytically dead nuclease.

Tables 2a-2d provide crRNA, tracrRNA, and single-guide RNA (sgRNA) sequences, and portions of crRNA, tracrRNA, and sgRNA sequences, that are compatible with each listed CRISPR nuclease. Accordingly, a crRNA molecule capable of binding and targeting an OMNI nuclease listed in Tables 2a-2d as part of a crRNA:tracrRNA complex may comprise any crRNA sequence listed in Tables 2a-2d. Similarly, a tracrRNA molecule capable of binding and targeting an OMNI nuclease listed in Tables 2a-2d as part of a crRNA:tracrRNA complex may comprise any tracrRNA sequence listed in Tables 2a-2d. Also, a single-guide RNA molecule capable of binding and targeting an OMNI nuclease listed in Tables 2a-2d may comprise any sequence listed in Tables 2a-2d.

For example, a crRNA molecule of OMNI-61 nuclease (SEQ ID NO: 2) may comprise a sequence of SEQ ID NOs: 30-32; a tracrRNA molecule of OMNI-61 nuclease may comprise a sequence of any one of SEQ ID NOs: 33-38; and a sgRNA molecule of OMNI-62 nuclease may comprise a sequence of any one of SEQ ID NOs: 30-39. Other crRNA molecules, tracrRNA molecules, or sgRNA molecules for each OMNI nuclease may be derived from the sequences listed in Tables 2a-2d in the same manner.

In some embodiments, the CRISPR nuclease comprises an amino acid sequence having at least 100%, 99%, 98%, 97%, 96%, 95%, 94%, 93%, 92%, 91%, 90%, 89%, 88%, 87%, 86%, 85%, 84%, 83%, or 82% amino acid sequence identity to a CRISPR nuclease as set forth in any of SEQ ID NOs: 1-8. In an embodiment the sequence encoding the CRISPR nuclease has at least 95% identity to a nucleic acid sequence selected from the group consisting of SEQ ID NOs: 9-24.

In some embodiments, the CRISPR nuclease comprises an amino acid sequence having at least 100%, 99%, 98%, 97%, 96%, 95%, 94%, 93%, 92%, 91%, 90%, 89%, 88%, 87%, 86%, 85%, 84%, 83%, 82%, 81%, 80%, 75% amino acid sequence identity to a CRISPR nucleases derived from Comamonadaceae bacterium NML00-0135, Demequina sediminicola, Fuerstia marisgermanicae, Nitrosomonas sp. Nm33, Novosphingobium sp. SYSU G00007, Paracoccus bengalensis, Parvibium lacunae, or Pelagicola sp. LXJ1103. Each possibility represents a separate embodiment.

In some embodiments, the CRISPR nuclease is a nickase having an inactivated RuvC domain created by an amino acid substitution at a position provided for the CRISPR nuclease in Tables 1a-1b.

In some embodiments, the CRISPR nuclease is a nickase having an inactivated HNH domain created by an amino acid substitution at a position provided for the CRISPR nuclease in Tables 1a-1b.

In some embodiments, the CRISPR nuclease is a catalytically dead nuclease having an inactivated RuvC domain and an inactivated HNH domain created by substitutions at the positions provided for the CRISPR nuclease in Tables 1a-1b.

For example, a nickase may be generated for the OMNI-59 nuclease by inactivating its RuvC domain by substituting an aspartic acid residue (D) in position 24 of the amino acid sequence of OMNI-59 (SEQ ID NO: 1) for another amino acid e.g. alanine (A). Substitution to any other amino acid is permissible for each of the amino acid positions indicated in Tables 1a-1b, except if the amino acid position is followed by an asterisk, which indicates that any substitution other than aspartic acid (D) to glutamic acid (E) or glutamic acid (E) to aspartic acid (D) results in inactivation. For example, a nickase may be generated for the OMNI-79 nuclease (SEQ ID NO: 5) by inactivating its HNH domain by substituting an aspartic acid residue (D) in position 586 of the amino acid sequence of OMNI-79 for an amino acid other than glutamic acid (E), e.g. for alanine (A). Other nickases or catalytically dead nucleases can be generated using the same notation in Tables 1a-1b.

In some embodiments, the CRISPR nuclease utilizes a protospacer adjacent motif (PAM) sequence provided for the CRISPR nuclease in Tables 3a-3c.

According to some aspects of the invention, the disclosed methods provide a method of modifying a nucleotide sequence at a DNA target site in a cell-free system or the genome of a cell comprising introducing into the cell any one of the CRISPR nucleases and a suitable crRNA:tracrRNA complex or sgRNA molecule.

In some embodiments, the CRISPR nuclease effects a DNA break in a DNA strand adjacent to a protospacer adjacent motif (PAM) sequence provided for the CRISPR nuclease in Tables 3a-3c, and effects a DNA break in a DNA strand adjacent to a sequence that is complementary to the PAM sequence. For example, the OMNI-79 nuclease with the appropriate targeting sgRNA molecule or crRNA:tracrRNA complex is capable of forming a DNA break in a strand adjacent to a NGR or NGG sequence and in a DNA strand adjacent to a sequence that is complementary to a NGR or NGG sequence. In some embodiments, the DNA strand is within a nucleus of a cell.

According to some aspects of the invention, the disclosed compositions comprise DNA constructs or a vector system comprising nucleotide sequences that encode the CRISPR nuclease or variant CRISPR nuclease. In some embodiments, the nucleotide sequence that encodes the CRISPR nuclease or variant CRISPR nuclease is operably linked to a promoter that is operable in the cells of interest. In some embodiments, the cell of interest is a eukaryotic cell. In some embodiments the cell of interest is a mammalian cell. In some embodiments, the nucleic acid sequence encoding the engineered CRISPR nuclease is codon optimized for use in cells from a particular organism. In some embodiments, the nucleic acid sequence encoding the nuclease is codon optimized for E. coli. In some embodiments, the nucleic acid sequence encoding the nuclease is codon optimized for eukaryotic cells. In some embodiments, the nucleic acid sequence encoding the nuclease is codon optimized for mammalian cells.

In some embodiments, the composition comprises a recombinant nucleic acid, comprising a heterologous promoter operably linked to a polynucleotide encoding a CRISPR enzyme having at least 100%, 99%, 98%, 97%, 96%, 95%, 94%, 93%, 92%, 91%, 90% identity to any of SEQ ID NOs: 1-8. Each possibility represents a separate embodiment.

In an embodiment of the composition, the CRISPR nuclease has at least 75%, 80%, 85%, 90%, 95%, or 97% identity to the amino acid sequence as set forth in SEQ ID NO: 1 or the sequence encoding the CRISPR nuclease has at least a 75%, 80%, 85%, 90%, 95%, or 97% sequence identity to a nucleotide sequence selected from the group consisting of SEQ ID NOs: 9 and 17.

In an embodiment of the composition, the CRISPR nuclease has at least 75%, 80%, 85%, 90%, 95%, or 97% identity to the amino acid sequence as set forth in SEQ ID NO: 2 or the sequence encoding the CRISPR nuclease has at least a 75%, 80%, 85%, 90%, 95%, or 97% sequence identity to a nucleotide sequence selected from the group consisting of SEQ ID NOs: 10 and 18.

In an embodiment of the composition, the CRISPR nuclease has at least 75%, 80%, 85%, 90%, 95%, or 97% identity to the amino acid sequence as set forth in SEQ ID NO: 3 or the sequence encoding the CRISPR nuclease has at least a 75%, 80%, 85%, 90%, 95%, or 97% sequence identity to a nucleotide sequence selected from the group consisting of SEQ ID NOs: 11 and 19.

In an embodiment of the composition, the CRISPR nuclease has at least 75%, 80%, 85%, 90%, 95%, or 97% identity to the amino acid sequence as set forth in SEQ ID NO: 4 or the sequence encoding the CRISPR nuclease has at least a 75%, 80%, 85%, 90%, 95%, or 97% sequence identity to a nucleotide sequence selected from the group consisting of SEQ ID NOs: 12 and 20.

In an embodiment of the composition, the CRISPR nuclease has at least 75%, 80%, 85%, 90%, 95%, or 97% identity to the amino acid sequence as set forth in SEQ ID NO: 5 or the sequence encoding the CRISPR nuclease has at least a 75%, 80%, 85%, 90%, 95%, or 97% sequence identity to a nucleotide sequence selected from the group consisting of SEQ ID NOs: 13 and 21.

In an embodiment of the composition, the CRISPR nuclease has at least 75%, 80%, 85%, 90%, 95%, or 97% identity to the amino acid sequence as set forth in SEQ ID NO: 6 or the sequence encoding the CRISPR nuclease has at least a 75%, 80%, 85%, 90%, 95%, or 97% sequence identity to a nucleotide sequence selected from the group consisting of SEQ ID NOs: 14 and 22.

In an embodiment of the composition, the CRISPR nuclease has at least 75%, 80%, 85%, 90%, 95%, or 97% identity to the amino acid sequence as set forth in SEQ ID NO: 7 or the sequence encoding the CRISPR nuclease has at least a 75%, 80%, 85%, 90%, 95%, or 97% sequence identity to a nucleotide sequence selected from the group consisting of SEQ ID NOs: 15 and 23.

In an embodiment of the composition, the CRISPR nuclease has at least 75%, 80%, 85%, 90%, 95%, or 97% identity to the amino acid sequence as set forth in SEQ ID NO: 8 or the sequence encoding the CRISPR nuclease has at least a 75%, 80%, 85, 90%, 95%, or 97% sequence identity to a nucleotide sequence selected from the group consisting of SEQ ID NOs: 16 and 24.

According to some embodiments, there is provided an engineered or non-naturally occurring composition comprising a CRISPR nuclease comprising a sequence having at least 100%, 99%, 98%, 97%, 96%, 95%, 94%, 93%, 92%, 91%, 90%, 85%, 80% identity to the amino acid sequence selected from the group consisting of SEQ ID NOs: 1-8 or a nucleic acid molecule comprising a sequence encoding the CRISPR nuclease. Each possibility represents a separate embodiment.

In an embodiment, the CRISPR nuclease is engineered or non-naturally occurring. The CRISPR nuclease may also be recombinant. Such CRISPR nucleases are produced using laboratory methods (molecular cloning) to bring together genetic material from multiple sources, creating sequences that would not otherwise be found in biological organisms.

In an embodiment, the CRISPR nuclease of the invention exhibits increased specificity to a target site compared to a SpCas9 nuclease when complexed with the one or more RNA molecules.

In an embodiment, the complex of the CRISPR nuclease of the invention and one or more RNA molecules exhibits at least maintained on-target editing activity of the target site and reduced off-target activity compared to SpCas9 nuclease.

In an embodiment, the CRISPR nuclease further comprises an RNA-binding portion capable of interacting with a DNA-targeting RNA molecule (gRNA) and an activity portion that exhibits site-directed enzymatic activity.

In an embodiment, the composition further comprises a DNA-targeting RNA molecule or a DNA polynucleotide encoding a DNA-targeting RNA molecule, wherein the DNA-targeting RNA molecule comprises a nucleotide sequence that is complementary to a sequence in a target region, wherein the DNA-targeting RNA molecule and the CRISPR nuclease do not naturally occur together.

In an embodiment, the DNA-targeting RNA molecule further comprises a nucleotide sequence that can form a complex with a CRISPR nuclease.

This invention also provides a non-naturally occurring composition comprising a CRISPR associated system comprising:
a) one or more RNA molecules comprising a guide sequence portion linked to a direct repeat sequence, wherein the guide sequence is capable of hybridizing with a target sequence, or one or more nucleotide sequences encoding the one or more RNA molecules; and
b) a CRISPR nuclease comprising an amino acid sequence having at least 95% identity to the amino acid sequence selected from the group consisting of SEQ ID NOs: 1-8 or a nucleic acid molecule comprising a sequence encoding the CRISPR nuclease; wherein the one or more RNA molecules hybridize to the target sequence, wherein the target sequence is adjacent to the 3′ end of a complimentary sequence of a Protospacer Adjacent Motif (PAM), and the one or more RNA molecules form a complex with the RNA-guided nuclease.

In an embodiment, the composition further comprises an RNA molecule comprising a nucleotide sequence that can form a complex with a CRISPR nuclease (tracrRNA) or a DNA polynucleotide comprising a sequence encoding an RNA molecule that can form a complex with the CRISPR nuclease.

In an embodiment, the composition further comprises a donor template for homology directed repair (HDR).

In an embodiment, the composition is capable of editing the target region in the genome of a cell.

In an embodiment of the composition:
a) the CRISPR nuclease has at least 100%, 99%, 98%, 97%, 96%, 95%, 94%, 93%, 92%, 91%, 900, 85%, 80% identity to SEQ ID NO: 1, and the RNA molecule comprises a sequence selected from SEQ ID NOs: 25-29;
b) the CRISPR nuclease has at least 100%, 99%, 98%, 97%, 96%, 95%, 94%, 93%, 92%, 91%, 90%, 85%, 80% identity to SEQ ID NO: 2, and the RNA molecule comprises a sequence selected from SEQ ID NOs: 30-39;
c) the CRISPR nuclease has at least 100%, 99%, 98%, 97%, 96%, 95%, 94%, 93%, 92%, 91%, 90%, 85%, 80% identity to SEQ ID NO: 3, and the RNA molecule comprises a sequence selected from SEQ ID NOs: 40-52;
d) the CRISPR nuclease has at least 100%, 99%, 98%, 97%, 96%, 95%, 94%, 93%, 92%, 91%, 900, 85%, 80% identity to SEQ ID NO: 4, and the RNA molecule comprises a sequence selected from SEQ ID NOs: 53-62;
e) the CRISPR nuclease has at least 100%, 99%, 98%, 97%, 96%, 95%, 94%, 93%, 92%, 91%, 90%, 85%, 80% identity to SEQ ID NO: 5, and the RNA molecule comprises a sequence selected from SEQ ID NOs: 63-90;
f) the CRISPR nuclease has at least 100%, 99%, 98%, 97%, 96%, 95%, 94%, 93%, 92%, 91%, 90%, 85%, 80% identity to SEQ ID NO: 6, and the RNA molecule comprises a sequence selected from SEQ ID NOs: 91-98;
g) the CRISPR nuclease has at least 100%, 99%, 98%, 97%, 96%, 95%, 94%, 93%, 92%, 91%, 90%, 85%, 80% identity to SEQ ID NO: 7, and the RNA molecule comprises a sequence selected from SEQ ID NOs: 99-108; or
h) the CRISPR nuclease has at least 100%, 99%, 98%, 97%, 96%, 95%, 94%, 93%, 92%, 91%, 90%, 85%, 80% identity to SEQ ID NO: 8, and the RNA molecule comprises a sequence selected from SEQ ID NOs: 109-120.

According to some embodiments, there is provided a non-naturally occurring composition comprising:
(a) a CRISPR nuclease, or a polynucleotide encoding the CRISPR nuclease, comprising: an RNA-binding portion; and
   an activity portion that exhibits site-directed enzymatic activity, wherein the CRISPR nuclease has at least 100%, 99%, 98%, 97%, 96%, 95%, 94%, 93%, 92%, 91%, 90%, 85%, 80% identity to any of SEQ ID NOs: 1-8; and
(b) one or more RNA molecules or a DNA polynucleotide encoding the one or more RNA molecules comprising:
   i) a DNA-targeting RNA sequence, comprising a nucleotide sequence that is complementary to a sequence in a target DNA sequence; and
   ii) a protein-binding RNA sequence, capable of interacting with the RNA-binding portion of the CRISPR nuclease,
wherein the DNA targeting RNA sequence and the CRISPR nuclease do not naturally occur together. Each possibility represents a separate embodiment.

In some embodiments, there is provided a single RNA molecule comprising the DNA-targeting RNA sequence and the protein-binding RNA sequence, wherein the RNA molecule can form a complex with the CRISPR nuclease and serve as the DNA targeting module. In some embodiments, the RNA molecule has a length of up to 1000 bases, 900 bases, 800 bases, 700 bases, 600 bases, 500 bases, 400 bases, 300 bases, 200 bases, 100 bases, 50 bases. Each possibility represents a separate embodiment. In some embodiments, a first RNA molecule comprising the DNA-targeting RNA sequence and a second RNA molecule comprising the protein-binding RNA sequence interact by base pairing or alternatively fused together to form one or more RNA molecules that complex with the CRISPR nuclease and serve as the DNA targeting module.

In some embodiments, the CRISPR nuclease has at least 100%, 99%, 98%, 97%, 96%, 95%, 94%, 93%, 92%, 91%, 90%, 85%, 80% identity to SEQ ID NO: 1, and the RNA molecule comprises a sequence selected from SEQ ID NOs: 25-29.

In some embodiments, the CRISPR nuclease has at least 100%, 99%, 98%, 97%, 96%, 95%, 94%, 93%, 92%, 91%, 90%, 85%, 80% identity to SEQ ID NO: 2, and the RNA molecule comprises a sequence selected from SEQ ID NOs: 30-39.

In some embodiments, the CRISPR nuclease has at least 100%, 99%, 98%, 97%, 96%, 95%, 94%, 93%, 92%, 91%, 90%, 85%, 80% identity to SEQ ID NO: 3, and the RNA molecule comprises a sequence selected from SEQ ID NOs: 40-52.

In some embodiments, the CRISPR nuclease has at least 100%, 99%, 98%, 97%, 96%, 95%, 94%, 93%, 92%, 91%, 90%, 85%, 80% identity to SEQ ID NO: 4, and the RNA molecule comprises a sequence selected from SEQ ID NOs: 53-62.

In some embodiments, the CRISPR nuclease has at least 100%, 99%, 98%, 97%, 96%, 95%, 94%, 93%, 92%, 91%, 90%, 85%, 80% identity to SEQ ID NO: 5, and the RNA molecule comprises a sequence selected from SEQ ID NOs: 63-90.

In some embodiments, the CRISPR nuclease has at least 100%, 99%, 98%, 97%, 96%, 95%, 94%, 93%, 92%, 91%, 90%, 85%, 80% identity to SEQ ID NO: 6, and the RNA molecule comprises a sequence selected from SEQ ID NOs: 91-98.

In some embodiments, the CRISPR nuclease has at least 100%, 99%, 98%, 97%, 96%, 95%, 94%, 93%, 92%, 91%, 90%, 85%, 80% identity to SEQ ID NO: 7, and the RNA molecule comprises a sequence selected from SEQ ID NOs: 99-108.

In some embodiments, the CRISPR nuclease has at least 100%, 99%, 98%, 97%, 96%, 95%, 94%, 93%, 92%, 91%, 90%, 85%, 80% identity to SEQ ID NO: 8, and the RNA molecule comprises a sequence selected from SEQ ID NOs: 109-120.

This invention also provides a non-naturally occurring composition comprising:
a) a CRISPR nuclease comprising a sequence having at least 95% identity to the amino acid sequence selected from the group consisting of SEQ ID NOs: 1-8 or a nucleic acid molecule comprising a sequence encoding the CRISPR nuclease; and
b) one or more RNA molecules, or one or more DNA polynucleotide encoding the one or more RNA molecules, comprising at least one of:
   i) a nuclease-binding RNA nucleotide sequence capable of interacting with/binding to the CRISPR nuclease; and
   ii) a DNA-targeting RNA nucleotide sequence comprising a sequence complementary to a sequence in a target DNA sequence,
wherein the CRISPR nuclease is capable of complexing with the one or more RNA molecules to form a complex capable of hybridizing with the target DNA sequence.

In an embodiment, the CRISPR nuclease and the one or more RNA molecules form a CRISPR complex that is capable of binding to the target DNA sequence to effect cleavage of the target DNA sequence.

In an embodiment, the CRISPR nuclease and at least one of the one or more RNA molecules do not naturally occur together.

In an embodiment:
a) the CRISPR nuclease comprises an RNA-binding portion and an activity portion that exhibits site-directed enzymatic activity;
b) the DNA-targeting RNA nucleotide sequence comprises a nucleotide sequence that is complementary to a sequence in a target DNA sequence; and
c) the nuclease-binding RNA nucleotide sequence comprises a sequence that interacts with the RNA-binding portion of the CRISPR nuclease.

In an embodiment, the nuclease-binding RNA nucleotide sequence and the DNA-targeting RNA nucleotide sequence are on a single guide RNA molecule (sgRNA), wherein the sgRNA molecule can form a complex with the CRISPR nuclease and serve as the DNA targeting module.

In an embodiment, the nuclease-binding RNA nucleotide sequence is on a first RNA molecule and the DNA-targeting RNA nucleotide sequence is on a single guide RNA molecule, and wherein the first and second RNA sequence interact by base-pairing or are fused together to form one or more RNA molecules or sgRNA that complex with the CRISPR nuclease and serve as the targeting module.

In an embodiment, the sgRNA has a length of up to 1000 bases, 900 bases, 800 bases, 700 bases, 600 bases, 500 bases, 400 bases, 300 bases, 200 bases, 100 bases, 50 bases.

In an embodiment, the composition further comprises a donor template for homology directed repair (HDR).

In some embodiments, (a) the CRISPR nuclease has at least 100%, 99%, 98%, 97%, 96%, 95%, 94%, 93%, 92%, 91%, 90%, 85%, 80% identity to SEQ ID NO: 1, or (b) the nucleic acid molecule comprising a sequence encoding the CRISPR nuclease comprises a sequence of at least a 95% sequence identity to the nucleic acid sequence as set forth in SEQ ID NO: 9 or 17 and the PAM is NGCNNT. In some embodiments, the DNA-targeting RNA molecule comprises a sequence selected from SEQ ID NOs: 25-29.

In some embodiments, (a) the CRISPR nuclease has at least 100%, 99%, 98%, 97%, 96%, 95%, 94%, 93%, 92%, 91%, 90%, 85%, 80% identity to SEQ ID NO: 2, or (b) the nucleic acid molecule comprising a sequence encoding the CRISPR nuclease comprises a sequence of at least a 95% sequence identity to the nucleic acid sequence as set forth in SEQ ID NO: 10 or 18 and the PAM is NSHNAC. In some embodiments, the DNA-targeting RNA molecule comprises a sequence selected from SEQ ID NOs: 30-39.

In some embodiments, (a) the CRISPR nuclease has at least 100%, 99%, 98%, 97%, 96%, 95%, 94%, 93%, 92%, 91%, 90%, 85%, 80% identity to SEQ ID NO: 3, or (b) the nucleic acid molecule comprising a sequence encoding the CRISPR nuclease comprises a sequence of at least a 95% sequence identity to the nucleic acid sequence as set forth in SEQ ID NO: 11 or 19 and the PAM is NRRCM. In some embodiments, the DNA-targeting RNA molecule comprises a sequence selected from SEQ ID NOs: 40-52.

In some embodiments, (a) the CRISPR nuclease has at least 100%, 99%, 98%, 97%, 96%, 95%, 94%, 93%, 92%, 91%, 90%, 85%, 80% identity to SEQ ID NO: 4, or (b) the nucleic acid molecule comprising a sequence encoding the CRISPR nuclease comprises a sequence of at least a 95% sequence identity to the nucleic acid sequence as set forth in SEQ ID NO: 12 or 20 and the PAM is NGSNNT. In some embodiments, the DNA-targeting RNA molecule comprises a sequence selected from SEQ ID NOs: 53-62.

In some embodiments, (a) the CRISPR nuclease has at least 100%, 99%, 98%, 97%, 96%, 95%, 94%, 93%, 92%, 91%, 90%, 85%, 80% identity to SEQ ID NO: 5, or (b) the nucleic acid molecule comprising a sequence encoding the CRISPR nuclease comprises a sequence of at least a 95% sequence identity to the nucleic acid sequence as set forth in SEQ ID NO: 13 or 21 and the PAM is NGR or NGG. In some embodiments, the DNA-targeting RNA molecule comprises a sequence selected from SEQ ID NOs: 63-90.

In some embodiments, (a) the CRISPR nuclease has at least 100%, 99%, 98%, 97%, 96%, 95%, 94%, 93%, 92%, 91%, 90%, 85%, 80% identity to SEQ ID NO: 6, or (b) the nucleic acid molecule comprising a sequence encoding the CRISPR nuclease comprises a sequence of at least a 95% sequence identity to the nucleic acid sequence as set forth in SEQ ID NO: 14 or 22 and the PAM is NNRGAY. In some embodiments, the DNA-targeting RNA molecule comprises a sequence selected from SEQ ID NOs: 91-98.

In some embodiments, (a) the CRISPR nuclease has at least 100%, 99%, 98%, 97%, 96%, 95%, 94%, 93%, 92%, 91%, 90%, 85%, 80% identity to SEQ ID NO: 7, or (b) the nucleic acid molecule comprising a sequence encoding the CRISPR nuclease comprises a sequence of at least a 95% sequence identity to the nucleic acid sequence as set forth in SEQ ID NO: 15 or 23 and the PAM is NRRAA. In some embodiments, the DNA-targeting RNA molecule comprises a sequence selected from SEQ ID NOs: 99-108.

In some embodiments, (a) the CRISPR nuclease has at least 100%, 99%, 98%, 97%, 96%, 95%, 94%, 93%, 92%, 91%, 90%, 85%, 80% identity to SEQ ID NO: 8, or (b) the nucleic acid molecule comprising a sequence encoding the CRISPR nuclease comprises a sequence of at least a 95% sequence identity to the nucleic acid sequence as set forth in SEQ ID NO: 16 or 24 and the PAM is NRRNTT. In some embodiments, the DNA-targeting RNA molecule comprises a sequence selected from SEQ ID NOs: 109-120.

In an embodiment, the CRISPR nuclease comprises 1-10, 10-20, 20-30, 30-40, 40-50, 50-60, 60-70, 70-80, 80-90, 90-100, 100-110, 110-120, 120-130, 130-140, or 140-150 amino acid substitutions, deletions, and/or insertions compared to the amino acid sequence of the wild-type of the CRISPR nuclease.

In an embodiment, the CRISPR nuclease exhibits at least 2%, 5%, 7% 10%, 15%, 20%, 25%, 30%, or 35% increased specificity compared the wild-type of the CRISPR nuclease.

In an embodiment, the CRISPR nuclease exhibits at least 2%, 5%, 7% 10%, 15%, 20%, 25%, 30%, or 35% increased activity compared the wild-type of the CRISPR nuclease.

In an embodiment, the CRISPR nuclease has altered PAM specificity compared to the wild-type of the CRISPR nuclease.

In an embodiment, the CRISPR nuclease is non-naturally occurring.

In an embodiment, the CRISPR nuclease is engineered and comprises unnatural or synthetic amino acids.

In an embodiment, the CRISPR nuclease is engineered and comprises one or more of a nuclear localization sequences (NLS), cell penetrating peptide sequences, and/or affinity tags.

In an embodiment, the CRISPR nuclease comprises one or more nuclear localization sequences of sufficient strength to drive accumulation of a CRISPR complex comprising the CRISPR nuclease in a detectable amount in the nucleus of a eukaryotic cell.

This invention also provides a method of modifying a nucleotide sequence at a target site in a cell-free system or the genome of a cell comprising introducing into the cell any of the compositions of the invention.

In an embodiment, the cell is a eukaryotic cell.

In another embodiment, the cell is a prokaryotic cell.

In some embodiments, the one or more RNA molecules further comprises an RNA sequence comprising a nucleotide molecule that can form a complex with the RNA nuclease (tracrRNA) or a DNA polynucleotide encoding an RNA molecule comprising a nucleotide sequence that can form a complex with the CRISPR nuclease.

In an embodiment, the CRISPR nuclease comprises 1, 2, 3, 4, 5, 6, 7, 8, 9, 10, or more NLSs at or near the amino-terminus, 1, 2, 3, 4, 5, 6, 7, 8, 9, 10, or more NLSs at or near carboxy-terminus, or a combination of 1, 2, 3, 4, 5, 6, 7, 8, 9, 10, or more NLSs at or near the amino-terminus and 1, 2, 3, 4, 5, 6, 7, 8, 9, 10, or more NLSs at or near carboxy-terminus. In an embodiment 1-4 NLSs are fused with the CRISPR nuclease. In an embodiment, an NLS is located within the open-reading frame (ORF) of the CRISPR nuclease.

Methods of fusing an NLS at or near the amino-terminus, at or near carboxy-terminus, or within the ORF of an expressed protein are well known in the art. As an example, to fuse an NLS to the amino-terminus of a CRISPR nuclease, the nucleic acid sequence of the NLS is placed immediately after the start codon of the CRISPR nuclease on the nucleic acid encoding the NLS-fused CRISPR nuclease. Conversely, to fuse an NLS to the carboxy-terminus of a CRISPR nuclease the nucleic acid sequence of the NLS is placed after the codon encoding the last amino acid of the CRISPR nuclease and before the stop codon.

Any combination of NLSs, cell penetrating peptide sequences, and/or affinity tags at any position along the ORF of the CRISPR nuclease is contemplated in this invention.

The amino acid sequences and nucleic acid sequences of the CRISPR nucleases provided herein may include NLS and/or TAGs inserted so as to interrupt the contiguous amino acid or nucleic acid sequences of the CRISPR nucleases.

In an embodiment, the one or more NLSs are in tandem repeats.

In an embodiment, the one or more NLSs are considered in proximity to the N- or C-terminus when the nearest amino acid of the NLS is within about 1, 2, 3, 4, 5, 10, 15, 20, 25, 30, 40, 50, or more amino acids along the polypeptide chain from the N- or C-terminus.

As discussed, the CRISPR nuclease may be engineered to comprise one or more of a nuclear localization sequences (NLS), cell penetrating peptide sequences, and/or affinity tags.

In an embodiment, the CRISPR nuclease exhibits increased specificity to a target site compared to the wild-type of the CRISPR nuclease when complexed with the one or more RNA molecules.

In an embodiment, the complex of the CRISPR nuclease and one or more RNA molecules exhibits at least maintained on-target editing activity of the target site and reduced off-target activity compared to the wild-type of the CRISPR nuclease.

In an embodiment, the composition further comprises a recombinant nucleic acid molecule comprising a heterologous promoter operably linked to the nucleotide acid molecule comprising the sequence encoding the CRISPR nuclease.

In an embodiment, the CRISPR nuclease or nucleic acid molecule comprising a sequence encoding the CRISPR nuclease is non-naturally occurring or engineered.

This invention also provides a non-naturally occurring or engineered composition comprising a vector system comprising the nucleic acid molecule comprising a sequence encoding any of the CRISPR nucleases of the invention.

This invention also provides use of any of the compositions of the invention for the treatment of a subject afflicted with a disease associated with a genomic mutation comprising modifying a nucleotide sequence at a target site in the genome of the subject.

This invention provides a method of modifying a nucleotide sequence at a target site in the genome of a mammalian cell comprising introducing into the cell (i) a composition comprising a CRISPR nuclease having at least 95% identity to an amino acid sequence selected from the group consisting of SEQ ID NOs: 1-8 or a nucleic acid molecule comprising a sequence encoding a CRISPR nuclease which sequence has at least 95% identity to a nucleic acid sequence selected from the group consisting of SEQ ID NOs: 9-24 and (ii) a DNA-targeting RNA molecule, or a DNA polynucleotide encoding a DNA-targeting RNA molecule, comprising a nucleotide sequence that is complementary to a sequence in the target DNA.

In some embodiments, the method is performed ex vivo. In some embodiments, the method is performed in vivo. In some embodiments, some steps of the method are performed ex vivo and some steps are performed in vivo. In some embodiments the mammalian cell is a human cell.

In an embodiment, the method further comprises introducing into the cell: (iii) an RNA molecule comprising a nuclease-binding RNA sequence or a DNA polynucleotide encoding an RNA molecule comprising a nuclease-binding RNA that interacts with the CRISPR nuclease.

In an embodiment, the DNA targeting RNA molecule is a crRNA molecule suitable to form an active complex with the CRISPR nuclease.

In an embodiment, the RNA molecule comprising a nuclease-binding RNA sequence is a tracrRNA molecule suitable to form an active complex with the CRISPR nuclease.

In an embodiment, the DNA-targeting RNA molecule and the RNA molecule comprising a nuclease-biding RNA sequence are fused in the form of a single guide RNA molecule.

In an embodiment, the method further comprises introducing into the cell: (iv) an RNA molecule comprising a sequence complementary to a protospacer sequence.

In an embodiment, the CRISPR nuclease forms a complex with the one or more RNA molecules and effects a double strand break in the 3′ of a Protospacer Adjacent Motif (PAM).

In an embodiment, the CRISPR nuclease forms a complex with the one or more RNA molecules and effects a double strand break in the 5′ of a Protospacer Adjacent Motif (PAM).

In some embodiments, (a) the CRISPR nuclease has at least 100%, 99%, 98%, 97%, 96%, 95%, 94%, 93%, 92%, 91%, 90%, 85%, 80% identity to SEQ ID NO: 1, or (b) the nucleic acid molecule comprising a sequence encoding the CRISPR nuclease comprises a sequence of at least a 95% sequence identity to the nucleic acid sequence as set forth in SEQ ID NO: 9 or 17 and the PAM is NGCNNT. In some embodiments, the DNA-targeting RNA molecule comprises a sequence selected from SEQ ID NOs: 25-29.

In some embodiments, (a) the CRISPR nuclease has at least 100%, 990, 98%, 97%, 96%, 95%, 94%, 93%, 92%, 91%, 90%, 85%, 80% identity to SEQ ID NO: 2, or (b) the nucleic acid molecule comprising a sequence encoding the CRISPR nuclease comprises a sequence of at least a 95% sequence identity to the nucleic acid sequence as set forth in SEQ ID NO: 10 or 18 and the PAM is NSHNAC. In some embodiments, the DNA-targeting RNA molecule comprises a sequence selected from SEQ ID NOs: 30-39.

In some embodiments, (a) the CRISPR nuclease has at least 100%, 99%, 98%, 97%, 96%, 95%, 94%, 93%, 92%, 91%, 90%, 85%, 80% identity to SEQ ID NO: 3, or (b) the nucleic acid molecule comprising a sequence encoding the CRISPR nuclease comprises a sequence of at least a 95% sequence identity to the nucleic acid sequence as set forth in SEQ ID NO: 11 or 19 and the PAM is NRRCM. In some embodiments, the DNA-targeting RNA molecule comprises a sequence selected from SEQ ID NOs: 40-52.

In some embodiments, (a) the CRISPR nuclease has at least 100%, 99%, 98%, 97%, 96%, 95%, 94%, 93%, 92%, 91%, 90%, 85%, 80% identity to SEQ ID NO: 4, or (b) the nucleic acid molecule comprising a sequence encoding the CRISPR nuclease comprises a sequence of at least a 95% sequence identity to the nucleic acid sequence as set forth in SEQ ID NO: 12 or 20 and the PAM is NGSNNT. In some embodiments, the DNA-targeting RNA molecule comprises a sequence selected from SEQ ID NOs: 53-62.

In some embodiments, (a) the CRISPR nuclease has at least 100%, 99%, 98%, 97%, 96%, 95%, 94%, 93%, 92%, 91%, 90%, 85%, 80% identity to SEQ ID NO: 5, or (b) the nucleic acid molecule comprising a sequence encoding the CRISPR nuclease comprises a sequence of at least a 95% sequence identity to the nucleic acid sequence as set forth in SEQ ID NO: 13 or 21 and the PAM is NGR or NGG. In some embodiments, the DNA-targeting RNA molecule comprises a sequence selected from SEQ ID NOs: 63-90.

In some embodiments, (a) the CRISPR nuclease has at least 100%, 99%, 98%, 97%, 96%, 95%, 94%, 93%, 92%, 91%, 90%, 85%, 80% identity to SEQ ID NO: 6, or (b) the nucleic acid molecule comprising a sequence encoding the CRISPR nuclease comprises a sequence of at least a 95% sequence identity to the nucleic acid sequence as set forth in SEQ ID NO: 14 or 22 and the PAM is NNRGAY. In some embodiments, the DNA-targeting RNA molecule comprises a sequence selected from SEQ ID NOs: 91-98.

In some embodiments, (a) the CRISPR nuclease has at least 100%, 99%, 98%, 97%, 96%, 95%, 94%, 93%, 92%, 91%, 90%, 85%, 80% identity to SEQ ID NO: 7, or (b) the nucleic acid molecule comprising a sequence encoding the CRISPR nuclease comprises a sequence of at least a 95% sequence identity to the nucleic acid sequence as set forth in SEQ ID NO: 15 or 23 and the PAM is NRRAA. In this some embodiments, the DNA-targeting RNA molecule comprises a sequence selected from SEQ ID NOs: 99-108.

In some embodiments, (a) the CRISPR nuclease has at least 100%, 99%, 98%, 97%, 96%, 95%, 94%, 93%, 92%, 91%, 90%, 85%, 80% identity to SEQ ID NO: 8, or (b) the nucleic acid molecule comprising a sequence encoding the CRISPR nuclease comprises a sequence of at least a 95% sequence identity to the nucleic acid sequence as set forth in SEQ ID NO: 16 or 24 and the PAM is NRRNTT. In some embodiments, the DNA-targeting RNA molecule comprises a sequence selected from SEQ ID NOs: 109-120.

In an embodiment of any of the methods described herein, the method is for treating a subject afflicted with a disease associated with a genomic mutation comprising modifying a nucleotide sequence at a target site in the genome of the subject.

In an embodiment, the method comprises first selecting a subject afflicted with a disease associated with a genomic mutation and obtaining the cell from the subject.

This invention also provides a modified cell or cells obtained by any of the methods described herein. In an embodiment these modified cell or cells are capable of giving rise to progeny cells. In an embodiment these modified cell or cells are capable of giving rise to progeny cells after engraftment.

This invention also provides a composition comprising these modified cells and a pharmaceutically acceptable carrier. Also provided is an in vitro or ex vivo method of preparing this, comprising mixing the cells with the pharmaceutically acceptable carrier.

### DNA-Targeting RNA Molecules

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence. Accordingly, a molecule comprising a “guide sequence portion” is a type of targeting molecule. Throughout this application, the terms “guide molecule,” “RNA guide molecule,” “guide RNA molecule,” and “gRNA molecule” are synonymous with a molecule comprising a guide sequence portion, and the term “spacer” is synonymous with a “guide sequence portion.”

In embodiments of the present invention, the CRISPR nuclease has its greatest cleavage activity when used with an RNA molecule comprising a guide sequence portion having 20, 21, 22, 23, 24, 25, 26, 27, 28, 29, or 30 nucleotides.

In some embodiments, the OMNI-79 CRISPR nuclease has its greatest cleavage activity when used with an RNA molecule comprising a guide sequence portion having 25-26 nucleotides, compared to its cleavage activity when used with an RNA molecule comprising a guide sequence portion having 24 or fewer nucleotides, and/or 27 or more nucleotides.

According to some aspects of the invention, the disclosed methods comprise a method of modifying a nucleotide sequence at a target site in a cell-free system or the genome of a cell comprising introducing into the cell the composition of any one of the embodiments described herein.

In some embodiments, the cell is a eukaryotic cell, preferably a mammalian cell or a plant cell.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, cell dysregulation and oncology diseases and disorders, inflammation and immune-related diseases and disorders, metabolic, liver, kidney and protein diseases and disorders, muscular and skeletal diseases and disorders, dermatological diseases and disorders, neurological and neuronal diseases and disorders, and ocular diseases and disorders.

In some embodiments, the mutation disorder is beta thalassemia or sickle cell anemia.

In some embodiments, the allele associated with the disease is BCL11A.

### OMNI-79 CRISPR Nuclease Domains

The characteristic targeted nuclease activity of a CRISPR nuclease is imparted by the various functions of its specific domains. In this application the OMNI-79 domains are defined as Domain A, Domain B, Domain C, Domain D, Domain E, Domain F, Domain G, Domain H, and Domain I.

As used herein, Domain A begins at an amino acid position within 1-10 and ends at an amino acid position within 35-45 of SEQ ID NO: 5. Based on a preferred analysis of a local alignment generated using the Smith-Waterman algorithm, in an embodiment Domain A has been identified as amino acids 1-40 of SEQ ID NO: 5

As used herein, Domain E begins at an amino acid position within 442-452 and ends at an amino acid position within 502-512 of SEQ ID NO: 5. Based on a preferred analysis of a local alignment generated using the Smith-Waterman algorithm, in an embodiment Domain E has been identified as amino acids 447-507 of SEQ ID NO: 5.

As used herein, Domain G begins at an amino acid position within 660-670 and ends at an amino acid position within 817-827 of SEQ ID NO: 5. Based on a preferred analysis of a local alignment generated using the Smith-Waterman algorithm, in an embodiment Domain G has been identified as amino acids 665-822 of SEQ ID NO: 5.

As used herein, Domain B begins at an amino acid position within 36-46 and ends at an amino acid position within 71-81 of SEQ ID NO: 5. Based on a preferred analysis of a local alignment generated using the Smith-Waterman algorithm, in an embodiment Domain B has been identified as amino acids 41-76 of SEQ ID NO: 5.

As used herein, Domain C begins at an amino acid position within 72-82 and ends at an amino acid position within 223-233 of SEQ ID NO: 5. Based on an analysis of a local alignment generated using the Smith-Waterman algorithm, in an embodiment Domain C has been identified as amino acids 77-228 of SEQ ID NO: 5

As used herein, Domain D begins at an amino acid position within 224-234 and ends at an amino acid position within 441-451 of SEQ ID NO: 5. Based on an analysis of a local alignment generated using the Smith-Waterman algorithm, in an embodiment Domain D has been identified as amino acids 229-446 of SEQ ID NO: 5.

As used herein, Domain F begins at an amino acid position within 534-544 and ends at an amino acid position within 643-653 of SEQ ID NO: 5. Based on a preferred analysis of a local alignment generated using the Smith-Waterman algorithm, in an embodiment Domain F has been identified as amino acids 539-648 of SEQ ID NO: 5.

As used herein, Domain H begins at an amino acid position within 818-828 and ends at an amino acid position within 916-926 of SEQ ID NO: 5. Based on a preferred analysis of a local alignment generated using the Smith-Waterman algorithm, in an embodiment Domain H has been identified as amino acids 823-921 of SEQ ID NO: 5.

As used herein, Domain I begins at an amino acid position within 917-927 and ends at an amino acid position within 1057-1067 of SEQ ID NO: 5. Based on a preferred analysis of a local alignment generated using the Smith-Waterman algorithm, in an embodiment Domain I has been identified as amino acids 922-1062 of SEQ ID NO: 5.

The activity of each OMNI-79 nuclease domain is described herein, with each domain activity providing aspects of the advantageous features of the nuclease.

Specifically, OMNI-79 Domain A, Domain E, and Domain G form a structural unit of the CRISPR OMNI-79 nuclease which contains a nuclease active site that participates in DNA strand cleavage. The structural unit formed by Domain A, Domain E, and Domain G cleaves a DNA strand which a guide RNA molecule binds at a DNA target site.

Domain B is involved in initiating DNA cleavage activity upon the binding of OMNI-79 CRISPR nuclease to a target a DNA site.

Domain C and Domain D bind a guide RNA molecule and participate in providing specificity for target site recognition. More specifically, Domain C and Domain D are involved in sensing a DNA target site, with Domain D involved in regulating the activation of a nuclease domain (e.g. Domain F), and Domain C involved in locking the nuclease domain at the target site. Accordingly, Domain C and Domain D participate in controlling cleavage of off-target sequences.

Domain F contains a nuclease active site that participates in DNA strand cleavage. Domain F cleaves a DNA strand that is displaced by a guide RNA molecule binding at a double-stranded DNA target site.

Domain H also participates in the recognition of guide RNA molecules or complexes (e.g. binding regions in tracrRNA molecules, crRNA:tracrRNA complexes, or sgRNA scaffolds).

Domain I is involved in providing PAM site specificity to OMNI-79 nuclease, including aspects of PAM site interrogation and recognition. Domain I also performs topoisomerase activity.

Further description of other CRISPR nuclease domains and their general functions can be found in, inter alia, Mir et al., ACS Chem. Biol. (2019), Palermo et al., Quarterly Reviews of Biophysics (2018), Jiang and Doudna, Annual Review of Biophysics (2017), Nishimasu et al., Cell (2014) and Nishimasu et al., Cell (2015), incorporated herein by reference.

In one aspect of the invention, an amino acid sequence having similarity to an OMNI-79 domain may be utilized in the design and manufacture of a non-naturally occurring peptide, e.g. a CRISPR nuclease, such that the peptide displays the advantageous features of the OMNI-79 domain activity.

In an embodiment, such a peptide, e.g. a CRISPR nuclease, comprises an amino acid sequence that has at least 100%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence of at least one of Domain A Domain B, Domain C, Domain D, Domain E, Domain F, Domain G, Domain H, or Domain I of the OMNI-79 nuclease. In some embodiments, the identity is to at least one, at least two, at least three, at least four, at least five, at least six, at least seven or at least eight amino acid sequences of Domain A, Domain B, Domain C, Domain D, Domain E, Domain F, Domain G, Domain H, or Domain I of the OMNI-79 nuclease. Each possibility represents a separate embodiment. In some embodiments the identity is to the amino acid sequence of at least one of Domain A, Domain B, Domain C, Domain D, Domain F, Domain G, Domain H, or Domain I of the OMNI-79 nuclease. In some embodiments the identity is to the amino acid sequence of at least one of Domain F and Domain G of the OMNI-79 nuclease. In some embodiments, the CRISPR nuclease comprises an amino acid sequence corresponding to amino acid sequences of Domain G and Domain F of the OMNI-79 nuclease. In some embodiments, the CRISPR nuclease comprises an amino acid sequence having at least 100%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to amino acid sequences of Domain G and Domain F of the OMNI-79 nuclease. In an embodiment, the peptide exhibits extensive amino acid variability relative to the full length OMNI-79 amino acid sequence (SEQ ID NO: 5) outside of the peptide amino acid sequence having at least 100%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence of at least one of Domain A, Domain B, Domain C, Domain D, Domain E, Domain F, Domain G, Domain H, or Domain I of the OMNI-79 nuclease. In an embodiment, the peptide comprises an intervening amino acid sequence between two domain sequences. In an embodiment, the intervening amino acid sequence is 1-10, 10-20, 20-40, 40-50 or up to 100 amino acids in length. In an embodiment, the intervening sequence is a linker sequence.

In one aspect of the invention, an amino acid sequence encoding any one of the domains of the OMNI-79 nuclease described herein in the peptide may comprise one or more amino acid substitutions relative to the original OMNI-79 domain sequence. The amino acid substitution may be a conservative substitution, i.e. substitution for an amino acid having similar chemical properties as the original amino acid. For example, a positively charged amino acid may be substituted for an alternate positively charged amino acid, e.g. an arginine residue may be substituted for a lysine residue, or a polar amino acid may be substituted for a different polar amino acid. Conservative substitutions are more tolerable, and the amino acid sequence encoding any one of the domains of the OMNI-79 nuclease may contain as many as 10% of such substitutions. The amino acid substitution may be a radical substitution, i.e. substitution for an amino acid having different chemical properties as the original amino acid. For example, a positively charged amino acid may be substituted for a negatively charged amino acid, e.g. an arginine residue may be substituted for a glutamic acid residue, or a polar amino acid may be substituted for a non-polar amino acid. The amino acid substitution may be a semi-conservative substitution, or the amino acid substitution may be to any other amino acid. The substitution may alter the activity relative to the original OMNI-79 domain function e.g. reduce catalytic nuclease activity.

According to some aspects of the invention, the disclosed compositions comprise a non-naturally occurring composition comprising a CRISPR nuclease, wherein the CRISPR nuclease comprises an amino acid sequence corresponding to the amino acid sequence of at least one of Domain A Domain B, Domain C, Domain D, Domain E, Domain F, Domain G, Domain H, or Domain I of the OMNI-79 nuclease. In some embodiments of the invention, the CRISPR nuclease comprises at least one, at least two, at least three, at least four, or at least five amino acid sequences, wherein each amino acid sequence corresponds to any one of the amino acid sequences Domain A, Domain B, Domain C, Domain D, Domain E, Domain F, Domain G, Domain H, or Domain I of the OMNI-79 nuclease. Accordingly, the CRISPR nuclease may include any combination of amino acid sequences that corresponds to any of Domain A Domain B, Domain C, Domain D, Domain E, Domain F, Domain G, Domain H, or Domain I of the OMNI-79 nuclease. In some embodiments, the amino acid sequence is at least 100-250, 250-500, 500-1000, 1000-1500, 1000-1700, or 1000-2000 amino acids in length.

### Diseases and Therapies

Certain embodiments of the invention target a nuclease to a specific genetic locus associated with a disease or disorder as a form of gene editing, method of treatment, or therapy. For example, to induce editing or knockout of a gene, a novel nuclease disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-79 nuclease to a target site is designed to contain a spacer region complementary to a region neighboring a sequence complimentary to the OMNI-79 PAM sequence “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT international Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/DISORDERS | GENE(S) |
|---|---|
| Neoplasia | PTEN; ATM; ATR; EGFR; ERBB2; ERBB3; |
| | ERBB4; Notch1; Notch2; Notch3; Notch4; |
| | AKT; AKT2; AKT3; HIF; HIF1a; HIF3a; Met; |
| | HRG; Bcl2; PPAR alpha; PPAR gamma; WT1 |
| | (Wilms Tumor); FGF Receptor Family members |
| | (5 members: 1, 2, 3, 4, 5); CDKN2a; APC; RB |
| | (retinoblastoma); MEN1; VHL; BRCA1; |
| | BRCA2; AR (Androgen Receptor); TSG101; |
| | IGF; IGF Receptor; Igf1 (4 variants); gf2 (3 |
| | variants); Igf 1 Receptor; Igf 2 Receptor; Bax; |
| | Bcl2; caspases family (9 members: 1, 2, 3, 4, 6, |
| | 7, 8, 9, 12); Kras; Apc |
| Age-related Macular | Abcr; Ccl2; Cc2; cp (ceruloplasmin); Timp3; |
| Degeneration | cathepsinD; Vldlr; Ccr2 |
| Schizophrenia | Neuregulin1 (Nrg1); Erb4 (receptor for |
| | Neuregulin); Complexin1 (Cp1x1); Tph1 |
| | Tryptophan hydroxylase; Tph2 Tryptophan |
| | hydroxylase 2; Neurexin 1; GSK3; GSK3a; |
| | GSK3b |
| Neurological, Neuro | 5-HTT (S1c6a4); COMT; DRD (Drd1a); |
| degenerative, and | SLC6A3; DAOA; DTNBP1; Dao (Dao1) |
| Movement Disorders | |
| Trinucleotide Repeat | HTT (Huntington's Dx); SBMA/SMAX1/AR |
| Disorders | (Kennedy's Dx); FXN/X25 (Friedrich's Ataxia); |
| | ATX3 (Machado-Joseph's Dx); ATXN1 and |
| | ATXN2 (spinocerebellar ataxias); DMPK |
| | (myotonic dystrophy); Atrophin-1 and Atn1 |
| | (DRPLA Dx); CBP (Creb-BP-global |
| | instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); |
| Disorders | nicastrin (Ncstn); PEN-2 |
| Others | Nos1; Parp1; Nat1; Nat2 |
| Prion related disorders | Prp |
| ALS | SOD1; ALS2; STEX; FUS; TARDBP; VEGF |
| | (VEGF-a; VEGF-b; VEGF-c) |
| Addiction | Prkce (alcohol); Drd2; Drd4; ABAT (alcohol); |
| | GRIA2; Grm5; Grin1; Htr1b; Grin2a; Drd3; |
| | Pdyn; Gria1 (alcohol) |
| Autism | Mecp2; BZRAP1; MDGA2; Sema5A; Neurexin |
| | 1; Fragile X (FMR2 (AFF2); FXR1; FXR2; |
| | Mglur5) |
| Alzheimer's Disease | E1; CHIP; UCH; UBB; Tau; LRP; PICALM; |
| | Clusterin; PS1; SORL1; CR1; Vldlr; Uba1; |
| | Uba3; CHIP28 (Aqp1, Aquaporin 1); |
| | Uchl1; Uchl3; APP |
| Inflammation | IL-10; IL-1 (IL-1a; IL-1b); IL-13; IL-17 (IL-17a |
| | (CTLA8); IL-17b; IL-17c; IL-17d; IL-17f); II- |
| | 23; Cx3cr1; ptpn22; TNFa; NOD2/CARD15 for |
| | IBD; IL-6; IL-12 (IL-12a; IL-12b); CTLA4; |
| | Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, |
| diseases and disorders | PKLR, PK1, NT5C3, UMPH1, PSN1, |
| | RHAG, RH50A, NRAMP2, SPTB, ALAS2, |
| | ANH1, ASB, ABCB7, ABC7, ASAT); Bare |
| | lymphocyte syndrome (TAPBP, TPSN, |
| | TAP2, ABCB3, PSF2, RING11, MHC2TA, |
| | C2TA, RFX5, RFXAP, RFX5), Bleeding |
| | disorders (TBXA2R, P2RX1, P2X1); Factor |
| | H and factor H-like 1 (HF1, CFH, HUS); |
| | Factor V and factor VIII (MCFD2); Factor |
| | VII deficiency (F7); Factor X deficiency |
| | (F10); Factor XI deficiency (F11); Factor |
| | XII deficiency (F12, HAF); Factor XIIIA |
| | deficiency (F13A1, F13A); Factor XIIIB |
| | deficiency (F13B); Fanconi anemia |
| | (FANCA, FACA, FA1, FA, FAA, FAAP95, |
| | FAAP90, FLJ34064, FANCB, FANCC, |
| | FACC, BRCA2, FANCD1, FANCD2, |
| | FANCD, FACD, FAD, FANCE, FACE, |
| | FANCF, XRCC9, FANCG, BRIP1, BACH1, |
| | FANCJ, PHF9, FANCL, FANCM, |
| | KIAA1596); Hemophagocytic |
| | lymphohistiocytosis disorders (PRF1, |
| | HPLH2, UNC13D, MUNC13-4, HPLH3, |
| | HLH3, FHL3); Hemophilia A (F8, F8C, |
| | HEMA); Hemophilia B (F9, HEMB), |
| | Hemorrhagic disorders (PI, ATT, F5); |
| | Leukocyde deficiencies and disorders |
| | (ITGB2, CD18, LCAMB, LAD, EIF2B1, |
| | EIF2BA, EIF2B2, EIF2B3, EIF2B5, |
| | LVWM, CACH, CLE, EIF2B4); Sickle cell |
| | anemia (HBB); Thalassemia (HBA2, HBB, |
| | HBD, LCRB, HBA1) |
| Cell dysregulation and | B-cell non-Hodgkin lymphoma (BCL7A, |
| oncology diseases and | BCL7); Leukemia (TAL1, TCL5, SCL, |
| disorders | TAL2, FLT3, NBS1, NBS, ZNFN1A1, IK1, |
| | LYF1, HOXD4, HOX4B, BCR, CML, PHL, |
| | ALL, ARNT, KRAS2, RASK2, GMPS, |
| | AF10, ARHGEF12, LARG, KIAA0382, |
| | CALM, CLTH, CEBPA, CEBP, CHIC2, |
| | BTL, FLT3, KIT, PBT, LPP, NPM1, |
| | NUP214, D9S46E, CAN, CAIN, RUNX1, |
| | CBFA2, AML1, WHSC1L1, NSD3, FLT3, |
| | AF1Q, NPM1, NUMA1, ZNF145, PLZF, |
| | PML, MYL, STAT5B, AF10, CALM, |
| | CLTH, ARL11, ARLTS1, P2RX7, P2X7, |
| | BCR, CML, PHL, ALL, GRAF, NF1, |
| | VRNF, WSS, NFNS, PTPN11, PTP2C, |
| | SHP2, NS1, BCL2, CCND1, PRAD1, |
| | BCL1, TCRA, GATA1, GF1, ERYF1, |
| | NFE1, ABL1, NQO1, DIA4, NMOR1, |
| | NUP214, D9S46E, CAN, CAIN) |
| Inflammation and immune | AIDS (KIR3DL1, NKAT3, NKB1, AMB11, |
| related diseases and | KIR3DS1, IFNG, CXCL12, SDF1); |
| disorders | Autoimmune lymphoproliferative syndrome |
| | (TNFRSF6, APT1, FAS, CD95, ALPS1A); |
| | Combined immunodeficiency, (IL2RG, |
| | SCIDX1, SCIDX, IMD4); HIV-1 (CCL5, |
| | SCYA5, D17S136E, TCP228), HIV |
| | susceptibility or infection (IL10, CSIF, |
| | CMKBR2, CCR2, CMKBR5, CCCKR5 |
| | (CCR5)); Immunodeficiencies (CD3E, |
| | CD3G, AICDA, AID, HIGM2, TNFRSF5, |
| | CD40, UNG, DGU, HIGM4, TNFSF5, |
| | CD40LG, HIGM1, IGM, FOXP3, IPEX, |
| | AIID, XPID, PIDX, TNFRSF14B, TACI); |
| | Inflammation (IL-10, IL-1 (IL-1a, IL-1b), |
| | IL-13, IL-17 (IL-17a (CTLA8), IL-17b, IL- |
| | 17c, IL- 17d, IL-17f), II-23, Cx3cr1, ptpn22, |
| | TNFa, NOD2/CARD15 for IBD, IL-6, IL-12 |
| | (IL-12a, IL-12b), CTLA4, Cx3cl1); Severe |
| | combined immunodeficiencies |
| | (SCIDs)(JAK3, JAKL, DCLRE1C, |
| | ARTEMIS, SCIDA, RAG1, RAG2, ADA, |
| | PTPRC, CD45, LCA, IL7R, CD3D, T3D, |
| | IL2RG, SCIDX1, SCIDX, IMD4) |
| Metabolic, liver, kidney | Amyloid neuropathy (TTR, PALB); |
| and protein diseases and | Amyloidosis (APOA1, APP, AAA, CVAP, |
| disorders | AD1, GSN, FGA, LYZ, TTR, PALB); |
| | Cirrhosis (KRT18, KRT8, CIRH1A, NAIC, |
| | TEX292, KIAA1988); Cystic fibrosis |
| | (CFTR, ABCC7, CF, MRP7); Glycogen |
| | storage diseases (SLC2A2, GLUT2, G6PC, |
| | G6PT, G6PT1, GAA, LAMP2, LAMPB, |
| | AGL, GDE, GBE1, GYS2, PYGL, PFKM); |
| | Hepatic adenoma, 142330 (TCF1, HNF1A, |
| | MODY3), Hepatic failure, early onset, and |
| | neurologic disorder (SCOD1, SCO1), |
| | Hepatic lipase deficiency (LIPC), |
| | Hepatoblastoma, cancer and carcinomas |
| | (CTNNB1, PDGFRL, PDGRL, PRLTS, |
| | AXIN1, AXIN, CTNNB1, TP53, P53, LFS1, |
| | IGF2R, MPRI, MET, CASP8, MCH5; |
| | Medullary cystic kidney disease (UMOD, |
| | HNFJ, FJHN, MCKD2, ADMCKD2); |
| | Phenylketonuria (PAH, PKU1, QDPR, |
| | DHPR, PTS); Polycystic kidney and hepatic |
| | disease (FCYT, PKHD1, ARPKD, PKD1, |
| | PKD2, PKD4, PKDTS, PRKCSH, G19P1, |
| | PCLD, SEC63) |
| Muscular/Skeletal | Becker muscular dystrophy (DMD, BMD, |
| diseases and disorders | MYF6), Duchenne Muscular Dystrophy |
| | (DMD, BMD); Emery-Dreifuss muscular |
| | dystrophy (LMNA, LMN1, EMD2, FPLD, |
| | CMD1A, HGPS, LGMD1B, LMNA, LMN1, |
| | EMD2, FPLD, CMD1A); |
| | Facioscapulohumeral muscular dystrophy |
| | (FSHMD1A, FSHD1A); Muscular dystrophy |
| | (FKRP, MDC1C, LGMD2I, LAMA2, |
| | LAMM, LARGE, KIAA0609, MDC1D, |
| | FCMD, TTID, MYOT, CAPN3, CANP3, |
| | DYSF, LGMD2B, SGCG, LGMD2C, |
| | DMDA1, SCG3, SGCA, ADL, DAG2, |
| | LGMD2D, DMDA2, SGCB, LGMD2E, |
| | SGCD, SGD, LGMD2F, CMD1L, TCAP, |
| | LGMD2G, CMD1N, TRIM32, HT2A, |
| | LGMD2H, FKRP, MDC1C, LGMD2I, TTN, |
| | CMD1G, TMD, LGMD2J, POMT1, CAV3, |
| | LGMD1C, SEPN1, SELN, RSMD1, PLEC1, |
| | PLTN, EBS1); Osteopetrosis (LRP5, |
| | BMND1, LRP7, LR3, OPPG, VBCH2, |
| | CLCN7, CLC7, OPTA2, OSTM1, GL, |
| | TCIRG1, TIRC7, OC116, OPTB1); |
| | Muscular atrophy (VAPB, VAPC, ALS8, |
| | SMN1, SMAl, SMA2, SMA3, SMA4, |
| | BSCL2, SPG17, GARS, SMAD1, CMT2D, |
| | HEXB, IGHMBP2, SMUBP2, CATF1, |
| | SMARD1) |
| Dermatological diseases | Albinisim (TYR, OCA2, TYRP1, SLC45A2, |
| and disorders | LYST), Ectodermal dysplasias (EDAR, |
| | EDARADD, WNT10A), Ehlers-Danlos |
| | syndrome (COL5A1, COL5A2, COL1A1, |
| | COL1A2, COL3A1, TNXB, ADAMTS2, |
| | PLOD1, FKBP14), Ichthyosis-associated |
| | disorders (FLG, STS, TGM1, |
| | ALOXE3/ALOX12B, KRT1, KRT10, |
| | ABCA12, KRT2, GJB2, TGM1, ABCA12, |
| | CYP4F22, ALOXE3, CERS3, NSHDL, |
| | EBP, MBTPS2, GJB2, SPINK5, AGHD5, |
| | PHYH, PEX7, ALDH3A2, ERCC2, ERCC3, |
| | GFT2H5, GBA), Incontinentia pigmenti |
| | (IKBKG, NEMO), Tuberous sclerosis |
| | (TSC1, TSC2), Premature aging syndromes |
| | (POLR3A, PYCR1, LMNA, POLD1, WRN, |
| | DMPK) |
| Neurological and Neuronal | ALS (SOD1, ALS2, STEX, FUS, TARDBP, |
| diseases and disorders | VEGF (VEGF-a, VEGF-b, VEGF-c); |
| | Alzheimer disease (APP, AAA, CVAP, |
| | AD1, APOE, AD2, PSEN2, AD4, STM2, |
| | APBB2, FE65L1, NOS3, PLAU, URK, |
| | ACE, DCP1, ACE1, MPO, PACIP1, |
| | PAXIP1L, PTIP, A2M, BLMH, BMH, |
| | PSEN1, AD3); Autism (Mecp2, BZRAP1, |
| | MDGA2, Sema5A, Neurexin 1, GLO1, |
| | MECP2, RTT, PPMX, MRX16, MRX79, |
| | NLGN3, NLGN4, KIAA1260, AUTSX2); |
| | Fragile X Syndrome (FMR2, FXR1, FXR2, |
| | mGLUR5); Huntington's disease and disease |
| | like disorders (HD, IT15, PRNP, PRIP, |
| | JPH3, JP3, HDL2, TBP, SCA17); Parkinson |
| | disease (NR4A2, NURR1, NOT, TINUR, |
| | SNCAIP, TBP, SCA17, SNCA, NACP, |
| | PARK1, PARK4, DJ1, PARK7, LRRK2, |
| | PARK8, PINK1, PARK6, UCHL1, PARK5, |
| | SNCA, NACP, PARK1, PARK4, PRKN, |
| | PARK2, PDJ, DBH, NDUFV2); Rett |
| | syndrome (MECP2, RTT, PPMX, MRX16, |
| | MRX79, CDKL5, STK9, MECP2, RTT, |
| | PPMX, MRX16, MRX79, x-Synuclein, |
| | DJ-1); Schizophrenia (Neuregulin1 (Nrg1), |
| | Erb4 (receptor for Neuregulin), Complexin1 |
| | (Cplx1), Tph1 Tryptophan hydroxylase, |
| | Tph2, Tryptophan hydroxylase 2, Neurexin |
| | 1, GSK3, GSK3a, GSK3b, 5-HTT (Slc6a4), |
| | COMT, DRD (Drd1a), SLC6A3, DAOA, |
| | DTNBP1, Dao (Dao1)); Secretase Related |
| | Disorders (APH-1 (alpha and beta), |
| | Presenilin (Psen1), nicastrin, (Ncstn), PEN- |
| | 2, Nos1, Parp1, Natl, Nat2); Trinucleotide |
| | Repeat Disorders (HTT (Huntington's Dx), |
| | SBMA/SMAX1/AR (Kennedy's Dx), |
| | FXN/X25 (Friedrich's Ataxia), ATX3 |
| | (Machado-Joseph's Dx), ATXN1 and |
| | ATXN2 (spinocerebellar ataxias), DMPK |
| | (myotonic dystrophy), Atrophin-1 and Atn1 |
| | (DRPLA Dx), CBP (Creb-BP-global |
| | instability), VLDLR (Alzheimer's), Atxn7, |
| | Atxn10) |
| Ocular diseases and | Age-related macular degeneration (Abcr, |
| disorders | Ccl2, Cc2, cp (ceruloplasmin), Timp3, |
| | cathepsinD, Vldlr, Ccr2); Cataract (CRYAA, |
| | CRYA1, CRYBB2, CRYB2, PITX3, |
| | BFSP2, CP49, CP47, CRYAA, CRYA1, |
| | PAX6, AN2, MGDA, CRYBA1, CRYB1, |
| | CRYGC, CRYG3, CCL, LIM2, MP19, |
| | CRYGD, CRYG4, BFSP2, CP49, CP47, |
| | HSF4, CTM, HSF4, CTM, MIP, AQP0, |
| | CRYAB, CRYA2, CTPP2, CRYBB1, |
| | CRYGD, CRYG4, CRYBB2, CRYB2, |
| | CRYGC, CRYG3, CCL, CRYAA, CRYA1, |
| | GJA8, CX50, CAE1, GJA3, CX46, CZP3, |
| | CAE3, CCM1, CAM, KRIT1); Corneal |
| | clouding and dystrophy (APOA1, TGFBI, |
| | CSD2, CDGG1, CSD, BIGH3, CDG2, |
| | TACSTD2, TROP2, M1S1, VSX1, RINX, |
| | PPCD, PPD, KTCN, COL8A2, FECD, |
| | PPCD2, PIP5K3, CFD); Cornea plana |
| | congenital (KERA, CNA2); Glaucoma |
| | (MYOC, TIGR, GLC1A, JOAG, GPOA, |
| | OPTN, GLC1E, FIP2, HYPL, NRP, |
| | CYP1B1, GLC3A, OPA1, NTG, NPG, |
| | CYP1B1, GLC3A); Leber congenital |
| | amaurosis (CRB1, RP12, CRX, CORD2, |
| | CRD, RPGRIP1, LCA6, CORD9, RPE65, |
| | RP20, AIPL1, LCA4, GUCY2D, GUC2D, |
| | LCA1, CORD6, RDH12, LCA3); Macular |
| | dystrophy (ELOVL4, ADMD, STGD2, |
| | STGD3, RDS, RP7, PRPH2, PRPH, AVMD, |
| | AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

It is understood that where a numerical range is recited herein, the present invention contemplates each integer between, and including, the upper and lower limits, unless otherwise stated.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, in Irons, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers, A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate. Each of the RNA sequences described herein may comprise one or more nucleotide analogs.

As used herein, the following nucleotide identifiers listed in Table C below are used to represent a referenced nucleotide base(s):

TABLE C
Nucleotide identifiers

| Nucleotide | | | | |
|---|---|---|---|---|
| reference | Base(s) represented | | | |
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of a targeting RNA molecule that can form a complex with a CRISPR nuclease with the targeting sequence serving as the targeting portion of the CRISPR complex. When the molecule having the targeting sequence is present contemporaneously with the CRISPR molecule, the RNA molecule is capable of targeting the CRISPR nuclease to the specific target sequence. Each possibility represents a separate embodiment. A targeting RNA molecule can be custom designed to target any desired sequence.

The term “targets” as used herein, refers to preferential hybridization of a targeting sequence or a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

In the context of targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is affected by a complex of an RNA molecule and the CRISPR nuclease as a result of hybridization with the target sequence, i.e. on-target hybridization.

As used herein the term “wild type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion) compared to the CRISPR nuclease of any of the CRISPR nucleases indicated in Tables 1a-1b.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

As used herein the term “amino acid” includes natural and/or unnatural or synthetic amino acids, including glycine and both the D or I, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The term “PAM” as used herein refers to a nucleotide sequence of a target DNA located in proximity to the targeted DNA sequence and recognized by the CRISPR nuclease. The PAM sequence may differ depending on the nuclease identity.

The term “mutation disorder” or “mutation disease” as used herein refers to any disorder or disease that is related to dysfunction of a gene caused by a mutation. A dysfunctional gene manifesting as a mutation disorder contains a mutation in at least one of its alleles and is referred to as a “disease-associated gene.” The mutation may be in any portion of the disease-associated gene, for example, in a regulatory, coding, or non-coding portion. The mutation may be any class of mutation, such as a substitution, insertion, or deletion. The mutation of the disease-associated gene may manifest as a disorder or disease according to the mechanism of any type of mutation, such as a recessive, dominant negative, gain-of-function, loss-of-function, or a mutation leading to haploinsufficiency of a gene product.

A skilled artisan will appreciate that embodiments of the present invention disclose RNA molecules capable of complexing with a nuclease, e.g. a CRISPR nuclease, such as to associate with a target genomic DNA sequence of interest next to a protospacer adjacent motif (PAM). The nuclease then mediates cleavage of target DNA to create a double-stranded break within the protospacer.

In embodiments of the present invention, a CRISPR nuclease and a targeting molecule form a CRISPR complex that binds to a target DNA sequence to effect cleavage of the target DNA sequence. A CRISPR nuclease may form a CRISPR complex comprising the CRISPR nuclease and RNA molecule without a further, separate tracrRNA molecule. Alternatively, CRISPR nucleases may form a CRISPR complex between the CRISPR nuclease, an RNA molecule, and a tracrRNA molecule.

The term “protein binding sequence” or “nuclease binding sequence” refers to a sequence capable of binding with a CRISPR nuclease to form a CRISPR complex. A skilled artisan will understand that a tracrRNA capable of binding with a CRISPR nuclease to form a CRISPR complex comprises a protein or nuclease binding sequence.

An “RNA binding portion” of a CRISPR nuclease refers to a portion of the CRISPR nuclease which may bind to an RNA molecule to form a CRISPR complex, e.g. the nuclease binding sequence of a tracrRNA molecule. An “activity portion” or “active portion” of a CRISPR nuclease refers to a portion of the CRISPR nuclease which effects a double strand break in a DNA molecule, for example when in complex with a DNA-targeting RNA molecule.

An RNA molecule may comprise a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

In embodiments of the present invention, the targeting molecule may further comprise the sequence of a tracrRNA molecule. Such embodiments may be designed as a synthetic fusion of the guide portion of the RNA molecule (gRNA or crRNA) and the trans-activating crRNA (tracrRNA), together forming a single guide RNA (sgRNA). (See Jinek et al., Science (2012)). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via base pairing and may be advantageous in certain applications of the invention described herein.

In embodiments of the present invention an RNA molecule may comprise a “nexus” region and/or “hairpin” regions which may further define the structure of the RNA molecule. (See Briner et al., Molecular Cell (2014)).

As used herein, the term “direct repeat sequence” refers to two or more repeats of a specific amino acid sequence of nucleotide sequence.

As used herein, an RNA sequence or molecule capable of “interacting with” or “binding” with a CRISPR nuclease refers to the RNA sequence or molecules ability to form a CRISPR complex with the CRISPR nuclease.

As used herein, the term “operably linked” refers to a relationship (i.e. fusion, hybridization) between two sequences or molecules permitting them to function in their intended manner. In embodiments of the present invention, when an RNA molecule is operably linked to a promoter, both the RNA molecule and the promotor are permitted to function in their intended manner.

As used herein, the term “heterologous promoter” refers to a promoter that does not naturally occur together with the molecule or pathway being promoted.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

### Nuclear Localization Sequences

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known. Non-limiting examples of NLSs include an NLS sequence derived from: the SV40 virus large T-antigen, nucleoplasmin, c-myc, the hRNPA1 M9 NLS, the IBB domain from importin-alpha, myoma T protein, human p53, mouse c-abl IV, influenza vims NS1, Hepatitis virus delta antigen, mouse Mx1 protein, human poly(ADP-ribose) polymerase, and the steroid hormone receptors (human) glucocorticoid.

### Delivery

The CRISPR nuclease or CRISPR compositions described herein may be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-O-methyl (M), 2′-O-methyl, 3′phosphorothioate (MS) or 2-O-methyl, 3′thioPACE (MSP), pseudouridine, and 1-methyl pseudo-uridine. Each possibility represents a separate embodiment of the present invention.

The CRISPR nucleases and/or polynucleotides encoding same described herein, and optionally additional proteins (e.g., ZFPs, TALENs, transcription factors, restriction enzymes) and/or nucleotide molecules such as guide RNA may be delivered to a target cell by any suitable means. The target cell may be any type of cell e.g., eukaryotic or prokaryotic, in any environment e.g., isolated or not, maintained in culture, in vitro, ex vivo, in vivo or in p/anta.

In some embodiments, the composition to be delivered includes mRNA of the nuclease and RNA of the guide. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. In some embodiments, the composition to be delivered includes mRNA of the nuclease, DNA-targeting RNA and the tracrRNA. In some embodiments, the composition to be delivered includes mRNA of the nuclease, DNA-targeting RNA and the tracrRNA and a donor template. In some embodiments, the composition to be delivered includes the CRISPR nuclease DNA-targeting RNA and the tracrRNA. In some embodiments, the composition to be delivered includes the CRISPR nuclease, DNA-targeting RNA and the tracrRNA and a donor template for gene editing via, for example, homology directed repair.

Any suitable viral vector system may be used to deliver RNA compositions. Conventional viral and non-viral based gene transfer methods can be used to introduce nucleic acids and/or CRISPR nuclease in cells (e.g., mammalian cells, plant cells, etc.) and target tissues. Such methods can also be used to administer nucleic acids encoding and/or CRISPR nuclease protein to cells in vitro. In certain embodiments, nucleic acids and/or CRISPR nuclease are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science (1992); Nabel and Felgner, TIBTECH (1993); Mitani and Caskey, TIBTECH (1993); Dillon, TIBTECH (1993); Miller, Nature (1992); Van Brunt, Biotechnology (1988); Vigne et al., Restorative Neurology and Neuroscience 8:35-36 (1995); Kremer and Perricaudet, British Medical Bulletin (1995); Haddada et al., Current Topics in Microbiology and Immunology (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., Cancer Gene Ther. (1995); Behr et al., Bioconjugate Chem. (1994); Remy et al., Bioconjugate Chem. (1994); Gao and Huang, Gene Therapy (1995); Ahmad and Allen, Cancer Res., (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al., Nature Biotechnology (2009)).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, recombinant retroviral, lentivirus, adenoviral, adeno-associated, vaccinia and herpes simplex virus vectors for gene transfer. However, an RNA virus is preferred for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991): PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. In some embodiments, delivery of mRNA in-vivo and ex-vivo, and RNPs delivery may be utilized.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney, “Culture of Animal Cells, A Manual of Basic Technique and Specialized Applications (6th edition, 2010)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodoptera fugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in-vitro or can be introduced into a mammal (such as the donor of the cells) where they will engraft in the bone marrow. Methods for differentiating CD34+ cells in vitro into clinically important immune cell types using cytokines such a GM-CSF, IFN-gamma. and TNF-alpha are known (as a non-limiting example see, Inaba et al., J. Exp. Med. (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (as a non-limiting example see Inaba et al., J. Exp. Med. (1992)). Stem cells that have been modified may also be used in some embodiments.

Notably, any one of the CRISPR nucleases described herein may be suitable for genome editing in post-mitotic cells or any cell which is not actively dividing, e.g., arrested cells. Examples of post-mitotic cells which may be edited using a CRISPR nuclease of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### DNA Repair by Homologous Recombination

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987): Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) a RNA molecule comprising a guide sequence to affect a double strand break in a gene prior to repair and (2) a donor RNA template for repair, the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by recombinant viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes 1-111 (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828, 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells—A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization—A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

CRISPR repeat (crRNA), transactivating crRNA (tracrRNA), nuclease polypeptide, and PAM sequences were predicted from different metagenomic databases of sequences of environmental samples. The list of bacterial species/strains from which the CRISPR repeat, tracRNA sequence, and nucleases polypeptide sequence were predicted is provided in Tables 1a-1b.

### Construction of OMNI Nuclease Polypeptides

For construction of OMNI nuclease polypeptides, the open reading frame of several identified OMNI nucleases (OMNIs) were codon optimized for human cell line expression. The ORF was cloned into the bacterial plasmid pb-NNC and into the mammalian plasmid pmOMNI (Table 4).

Prediction and Construction of sgRNA

For each OMNI the sgRNA was predicted by detection of the CRISPR repeat array sequence (crRNA) and a trans-activating crRNA (tracrRNA) in the respective bacterial genome. The native pre-mature crRNA and tracrRNA sequences were connected in-silico with tetra-loop ‘gaaa’ and the secondary structure elements of the duplex were predicted by using an RNA secondary structure prediction tool.

The predicted secondary structures of the full duplex RNA elements (crRNA-tracrRNA chimera) was used for identification of possible tracr sequences for the design of a sgRNA having various versions for each OMNI nuclease. By shortening the duplex at the upper stem at different locations, the crRNA and tracrRNA were connected with tetra-loop ‘gaaa’, thereby generating possible sgRNA scaffolds (sgRNA designs of all OMNIs are listed in Tables 2a-2d). At least two versions of possible designed scaffolds for each OMNI were synthesized and connected downstream to a 22 nt universal unique spacer sequence (T2, SEQ ID NO: 41) and cloned into a bacterial expressing plasmid under a constitutive promoter and into a mammalian expression plasmid under a U6 promoter (pbSGR2 and pmGuide, respectively, Table 4).

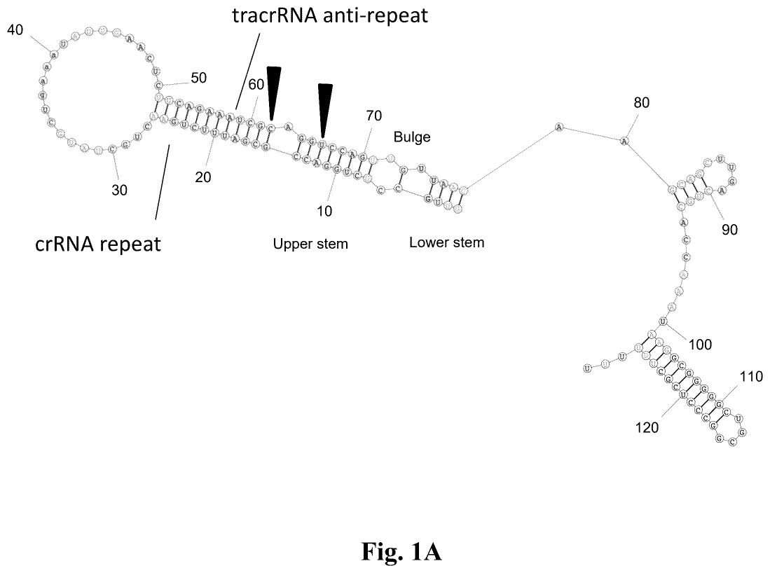

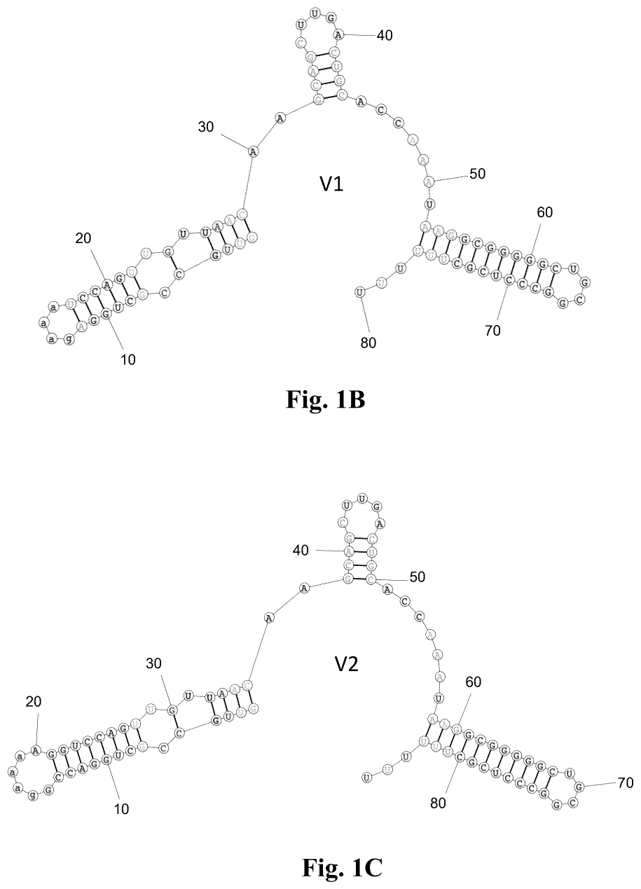

In order to overcome potential transcriptional and structural constraints and to assess the plasticity of the sgRNA scaffold in the human cellular environmental context, several versions of the sgRNA were tested. In each case the modifications represent small variations in the nucleotide sequence of the possible sgRNA (FIG. 1C, Tables 2a-2d).

T1-
(SEQ ID NO: 241)
GGTGCGGTTCACCAGGGTGTCG
T2-
(SEQ ID NO: 242)
GGAAGAGCAGAGCCTTGGTCTC

### In-Vitro Depletion Assay by TXTL

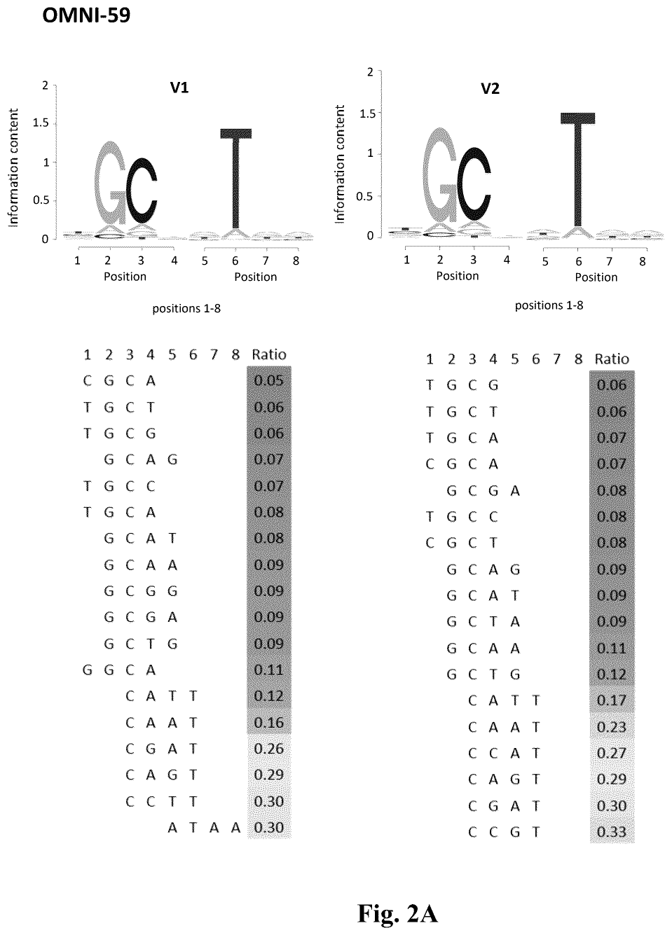

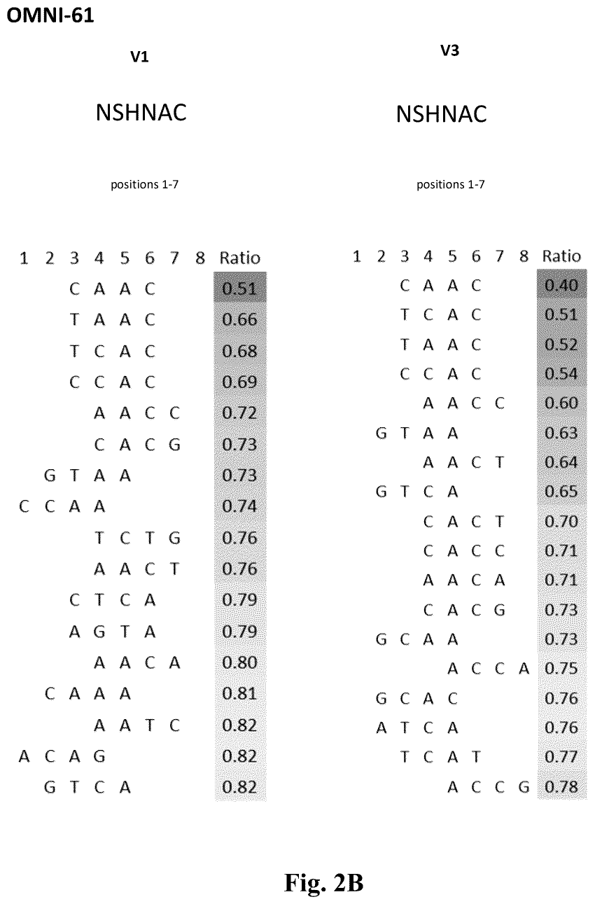

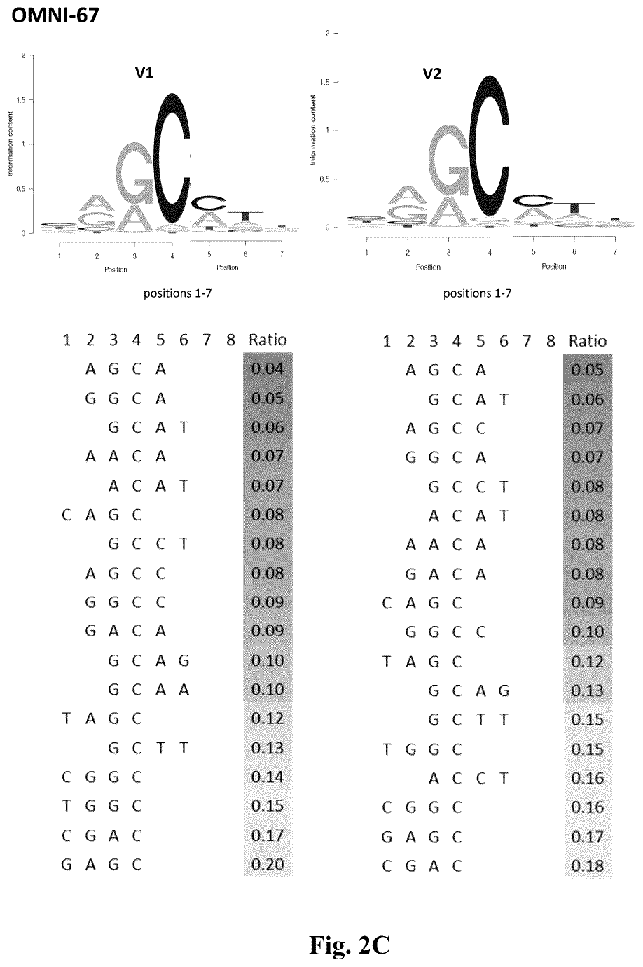

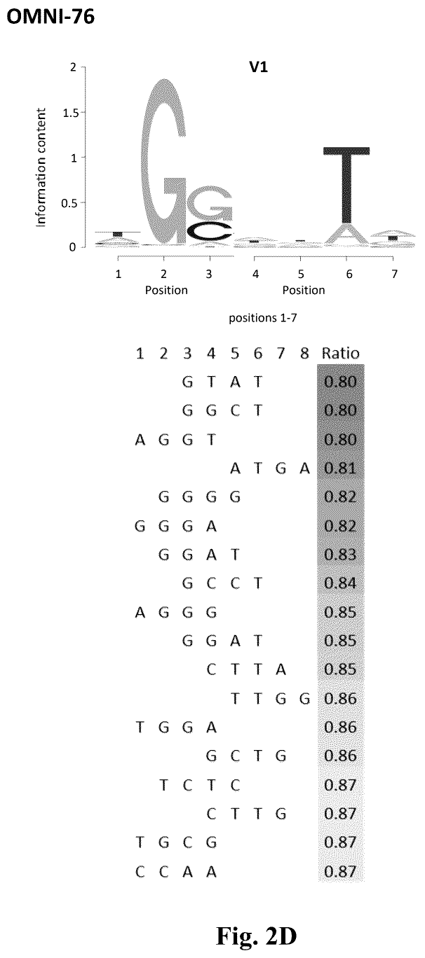

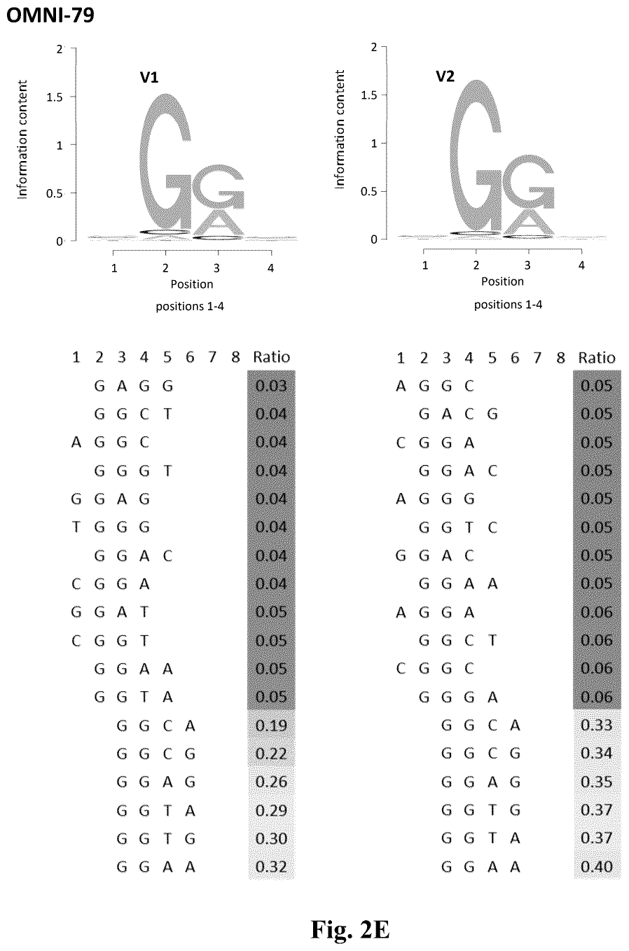

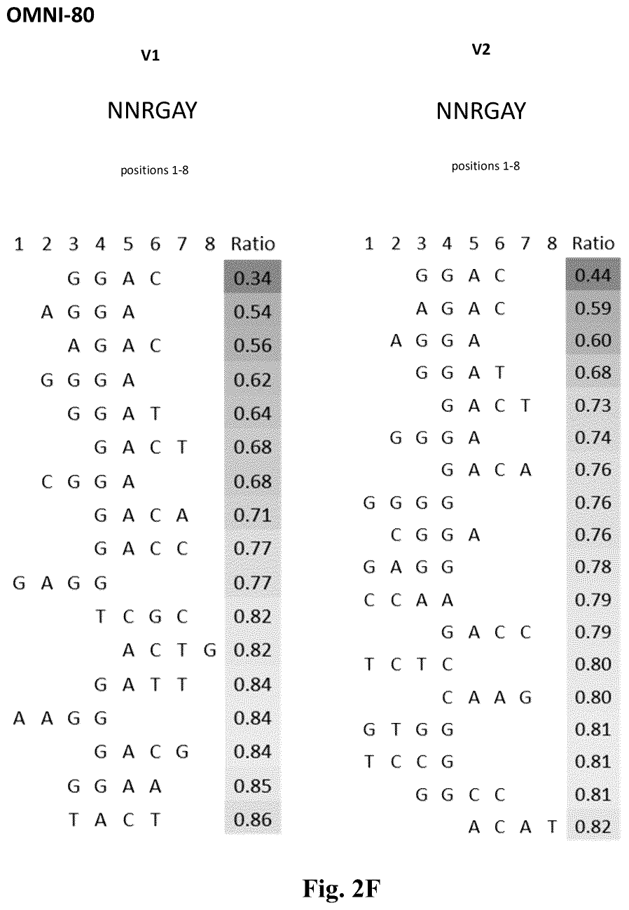

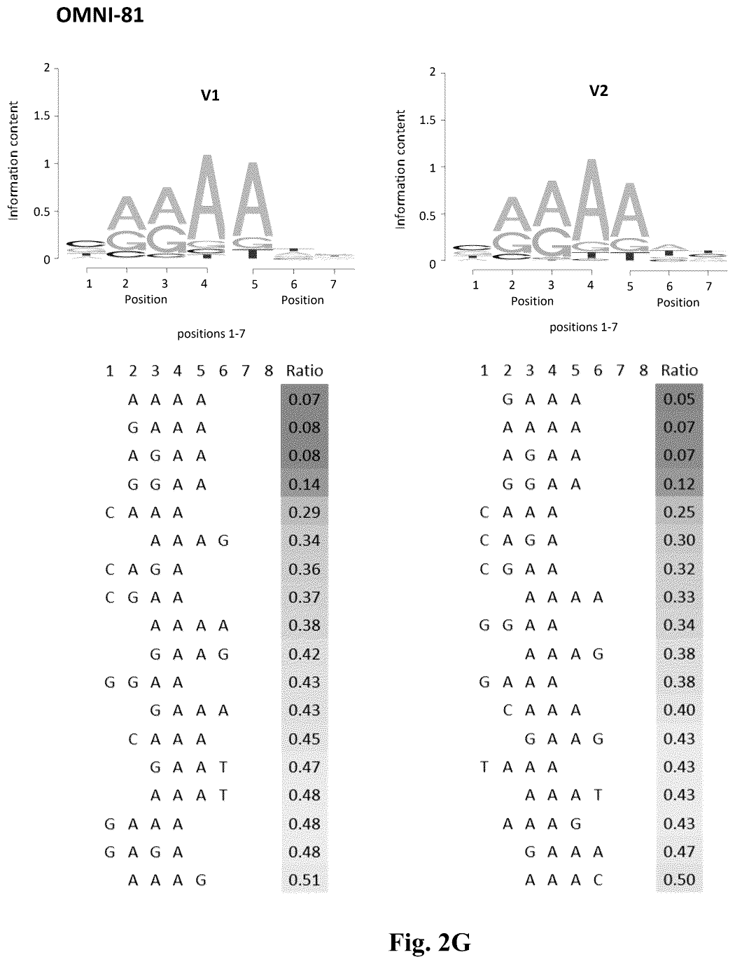

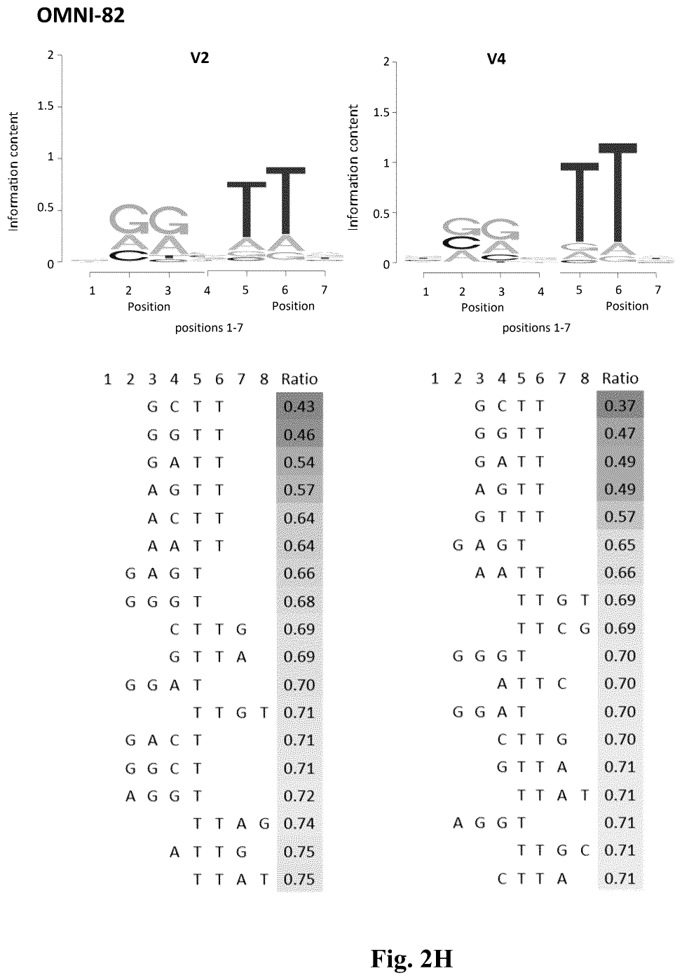

Depletion of PAM sequences in-vitro was followed by Maxwell et al, Methods. 2018. Briefly, linear DNA expressing the OMNI nucleases and an sgRNA under T7 promoter were added to a TXTL mix (Arbor Bioscience) together with a linear construct expressing T7 polymerase. RNA expression and protein translation by the TXTL mix result in the formation of the RNP complex. Since linear DNA was used, Chi6 sequences, a RecBCD inhibitor, were added to protect the DNA from degradation. The sgRNA spacer is designed to target a library of plasmids containing the targeting protospacer (pbPOS T2 library, Table 4) flanked by an 8N randomized set of potential PAM sequences. Depletion of PAM sequences from the library was measured by high-throughput sequencing upon using PCR to add the necessary adapters and indices to both the cleaved library and to a control library expressing a non-targeting gRNA (T1). Following deep sequencing, the in-vitro activity was confirmed by the fraction of the depleted sequences having the same PAM sequence relative to their occurrence in the control by the OMNI nuclease indicating functional DNA cleavage by an in-vitro system (FIGS. 2A-2H, Tables 3a-3c).

### Nuclease Expression in a Mammalian Cells

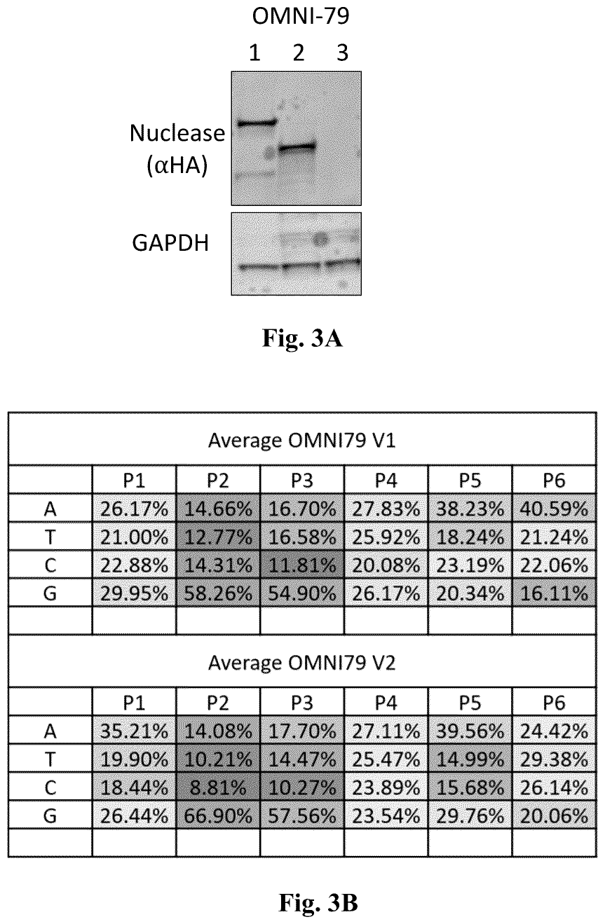

First, expression of each of the optimized DNA sequences coding for OMNI nucleases in mammalian cells was validated. To this end, an expression vector coding for an HA-tagged OMNI nuclease or Streptococcus Pyogenes Cas9 (SpCas9) linked to mCherry by a P2A peptide (pmOMNI, Table 4) was introduced into Hek293T cells using the Jet-optimus™ transfection reagent (polyplus-transfection). The P2A peptide is a self-cleaving peptide which can induce the cleaving of the recombinant protein in a cell such that the OMNI nuclease and the mCherry are separated upon expression. The mCherry serves as indicator for transcription efficiency of the OMNI from expression vector. Expression of all OMNI proteins was confirmed by a western blot assay using anti-HA antibody (FIG. 3A).

### PAM Identification and Activity in Mammalian Cells

While a PAM sequence preference is considered as an inherent property of the nuclease, it may be affected, to some extent, by the cellular environment, genomic composition and genome size. Since the human cellular environment is significantly different from the bacterial environment with respect to those properties, a “fine tuning” step has been introduced to address potential differences in PAM preferences in the human cellular context. To this end, a PAM library was constructed in a human cell line. The PAM library was introduced to the cells using a viral vector (see Table 4), as a constant target sequence followed by a stretch of 6N. Upon introduction of an OMNI and an sgRNA targeting the library constant target site, NGS analysis was used to identify the edited sequences and the PAM associated with them. The enriched edited sequences were then used to define the PAM consensus. This methodology was applied to determine the optimized PAM requirements of OMNI nuclease in mammalian cells (FIG. 3B, Tables 3a-3c “mammalian refinements”). The OMNI-79 PAM is an NGG motif that is similar to the NGR PAM identified by TXTL.

### Activity in Human Cells on Endogenous Genomic Targets

OMNIs were also assayed for their ability to promote editing on specific genomic locations in human cells. To this end, for each OMNI a corresponding OMNI-P2A-mCherry expression vector (pmOMNI, Table 4) was transfected into HeLa cells together with an sgRNA designed to target a specific location in the human genome (pmGuide, Table 4). At 72 h, cells were harvested. Half of the cells were used for quantification of transfection efficiency by FACS using mCherry fluorescence as a marker. The other half of the cells were lysed, and their genomic DNA content was used to PCR amplify the corresponding putative genomic targets. Amplicons were subjected to NGS and the resulting sequences were then used to calculate the percentage of editing events in each target site. Short Insertions or deletions (indels) around the cut site are the typical outcome of repair of DNA ends following nuclease-induced DNA cleavage. The calculation of percent editing was therefore deduced from the fraction of indel-containing sequences within each amplicon.

Genomic activity of each OMNI was assessed using a panel of a minimum of 6 and up to a maximum of 31 unique sgRNAs each designed to target a different genomic location. The results of these experiments are summarized in Table 5. As can be seen in the table (column 6, “% indels”), several OMNIs exhibit significant editing levels compared to the negative control (column 9, “% editing in neg control”) in several target sites tested. OMNI-59 and OMNI-67 exhibited significant editing levels in 1/6 sites tested, OMNI-79 exhibited high and significant editing levels in 19/31 sites tested with editing activity >5%, and OMNI-81 exhibited high and significant editing levels in 6/10 sites tested.

OMNI-79 Activity with gRNA Variation

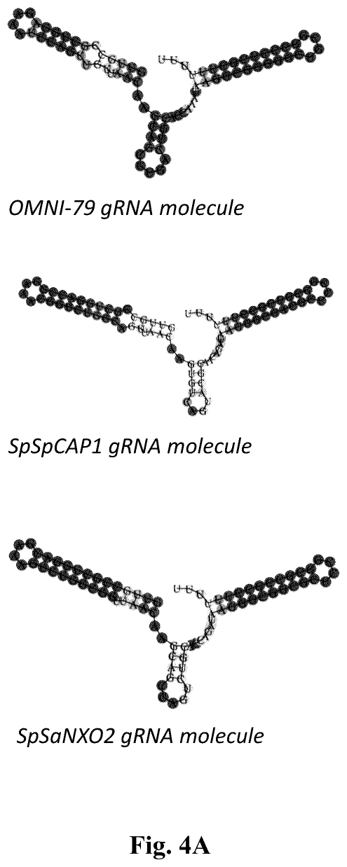

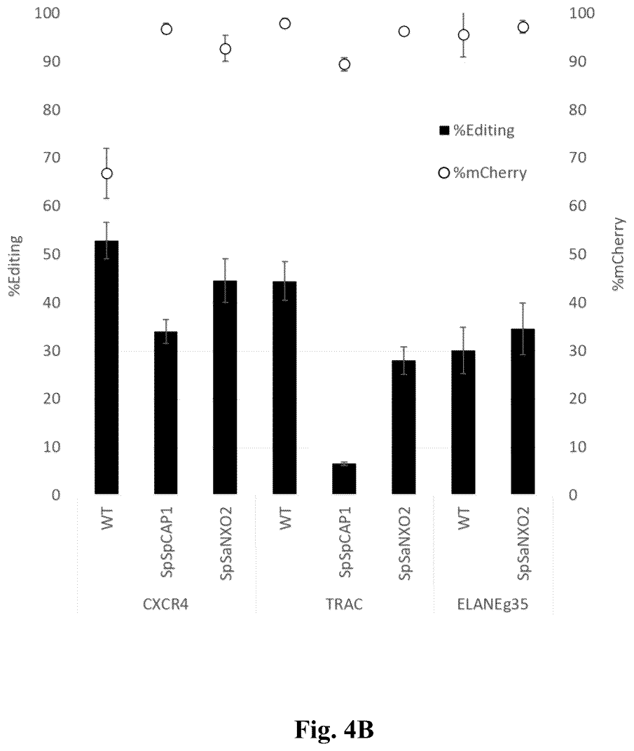

Other gRNA molecules sharing the same repeat sequence as an OMNI-79 gRNA molecule were also identified. Specifically, two gRNA molecules that share the repeat sequence and are predicted to form a similar structure were identified (FIG. 4A). We next tested the ability of OMNI-79 to function with these gRNA molecules by DNA-targeting such a complex to several genomic locations. OMNI-79 plasmid was transfected into HeLa cells together with a sgRNA molecule designed to target a specific location in the human genome (pmGuide, Table 4, spacer sequence, Table 5) designed with the native scaffold (Novosphingobium sp. SYSU G00007 “WT”) or an alternative scaffold (“SpSaNXO2” or “SpSpCAP1”). At 72 hours cells were harvested and half of the cells were used for quantification of transfection efficiency by FACS, using mCherry fluorescence as marker. The rest of the cells were lysed, and their genomic DNA content was used in a PCR reaction which amplified the corresponding putative genomic targets. Amplicons were subjected to next-generation sequencing (NGS) and the resulting sequences were then used calculate the percentage of editing events in each target site. Short insertions or deletions (indels) around the cut site are the typical outcome of repair of DNA ends following nuclease induced DNA cleavage. The calculation of % editing was therefore deduced from the fraction of indels containing sequences within each amplicon.

As can be seen in FIG. 4B, the editing level obtained by OMNI-79 using the SpSaNXO2 scaffold is comparable to the editing level obtained using the native gRNA molecule in all sites tested. The editing level obtained by OMNI-79 using the SpSpCAP1 gRNA molecule is reduced compared with the editing level obtained using the native gRNA molecule.

### OMNI-79 Activity Via AAV Delivery

OMNI-79 was subcloned into an AAV packaging construct under a CMV promoter together with a gRNA molecule targeting ELANE g35, CXCR4 or serpinA s12 under U6 promoter regulation between the ITR components (Table 4). AAV particles were produced by co-transfection of all packaging component plasmids into HEK293 cells, and particles purification (VectorBuilder).

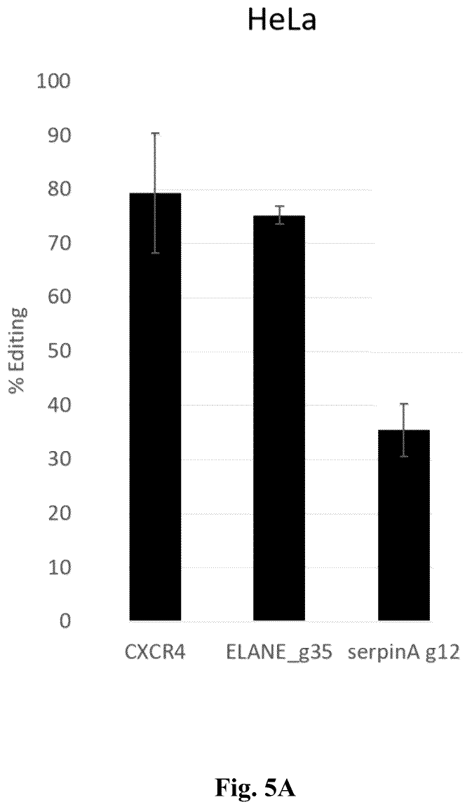

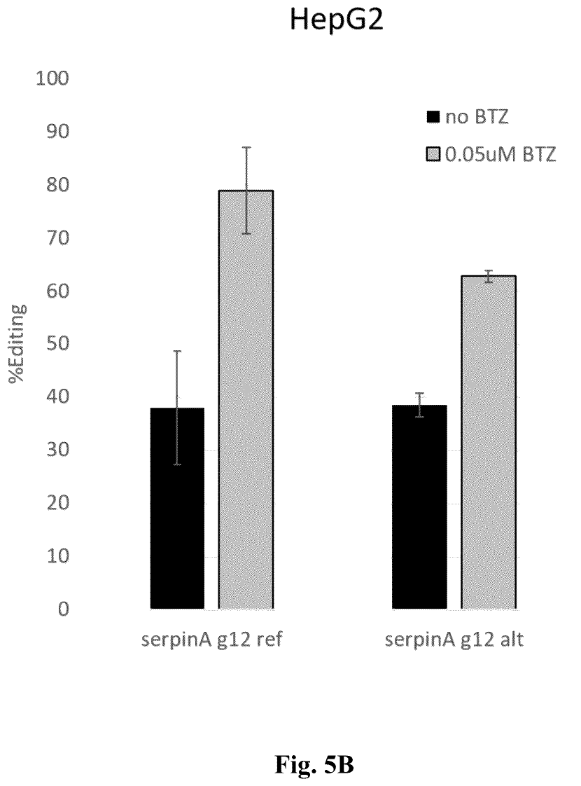

AAV particles containing OMNI-79 and a gRNA molecule were used to infect HeLa cells at MOI of 100,000 particles/cell in a 48-well plate format. At 72 hours cells were lysed, and their genomic DNA content was used in a PCR reaction which amplified the corresponding putative genomic targets. Amplicons were subjected to NGS and the resulting sequences were then used calculate the percentage of editing events. As can be seen in FIG. 5A, editing was observed in all sites tested. Editing level using AAV delivery was higher in all three (3) sites, compared with DNA transfection (see table 5).

We next tested editing efficiency of OMNI-79 by AAV delivery in the difficult to transfect cell line HepG2. AAV particles were used to infect HepG2 cells at MOI of 100,000 particles/cell in a 48-well plate format with or without a reversible proteasome inhibitor Bortezomib (BTZ). Since serpinA s12 was designed to target a SNP site (rs6647) both gRNA molecules targeting the ref or SNP sequences were tested. As can be seen in FIG. 5B, editing of serpinA s12 site in HepG2 is comparable to the editing observed in HeLa cells. Adding BTZ resulted in increased editing by 1.5-2 fold.

### Purifying OMNI-79 Protein

The OMNI-79 open-reading frame was cloned into bacterial expression plasmids (T7-NLS-OMNI-NLS-HA-His-tag, pET9a, Table 4) and expressed in KRX cells (Promega). Cells were grown in the following growth media Terrific Broth+0.4% glycerol+0.1% rhamanose+0.05% glucose. Culturing was done for four (4) hours until a OD600 nm reading of 5-6 was attained and the temperature was lowered to 18° C. 16-20 hours before harvesting and freezing cells at −80° C. Cell paste was resuspended in lysis buffer (20 mM Hepes, 1000 mM NaCl, 50 mM imidazole pH7.5, 1 mM TCEP) supplemented with EDTA-free complete protease inhibitor cocktail set III (Calbiochem). Cells were lysed using MC Multi Shot (Constant Systems) French press. Cell disruption was measured by the drop of OD600 nm to less than 10% of starting the OD600 nm. Clarification of the lysate was done in LYNX6000 centrifuge (Thermo Scientific) at 45,000×g for 30 minutes at 4° C. The cleared lysate was incubated with Ni Sepharose 6 Fast Flow resin (Cytiva). The resin was loaded onto gravity column and washed with wash buffer (20 mM Hepes, 500 mM NaCl, 50 mM imidazole pH7.5, 1 mM TCEP) and OMNI protein was eluted with elution buffer (20 mM Hepes, 200 mM NaCl, 500 mM imidazole, 1 mM TCEP). The elution sample was loaded using AKTA Avant (Cytiva) on pre-equilibrated HiTrap SP 5 ml (Cytiva) (20 mM Hepes pH7.5, 200 mM NaCl) and eluted using a linear gradient of 20 column volumes 0 to 100% buffer B (20 mM Hepes, 1000 mM NaCl pH7.5). Fractions of the OMNI-79 nuclease were pooled and concentrated and loaded onto a centricone (Amicon Ultra ultra 15 50K, Merck). The concentrated OMNI-79 protein was further purified by SEC on HiLoad 16/600 Superdex 200 pg-SEC, AKTA Pure (Cytiva) with a 20 mM Hepes pH 7.5, 300 mM NaCl, 10% glycerol. Fractions containing OMNI-79 protein were pooled and concentrated and loaded onto a centricone (Amicon Ultra 15 50K, Merck) with a final storage buffer of 20 mM Hepes pH 7.5, 300 mM NaCl, 10% glycerol, 1 mM TCEP. Purified OMNI protein was concentrated to 10-20 mg/ml, filtered with SpinX® (Merck), aliquoted, flash-frozen in liquid nitrogen, and stored at −80° C.

### OMNI-79 Spacer Optimization

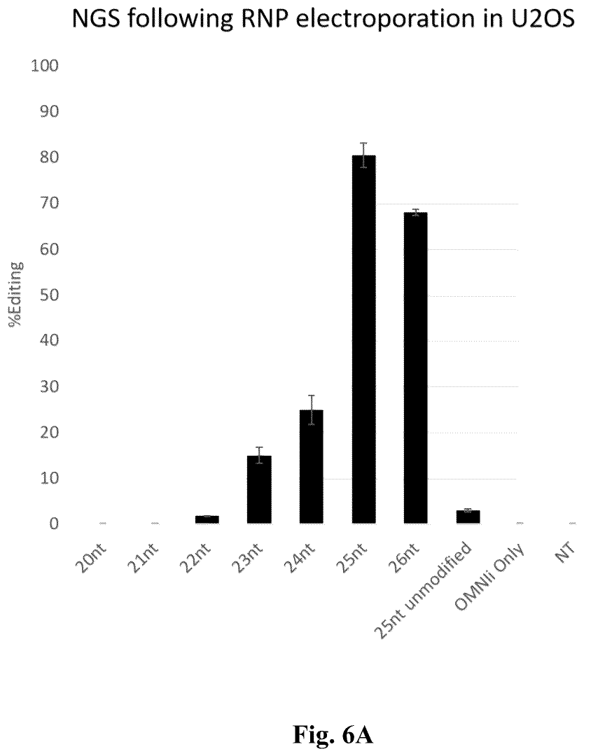

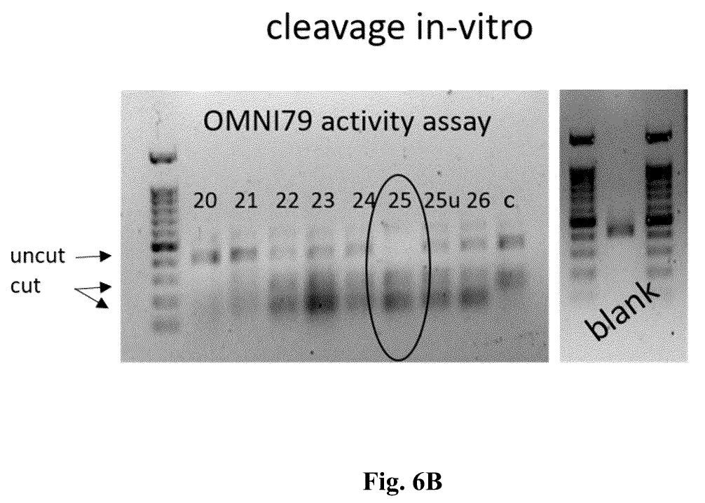

Synthetic sgRNA molecules of OMNI-79 were synthesized with three 2′-O-methyl 3′-phosphorothioate at the 3′ and 5′ ends (Agilent). An activity assay of OMNI-79 RNP with different spacer lengths (20-26 nucleotides) of guide 35 (FIG. 6B, Table 6). Briefly, 4 pmol of OMNI-79 nuclease were mixed with 6 pmol of synthetic guide. After 10 mins of incubation at room temperature, the RNP complexes were reacted with 100 ng of on-target template. Only spacers larger than 22 nucleotides show cleavage of the on-target template. Furthermore, full cleavage of the on-target template was observed with a 25-nucleotide spacer length only. Using a 25-nucleotide spacer, we compared gRNA molecules synthesized with or without the 2′-O-methyl 3′-phosphorothioate at the 3′ and 5′ ends, and found this modification to be important to achieve activity, likely due to RNA protection effects.

Spacer length optimization was also tested in a mammalian cell context. RNPs were assembled by mixing 100 uM nuclease with 120 uM of synthetic guide molecules having different spacer lengths (20-26 nucleotides, Table 6) and 100 uM Cas9 electroporation enhancer (IDT). After 10 mins of incubation at room temperature, the RNP complexes were mixed with 200,000 pre-washed U2OS cells and electroporated using Lonza SE Cell Line 4D-Nucleofector™ X Kit with DN100 or program, according to the manufacture's protocol. At 72 hours cells were lysed, and their genomic DNA content was used in PCR reaction which amplified the corresponding putative genomic targets. Amplicons were subjected to NGS and the resulting sequences were then used calculate the percentage of editing events. As can be seen in FIG. 6A and Table 6, spacers of 20-23 nucleotides show a low editing level, the 24-nucleotide spacer shows a medium editing level, and spacers of 25-26 nucleotides show the highest editing level. In this context as well, 2′-O-methyl 3′-phosphorothioate at the 3′ and 5′ ends were found to be important for activity, likely due to RNA protection.

TABLE 1a
OMNI CRISPR nuclease sequences

| | SEQ ID | | SEQ ID NO | SEQ ID NO of DNA |
|---|---|---|---|---|
| | NO of | | of DNA | sequence codon |
| | Amino | | sequence | optimized for |
| “OMNI” | Acid | | encoding | encoding OMNI in |
| Name | Sequence | Source Organism | OMNI | human cells |
| OMNI-59 | 1 | Comamonadaceae | 9 | 17 |
| | | bacterium NML00- | | |
| | | 0135 | | |
| OMNI-61 | 2 | Demequina | 10 | 18 |
| | | sediminicola | | |
| OMNI-67 | 3 | Fuerstia | 11 | 19 |
| | | marisgermanicae | | |
| OMNI-76 | 4 | Nitrosomonas sp. | 12 | 20 |
| | | Nm33 | | |
| OMNI-79 | 5 | Novosphingobium sp. | 13 | 21 |
| | | SYSU G00007 | | |
| OMNI-80 | 6 | Paracoccus | 14 | 22 |
| | | bengalensis | | |
| OMNI-81 | 7 | Parvibium lacunae | 15 | 23 |
| OMNI-82 | 8 | Pelagicola sp. | 16 | 24 |
| | | LXJ1103 | | |

TABLE 1b
OMNI CRISPR nuclease sequences

| | | | Dead nuclease |
|---|---|---|---|
| | Nickase having | Nickase having | having inactivated |
| “OMNI” | inactivated RuvC | inactivated HNH | RuvC and HNH |
| Name | domain | domain | domains |
| OMNI-59 | (D24 or E557 or | (E644* or H645 | (D24 or E557 or |
| | H785 or D788) | or N668) | H785 or D788) and |
| | | | (E644* or H645 or |
| | | | N668) |
| OMNI-61 | (D19 or E528 or | (D609* or H610 | (D19 or E528 or |
| | H750 or D753) | or N633) | H750 or D753) and |
| | | | (D609* or H610 or |
| | | | N633) |
| OMNI-67 | (D8 or E503 or | (E584* or H585 | (D8 or E503 or |
| | H729 or D732) | or N607) | H729 or D732) and |
| | | | (E584* or H585 or |
| | | | N607) |
| OMNI-76 | (D12 or E543 or | (E630* or H631 | (D12 or E543 or |
| | H770 or D773) | or N654) | H770 or D773) and |
| | | | (E630* or H631 or |
| | | | N654) |
| OMNI-79 | (D8 or E502 or | (D586* or H587 | (D8 or E502 or |
| | H735 or D738) | or N610) | H735 or D738) and |
| | | | (D586* or H587 or |
| | | | N610) |
| OMNI-80 | (D8 or E523 or | (D607* or H608 | (D8 or E523 or |
| | H757 or D760) | or N631) | H757 or D760) and |
| | | | (D607* or H608 or |
| | | | N631) |
| OMNI-81 | (D12 or E527 or | (E615* or H616 | (D12 or E527 or |
| | H756 or D759) | or N639) | H756 or D759) and |
| | | | (E615* or H616 or |
| | | | N639) |
| OMNI-82 | (D6 or E524 or | (D608* or H609 | (D6 or E524 or |
| | H756 or D759) | or N632) | H756 or D759) and |
| | | | (D608* or H609 or |
| | | | N632) |

Tables 1a-1b. OMNI nuclease sequences: Tables 1a-1b list the OMNI name, its corresponding nuclease protein sequence, its DNA sequence, its human optimized DNA sequence, alternative positions to be substituted to generate a nickase having an inactivated RUVC domain, alternative positions to be substituted to generate a nickase having an inactivated HNH domain, and alternative positions to be substituted to generate a catalytically dead nuclease having inactivated RUVC and HNH domains. Substitution to any other amino acid is permissible for each of the amino acid positions indicated in the columns listing nickase or dead nucleases, except if followed by an asterisk, which indicates that any substitution other than aspartic acid (D) to glutamic acid (E) or glutamic acid (E) to aspartic acid (D) results in inactivation.

OMNI Guide Sequences
Table 2a

| | | OMNI-59 | OMNI-61 | OMNI-67 |
|---|---|---|---|---|
| crRNA:trac | crRNA | GUUCCGGUCA | GUUGCGGCCAG | GCUGUGGUUC |
| rRNA | (Repeat) | (SEQ ID NO: 25) | AGCU (SEQ ID | GUCGGG (SEQ |
| duplex V1 | | | NO: 30) | ID NO: 40) |
| | Partial | Not listed | Not listed | GCUGUGGUUC |
| | crRNA 1 | | | GUCGG (SEQ ID |
| | | | | NO: 41) |
| | Partial | Not listed | GUUGCGGCCAG | GCUGUGGUUC |
| | crRNA 2 | | A (SEQ ID | GU (SEQ ID NO: |
| | | | NO: 31) | 42) |
| | Partial | Not listed | GUUGCGGCCA | GCUGUGGUUC |
| | crRNA 3 | | (SEQ ID NO: 32) | (SEQ ID NO: 43) |
| | tracrRNA | UGGUCGCUAAC | GGCUCUGCCGCU | CCUGACUUAUC |
| | (Antirepeat) | (SEQ ID NO: 26) | AAC (SEQ ID NO: | ACAGU (SEQ ID |
| | | | 33) | NO: 44) |
| | Partial | Not listed | Not listed | CUGACUUAUC |
| | tracrRNA 1 | | | ACAGU (SEQ ID |
| | | | | NO: 45) |
| | Partial | Not listed | UCUGCCGCUAAC | ACUUAUCACA |
| | tracrRNA 2 | | (SEQ ID NO: 34) | GU (SEQ ID NO: |
| | | | | 46) |
| | Partial | Not listed | UGCCGCUAAC | UUAUCACAGU |
| | tracrRNA 3 | | (SEQ ID NO: 35) | (SEQ ID NO: 47) |
| TracrRNA | TracrRNA | AAGCUGAUGCU | AAGGAGAAACU | AAGGUUCUCU |
| sequences | Portion 1 | UUUGUAGCUAG | UGUUGGAUCAG | ACCGC (SEQ ID |
| | | AUGCAAAAAAU | GACUCCACAAGA | NO: 48) |
| | | G (SEQ ID NO: 27) | U (SEQ ID NO: 36) | |
| | tracrRNA | Not listed | AAGGAGAAACU | GGUUCUCUACC |
| | Portion 1- | | UGUU (SEQ ID | (SEQ ID NO: 49) |
| | partial | | NO: 37) | |
| | TracrRNA | GAAAGCCGGGC | GAGACGGCUCCC | ACGGCAAUGU |
| | Portion 2 | AUGCCCGGCUUU | UCGUGGGGCCG | GUUUACACAU |
| | | CGGCUUUU (SEQ | UUUU (SEQ ID | CCGUU (SEQ ID |
| | | ID NO: 28) | NO: 38) | NO: 50) |
| | TracrRNA | Not listed | Not listed | AAGGACGGUC |
| | Portion 3 | | | CUGGACCGUCC |
| | | | | UUUUUUU(SEQ |
| | | | | ID NO: 51) |
| sgRNA | sgRNA V1 | GUUCCGGUCAgaa | GUUGCGGCCAG | GCUGUGGUUC |
| Versions | | aUGGUCGCUAAC | AGCUgaaaGGCUC | GUCGGGgaaaCC |
| | | AAGCUGAUGCU | UGCCGCUAACAA | UGACUUAUCA |
| | | UUUGUAGCUAG | GGAGAAACUUG | CAGUAAGGUU |
| | | AUGCAAAAAAU | UUGGAUCAGGA | CUCUACCGCAC |
| | | GGAAAGCCGGG | CUCCACAAGAUG | GGCAAUGUGU |
| | | CAUGCCCGGCUU | AGACGGCUCCCU | UUACACAUCCG |
| | | UCGGCUUUU | CGUGGGGCCGU | UUAAGGACGG |
| | | (SEQ ID NO: 29) | UUU (SEQ ID NO: | UCCUGGACCGU |
| | | | 39) | CCUUUUUUU |
| | | | | (SEQ ID NO: 52) |

Table 2b

| | | OMNI-76 | OMNI-79 sgRNA 1 | OMNI-79 sgRNA 2 |
|---|---|---|---|---|
| crRNA:trac | crRNA | GUUCCGGCUAG | GUUGCCGCUGG | GUUGCCGCUG |
| rRNA | (Repeat) | AG (SEQ ID NO: | A (SEQ ID NO: 63) | GACCG (SEQ ID |
| duplex V1 | | 53) | | NO: 71) |
| | Partial | GUUCCGGCUAG | Not listed | GUUGCCGCUG |
| | crRNA 2 | A (SEQ ID NO: 54) | | GA (SEQ ID NO: |
| | | | | 72) |
| | Partial | GUUCCGGCUA | GUUGCCGCUG | GUUGCCGCUG |
| | crRNA 3 | (SEQ ID NO: 55) | (SEQ ID NO: 64) | (SEQ ID NO: 73) |
| | tracrRNA | CUCUGGACGCUA | UCCAGUUGUUA | CGGUCUGGCA |
| | (Antirepeat) | AC (SEQ ID NO: | AC (SEQ ID NO: | GUUAAC (SEQ |
| | | 56) | 65) | ID NO: 74) |
| | Partial | UCUGGACGCUA | Not listed | UCUGGCAGUU |
| | tracrRNA 2 | AC (SEQ ID NO: | | AAC (SEQ ID |
| | | 57) | | NO: 75) |
| | Partial | UGGACGCUAAC | CAGUUGUUAAC | UGGCAGUUAA |
| | tracrRNA 3 | (SEQ ID NO: 58) | (SEQ ID NO: 66) | C (SEQ ID NO: |
| | | | | 76) |
| TracrRNA | TracrRNA | AAGCUGAAAGA | AAGCAGCUUGA | AAGUGUCAGU |
| sequences | Portion 1 | UGCACCAAAUG | CUGCACCAAAU | ACGCAACAGA |
| | | AU (SEQ ID NO: | (SEQ ID NO: 67) | U (SEQ ID NO: |
| | | 59) | | 77) |
| | tracrRNA | GCUGAAAGAUG | GCAGCUUGACU | GUGUCAGUAC |
| | Portion 1- | C (SEQ ID NO: 60) | GC (SEQ ID NO: | GC (SEQ ID NO: |
| | partial | | 68) | 78) |
| | TracrRNA | AGGGUCGCUAU | AAGGCGGGGGC | AAGGGCGACG |
| | Portion 2 | AGGCGACCCUUU | UGCGGCCCUCGC | CUCCGGCGUCG |
| | | UU (SEQ ID NO: | UUUUUU (SEQ ID | CCUUUUUU |
| | | 61) | NO: 69) | (SEQ ID NO: 79) |
| sgRNA | sgRNA V1 | GUUCCGGCUAG | GUUGCCGCUGG | GUUGCCGCUG |
| Versions | | AGgaaaCUCUGGA | AgaaaUCCAGUUG | GACCGgaaaCGG |
| | | CGCUAACAAGCU | UUAACAAGCAG | UCUGGCAGUU |
| | | GAAAGAUGCAC | CUUGACUGCACC | AACAAGUGUC |
| | | CAAAUGAUAGG | AAAUAAGGCGG | AGUACGCAAC |
| | | GUCGCUAUAGG | GGGCUGCGGCCC | AGAUAAGGGC |
| | | CGACCCUUUUU | UCGCUUUUUU | GACGCUCCGGC |
| | | (SEQ ID NO: 62) | (SEQ ID NO: 70) | GUCGCCUUUU |
| | | | | UU (SEQ ID NO: |
| | | | | 80) |

Table 2c

| | | OMNI-79 sgRNA 3 | OMNI-80 |
|---|---|---|---|
| crRNA:trac | crRNA | GUUGCCGCUGG | GUUGCGGUUGG |
| rRNA | (Repeat) | AC (SEQ ID NO: | (SEQ ID NO: 91) |
| duplex V1 | | 81) | |
| | Partial | GUUGCCGCUGG | Not listed |
| | crRNA 2 | A (SEQ ID NO: 82) | |
| | Partial | GUUGCCGCUG | GUUGCGGUUG |
| | crRNA 3 | (SEQ ID NO: 83) | (SEQ ID NO: 92) |
| | tracrRNA | GUCUGGCGGUU | CUGGCUGUUAA |
| | (Antirepeat) | AAC (SEQ ID NO: | C (SEQ ID NO: 93) |
| | | 84) | |
| | Partial | UCUGGCGGUUA | Not listed |
| | tracrRNA 2 | AC (SEQ ID NO: | |
| | | 85) | |
| | Partial | UGGCGGUUAAC | UGGCUGUUAAC |
| | tracrRNA 3 | (SEQ ID NO: 86) | (SEQ ID NO: 94) |
| TracrRNA | TracrRNA | AAGCAGCCAGUC | AAGCAGCUUGA |
| sequences | Portion 1 | UGCACCAGAU | CUGCACCAAAU |
| | | (SEQ ID NO: 87) | (SEQ ID NO: 95) |
| | tracrRNA | GCAGCCAGUCUG | GCAGCUUGACU |
| | Portion 1- | C (SEQ ID NO: 88) | GC (SEQ ID NO: |
| | partial | | 96) |
| | TracrRNA | AAGGGCGGCGC | AAGGGCAGGGC |
| | Portion 2 | UCCGGCGCCGCC | UGCGGCCCUGCC |
| | | UUUUUU (SEQ ID | UUUU (SEQ ID |
| | | NO: 89) | NO: 97) |
| sgRNA | sgRNA V1 | GUUGCCGCUGG | GUUGCGGUUGGg |
| Versions | | ACgaaaGUCUGGC | aaaCUGGCUGUUA |
| | | GGUUAACAAGC | ACAAGCAGCUU |
| | | AGCCAGUCUGCA | GACUGCACCAAA |
| | | CCAGAUAAGGG | UAAGGGCAGGG |
| | | CGGCGCUCCGGC | CUGCGGCCCUGC |
| | | GCCGCCUUUUUU | CUUUU (SEQ ID |
| | | (SEQ ID NO: 90) | NO: 98) |

Table 2d

| | | OMNI-81 | OMNI-82 |
|---|---|---|---|
| crRNA:trac | crRNA | GUUCCGGCUAG | GUUGCGGCUGG |
| rRNA | (Repeat) | AG (SEQ ID NO: | ACCGC (SEQ ID |
| duplex V1 | | 99) | NO: 109) |
| | Partial | Not listed | GUUGCGGCUGG |
| | crRNA 1 | | ACCG (SEQ ID |
| | | | NO: 110) |
| | Partial | GUUCCGGCUAG | GUUGCGGCUGG |
| | crRNA 2 | A (SEQ ID NO: | A (SEQ ID NO: |
| | | 100) | 111) |
| | Partial | GUUCCGGCUA | GUUGCGGCUG |
| | crRNA 3 | (SEQ ID NO: 101) | (SEQ ID NO: 112) |
| | tracrRNA | CUCUAGACGCUA | GCGGUCGAGCU |
| | (Antirepeat) | AC (SEQ ID NO: | GUUAAC (SEQ ID |
| | | 102) | NO: 113) |
| | Partial | Not listed | CGGUCGAGCUG |
| | tracrRNA 1 | | UUAAC (SEQ ID |
| | | | NO: 114) |
| | Partial | UCUAGACGCUA | UCGAGCUGUUA |
| | tracrRNA 2 | AC (SEQ ID NO: | AC (SEQ ID NO: |
| | | 103) | 115) |
| | Partial | UAGACGCUAAC | GAGCUGUUAAC |
| | tracrRNA 3 | (SEQ ID NO: 104) | (SEQ ID NO: 116) |
| TracrRNA | TracrRNA | AAGCUGAAAGA | AAGCAUUCGAU |
| sequences | Portion 1 | UGCACCAAAUG | UGCACCACAUU |
| | | (SEQ ID NO: 105) | (SEQ ID NO: 117) |
| | tracrRNA | GCUGAAAGAUG | GCAUUCGAUUG |
| | Portion 1- | C (SEQ ID NO: | C (SEQ ID NO: |
| | partial | 106) | 118) |
| | TracrRNA | GAAAGCCGCUA | GAAGCGCAGGG |
| | Portion 2 | UAUGCGGCUUU | CCACGGCCCUGC |
| | | CGUCUUUU (SEQ | GUUUU (SEQ ID |
| | | ID NO: 107) | NO: 119) |
| sgRNA | sgRNA V1 | GUUCCGGCUAG | GUUGCGGCUGG |
| Versions | | AGgaaaCUCUAGA | ACCGCgaaaGCGG |
| | | CGCUAACAAGCU | UCGAGCUGUUA |
| | | GAAAGAUGCAC | ACAAGCAUUCG |
| | | CAAAUGGAAAG | AUUGCACCACAU |
| | | CCGCUAUAUGCG | UGAAGCGCAGG |
| | | GCUUUCGUCUU | GCCACGGCCCUG |
| | | UU (SEQ ID NO: | CGUUUU (SEQ ID |
| | | 108) | NO: 120) |

TABLE 3
OMNI PAM Sequences
a

| | | OMNI-59 | OMNI-61 | OMNI-67 |
|---|---|---|---|---|
| TXTL | PAM General | NGCNNT | NSHNAC | NRRCM |
| Depletion | Activity | 0.95, 0.94 | 0.42, 0.54 | 0.95, 0.95 |
| | (1-Depletion | | | |
| | score)* | | | |
| | sgRNA | V1, V2 | V1, V3 | V1, V2 |
| Mammalian | PAM | No data shown | No data shown | No data shown |
| refinements | Mammlian | | | |

b

| | | OMNI-76 | OMNI-79 | OMNI-80 |
|---|---|---|---|---|
| TXTL | PAM General | NGSNNT | NGR | NNRGAY |
| Depletion | Activity | 0.2 | 0.97, 0.95 | 0.57, 0.48 |
| | (1-Depletion | | | |
| | score)* | | | |
| | sgRNA | V1 | V1, V2 | V1, V2 |
| Mammalian | PAM | No data shown | NGG | No data shown |
| refinements | Mammlian | | | |

c

| | | OMNI-81 | OMNI-82 |
|---|---|---|---|
| TXTL | PAM General | NRRAA | NRRNTT |
| Depletion | Activity | 0.92, 0.94 | 0.55, 0.58 |
| | (1-Depletion | | |
| | score)* | | |
| | sgRNA | V1, V2 | V2, V4 |
| Mammalian | PAM | No data shown | No data shown |
| refinements | Mammlian | | |

*Depletion score-Average of the ratios from two most depleted sites

TABLE 4
Plasmids and Constructs

| Plasmid/Viral | | | |
|---|---|---|---|
| Vector | Purpose | Elements | Example |
| pbNNC-3 | Expressing OMNI | T7 promoter HA Tag-Linker-OMNI | pbNNC3-OMNI-79 (SEQ ID |
| | polypeptide in the | ORF (Human optimized)-SV40 NLS- | NO: 121) |
| | bacterial system | 8XHisTag-T7 terminator | |
| pbSGR T1/T2 | Expressing OMNI | T7promoter-T1/T2 spacer sgRNA | pbSGR2-T2-OMNI-79 V1 |
| | sgRNA in the bacterial | scaffold-T7 terminator | (SEQ ID NO: 122) |
| | system | | |
| pbPOS T2 | Bacterial/TXTL | T2 protospacer-8N PAM library- | pbPOS T2 library (SEQ ID |
| library | depletion assay | chloramphenicol acetyltransferase | NO: 123) |
| pET9a | Expression and | T7 promoter-SV40 NLS-OMNI | pET9a-OMNI-79 (SEQ ID NO: |
| | purification of OMNI | ORF (human optimized)-HA-SV40 | 124) |
| | proteins | NLS-8 His-tag-T7 terminator | |
| pmOMNI | Expressing OMNI | CMV promoter-Kozak-SV40 NLS- | pmOMNI-79 (SEQ ID NO: |
| | polypeptide in the | OMNI ORF (human optimized)- | 125) |
| | mammalian system | HA-SV40 NLS-P2A-mCherry- | |
| | | bGH poly(A) signal | |
| pmGuide | Expressing OMNI | U6 promoter-Endogenic spacer | pmGuide T2- OMNI-79 V1 |
| T2/Endogenic | sgRNA in the | sgRNA scaffold | (SEQ ID NO: 126) |
| site | mammalian system | | |
| pPMLl3.1 | Viral vector for PAM | L TR-HIV-1 ∪ -CMV promoter- | pPMLl3.1 (SEQ ID NO: 127) |
| | library in mammalian | T2-PAM library (6N)-GFP- | |
| | cells | SV40 promoter-blastocydin S | |
| | | deaminase-LTR | |
| AAV viral | AAV viral vector | AAV-U6 promoter-spacer | AAV-OMNI-79 (SEQ ID NO: |
| vector | encoding OMNI | sequence (e.g. serpina s12, SEQ ID | 128) |
| | nuclease and guide | NO: 194)-scaffold sequence (e.g. | |
| | RNA molecule | DNA-encoding sequence of SEQ ID | |
| | | NO: 241)-CMV promoter-OMNI | |
| | | CRISPR nuclease sequence (e.g. SEQ | |
| | | ID NO: 21)-BGH terminator | |

TABLE 4
Appendix-Details of construct elements

| Element | Protein Sequence | DNA sequence |
|---|---|---|
| HA Tag | SEQ ID NO: 129 | SEQ ID NO: 133 |
| NLS | SEQ ID NO: 130 | SEQ ID NO: 134 |
| P2A | SEQ ID NO: 131 | SEQ ID NO: 135 |
| mCherry | SEQ ID NO: 132 | SEQ ID NO: 136 |

TABLE 5
Activity of OMNIs in human cells on endogenous genomic targets

| | | | | 3′ (PAM | | | % | % |
|---|---|---|---|---|---|---|---|---|
| | | Corresponding | | containing) | | | editing | transfection |
| | Genomic | Spacer | Spacer | genomic | % | % | in neg | in neg |
| Nuclease | site | name | sequence | sequence | indels | transfection | control | control |
| OMNI-59 | CXCR4 | OMNI59_CXCR4_S1 | SEQ ID | SEQ ID | 2.8 | 41.7 | 0.05 | 36 |
| | site 1 | | NO: 137 | NO: 159 | | | | |
| | CXCR4 | OMNI59_CXCR4_S2 | SEQ ID | SEQ ID | 0 | 44.8 | 0.05 | 36 |
| | site 2 | | NO: 138 | NO: 160 | | | | |
| | PDCD1 | OMNI59_PDCD1_S1 | SEQ ID | SEQ ID | 0 | 44.5 | 0.05 | 36 |
| | site 1 | | NO: 139 | NO: 161 | | | | |
| | PDCD1 | OMNI59_PDCD1_S2 | SEQ ID | SEQ ID | 0 | 53 | 0.05 | 36 |
| | site 2 | | NO: 140 | NO: 162 | | | | |
| | TRAC | OMNI59_TRAC_S1 | SEQ ID | SEQ ID | 0 | 44.5 | 0.05 | 36 |
| | site 1 | | NO: 141 | NO: 163 | | | | |
| | TRAC | OMNI59_TRAC_S2 | SEQ ID | SEQ ID | 0.3 | 42 | 0.05 | 36 |
| | site 2 | | NO: 142 | NO: 164 | | | | |
| OMNI-67 | CXCR4 | OMNI67_CXCR4_S3 | SEQ ID | SEQ ID | 0/0 | 87/69 | 0.00 | 86/61.5 |
| | site 1 | | NO: 143 | NO: 165 | | | | |
| | CXCR4 | OMNI67_CXCR4_S4 | SEQ ID | SEQ ID | 0/0 | 94/80 | 0.00 | 86/61.5 |
| | site 2 | | NO: 144 | NO: 166 | | | | |
| | PDCD1 | OMNI67_PDCD1_S3 | SEQ ID | SEQ ID | 0/0 | 94/81 | 0.00 | 86/61.5 |
| | site 1 | | NO: 145 | NO: 167 | | | | |
| | PDCD1 | OMNI67_PDCD1_S4 | SEQ ID | SEQ ID | 0/0 | 92/82 | 0.00 | 86/61.5 |
| | site 2 | | NO: 146 | NO: 168 | | | | |
| | TRAC | OMNI67_TRAC_S3 | SEQ ID | SEQ ID | 0/0 | 94/84 | 0.00 | 86/61.5 |
| | site 1 | | NO: 147 | NO: 169 | | | | |
| | TRAC | OMNI67_TRAC_S4 | SEQ ID | SEQ ID | 3.3/2.5 | 94/85 | 0.00 | 86/61.5 |
| | site 2 | | NO: 148 | NO: 170 | | | | |
| OMNI-79 | CXCR4 | OMNI79_CXCR4_S25 | SEQ ID | SEQ ID | 27/0.08 | 74/96 | 0/0 | 85/91.5 |
| | site 1 | | NO: 149 | NO: 171 | | | | |
| | CXCR4 | OMNI79_CXCR4_S26 | SEQ ID | SEQ ID | 0.42/0.07 | 42/83 | 0/0 | 85/91.5 |
| | site 2 | | NO: 150 | NO: 172 | | | | |
| | g58_Ref | OMNI79_g58_Ref | SEQ ID | SEQ ID | 0/0 | 58/89 | 0/0 | 85/91.5 |
| | | | NO: 151 | NO: 173 | | | | |
| | g35 | OMNI-79_g35 | SEQ ID | SEQ ID | 39.3/44 | 85/82.5 | 0/0 | 85/91.5 |
| | | | NO: 152 | NO: 174 | | | | |
| | PDCD1S24 | OMNI79_PDCD1_S24 | SEQ ID | TGGGCT | 6.702694334 | 92.80 | 0 | 91.3 |
| | | | NO: 181 | | | | | |
| | PDCD1S25 | OMNI79_PDCD1_S25 | SEQ ID | AGGATG | 0 | 91.13 | 0 | 91.3 |
| | | | NO: 182 | | | | | |
| | TRACS21 | OMNI79_TRAC_S21 | SEQ ID | TGGACT | 44.46183445 | 98.27 | 0 | 91.3 |
| | | | NO: 183 | | | | | |
| | TRACS24 | OMNI79_TRAC_S24 | SEQ ID | CGGAAC | 2.806238147 | 88.87 | 0 | 91.3 |
| | | | NO: 184 | | | | | |
| | EMXS2 | OMNI79_EMX_S2 | SEQ ID | GGGAGC | 44.95355661 | 82.47 | 0 | 91.3 |
| | | | NO: 185 | | | | | |
| | EMXS3 | OMNI79_EMX_S3 | SEQ ID | AGGGGACC | 1.48249255 | 70.70 | 0 | 91.3 |
| | | | NO: 186 | | | | | |
| | SAMD9 | OMNI79_SAMD9_rs070_1 | SEQ ID | SEQ ID | 0 | 51.8 | 0 | 41.97 |
| | rs070_1 | | NO: 187 | NO: 212 | | | | |
| | SAMD9 | OMNI79_SAMD9_rs070_2 | SEQ ID | SEQ ID | 0 | 51.57 | 0 | 41.97 |
| | rs070_2 | | NO: 188 | NO: 213 | | | | |
| | SAMD9 | OMNI79_SAMD9_rs201_alt | SEQ ID | SEQ ID | 1 | 56.43 | 0 | 41.97 |
| | rs20_1 | | NO: 189 | NO: 214 | | | | |
| | SAMD9 | OMNI79_SAMD9_rs499_1 | SEQ ID | SEQ ID | 28 | 55.3 | 0 | 41.97 |
| | rs499_1 | | NO: 190 | NO: 215 | | | | |
| | SAMD9 | OMNI79_SAMD9_rs499_2 | SEQ ID | SEQ ID | 0 | 48.67 | 0 | 41.97 |
| | rs499_2 | | NO: 191 | NO: 216 | | | | |
| | SAMD9L | OMNI79_SAMD9L_rs532_1 | SEQ ID | SEQ ID | 0 | 63.37 | 0 | 44.13 |
| | rs532_1 | | NO: 192 | NO: 217 | | | | |
| | SAMD9L | OMNI79_SAMD9L_rs532_2 | SEQ ID | SEQ ID | 29 | 65.17 | 0 | 44.13 |
| | rs532_2 | | NO: 193 | NO: 218 | | | | |
| | Serpina_S12 | OMNI79_Serpina_S12 | SEQ ID | SEQ ID | 33.37 | 75.17 | N/A | 80.13 |
| | | | NO: 194 | NO: 219 | | | | |
| | Serpina_S13 | OMNI-_79_Serpina_S13 | SEQ ID | SEQ ID | 8.55 | 86.2 | N/A | 80.13 |
| | | | NO: 195 | NO: 220 | | | | |
| | Serpina_S14 | OMNI79_Serpina_S14 | SEQ ID | SEQ ID | 7.86 | 84.37 | N/A | 80.13 |
| | | | NO: 196 | NO: 221 | | | | |
| | Serpina_S22 | OMNI79_Serpina_S22 | SEQ ID | SEQ ID | 19.14 | 68.37 | N/A | 80.13 |
| | | | NO: 197 | NO: 222 | | | | |
| | Serpina_S23 | OMNI79_Serpina_S23 | SEQ ID | SEQ ID | 55.57 | 87.6 | N/A | 80.13 |
| | | | NO: 198 | NO: 223 | | | | |
| | Serpina_S32 | OMNI79_Serpina_S32 | SEQ ID | SEQ ID | 5.38 | 71.03 | N/A | 80.13 |
| | | | NO: 199 | NO: 224 | | | | |
| | Serpina_S33 | OMNI79_Serpina_S33 | SEQ ID | SEQ ID | 2.49 | 76.77 | N/A | 80.13 |
| | | | NO: 200 | NO: 225 | | | | |
| | Serpina_S34 | OMNI79_Serpina_S34 | SEQ ID | SEQ ID | 8.47 | 63.07 | N/A | 80.13 |
| | | | NO: 201 | NO: 226 | | | | |
| | Serpina_S38 | OMNI79_Serpina_S38 | SEQ ID | SEQ ID | 5.89 | 26.23 | N/A | 80.13 |
| | | | NO: 202 | NO: 227 | | | | |
| | Serpina_S39 | OMNI79_Serpina_S39 | SEQ ID | SEQ ID | 6.95 | 91.43 | N/A | 80.13 |
| | | | NO: 203 | NO: 228 | | | | |
| | Serpina_S40 | OMNI79_Serpina_S40 | SEQ ID | SEQ ID | 6.63 | 91.27 | N/A | 80.13 |
| | | | NO: 204 | NO: 229 | | | | |
| | Serpina_S49 | OMNI79_Serpina_S49 | SEQ ID | SEQ ID | 16.97 | 89.1 | N/A | 80.13 |
| | | | NO: 205 | NO: 230 | | | | |
| | Serpina_S50 | OMNI79_Serpina_S50 | SEQ ID | SEQ ID | 13.15 | 64.5 | N/A | 80.13 |
| | | | NO: 206 | NO: 231 | | | | |
| | Serpina_S51 | OMNI79_Serpina_S51 | SEQ ID | SEQ ID | 13.22 | 79 | N/A | 80.13 |
| | | | NO: 207 | NO: 232 | | | | |
| OMNI-81 | CXCR4 | OMNI81_CXCR4_S6 | SEQ ID | SEQ ID | 30.21/13.2 | 92/72 | 0.04/0 | 94/70.7 |
| | site 1 | | NO: 153 | NO: 175 | | | | |
| | CXCR4 | OMNI81_CXCR4_S7 | SEQ ID | SEQ ID | 5.25/1.44 | 94/68 | 0/0 | 94/70.7 |
| | site 2 | | NO: 154 | NO: 176 | | | | |
| | PDCD1 | OMNI81_PDCD1_S7 | SEQ ID | SEQ ID | 47.84/7.94 | 95/41 | 0/0 | 94/70.7 |
| | site 1 | | NO: 155 | NO: 177 | | | | |
| | PDCD1 | OMNI81_PDCD1_S8 | SEQ ID | SEQ ID | 11.5/5.91 | 97/83 | 0/0 | 94/70.7 |
| | site 2 | | NO: 156 | NO: 178 | | | | |
| | TRAC | OMNI81_TRAC_S6 | SEQ ID | SEQ ID | 0/0 | 95/83 | 0.22/0.18 | 94/70.7 |
| | site 1 | | NO: 157 | NO: 179 | | | | |
| | TRAC | OMNI81_TRAC_S7 | SEQ ID | SEQ ID | 0.69 | 71 | 0.00 | 70.70 |
| | site 2 | | NO: 158 | NO: 180 | | | | |
| | EMXS11 | OMNI81_EMX_S11 | SEQ ID | CAGAA | 11.50 | 85.77 | 0 | 94.2 |
| | | | NO: 208 | | | | | |
| | EMXS12 | OMNI81_EMX_S12 | SEQ ID | AGGAA | 0.00 | 76.37 | 0 | 94.2 |
| | | | NO: 209 | | | | | |
| | B2MS8 | OMNI81_B2M_S8 | SEQ ID | AGGAA | 0.00 | 93.33 | 0 | 94.2 |
| | | | NO: 210 | | | | | |
| | B2MS9 | OMNI81_B2M_S9 | SEQ ID | TGAAAAA | 3.43 | 83.87 | 0.087625783 | 94.2 |
| | | | NO: 211 | | | | | |

Table 5. Nuclease activity in endogenous context in mammalian cells: OMNI nucleases were expressed in a mammalian cell system (HeLa) by DNA transfection together with an sgRNA expressing plasmid. Cell lysates were used for site specific genomic DNA amplification and NGS. The percentage of indels was measured and analyzed to determine the editing level. Each sgRNA is composed of the tracrRNA (see Tables 2a-2d) and the spacer detailed here. The spacer 3′ genomic sequence contains the expected PAM relevant for each OMNI nuclease. Transfection efficiency (% transfection) was measured by flow cytometry of the mCherry signal, as described above. All tests were performed in triplicates with two repeats. OMNI nuclease only (no guide) transfected cells served as a negative control. Results from repeat experiments are separated by a dash (“/”) symbol.

TABLE 6
OMNI-79 spacer length optimization

| Spacer Name | Spacer sequence |
|---|---|
| ELANE g35 19nt | GUCCGGGCUGGGAGCGGGU |
| | (SEQ ID NO: 233) |
| ELANE g35 20nt | AGUCCGGGCUGGGAGCGGGU |
| | (SEQ ID NO: 234) |
| ELANE g35 21nt | CAGUCCGGGCUGGGAGCGGGU |
| | (SEQ ID NO: 235) |
| ELANE g35 22nt | GCAGUCCGGGCUGGGAGCGGGU |
| | (SEQ ID NO: 236) |
| ELANE g35 23nt | UGCAGUCCGGGCUGGGAGCGGGU |
| | (SEQ ID NO: 237) |
| ELANE g35 24nt | CUGCAGUCCGGGCUGGGAGCGGGU |
| | (SEQ ID NO: 238) |
| ELANE g35 25nt | GCUGCAGUCCGGGCUGGGAGCGGGU |
| | (SEQ ID NO: 239) |
| ELANE g35 26nt | UGCUGCAGUCCGGGCUGGGAGCGGGU |
| | (SEQ ID NO: 240) |

Table 6. OMNI-79 spacer length optimization: OMNI-79 spacer optimization was performed using an RNA guide molecule comprising a version of the ELANE g35 spacer having different lengths. The OMNI-79 scaffold sequence used with each spacer is SEQ ID NO: 241. Results are shown in FIGS. 6A-6B.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
4. Behr (1994) “Gene transfer with synthetic cationic amphiphiles: Prospects for gene therapy”, Bioconjuage Chem 5:382-89.
5. Blaese et al. (1995) “Vectors in cancer therapy: how will they deliver”, Cancer Gene Ther. 2:291-97.
6. Blaese et al. (1995) “T lymphocyte-directed gene therapy for ADA-SCID: initial trial results after 4 years”, Science 270(5235):475-80.
7. Briner et al. (2014) “Guide RNA functional modules direct Cas9 activity and orthognality”, Molecular Cell 56:333-39.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Canver et al., (2015) “BCL11A enhancer dissection by Cas9-mediated in situ saturating mutagenesis”, Nature Vol. 527, Pgs. 192-214.
11. Chang and Wilson (1987) “Modification of DNA ends can decrease end-joining relative to homologous recombination in mammalian cells”, Proc. Natl. Acad. Sci. USA 84:4959-4963.
12. Charlesworth et al. (2019) “Identification of preexisting adaptive immunity to Cas9 proteins in humans”, Nature Medicine, 25(2), 249.
13. Chung et al. (2006) “Agrobacterium is not alone: gene transfer to plants by viruses and other bacteria”, Trends Plant Sci. 11(1):1-4.
14. Coelho et al. (2013) “Safety and efficacy of RNAi therapy for transthyretin amyloidosis” N. Engl. J. Med. 369, 819-829.
15. Crystal (1995) “Transfer of genes to humans: early lessons and obstacles to success”, Science 270(5235):404-10.
16. Dillon (1993) “Regulation gene expression in gene therapy” Trends in Biotechnology 11(5):167-173.
17. Dranoff et al. (1997) “A phase I study of vaccination with autologous, irradiated melanoma cells engineered to secrete human granulocyte macrophage colony stimulating factor”, Hum. Gene Ther. 8(1):111-23.
18. Dunbar et al. (1995) “Retrovirally marked CD34-enriched peripheral blood and bone marrow cells contribute to long-term engraftment after autologous transplantation”, Blood 85:3048-57.
19. Ellem et al. (1997) “A case report: immune responses and clinical course of the first human use of ganulocyte/macrophage-colony-stimulating-factor-transduced autologous melanoma cells for immunotherapy”, Cancer Immunol Immunother 44:10-20.
20. Gao and Huang (1995) “Cationic liposome-mediated gene transfer” Gene Ther. 2(10):710-22.
21. Haddada et al. (1995) “Gene Therapy Using Adenovirus Vectors”, in: The Molecular Repertoire of Adenoviruses 111: Biology and Pathogenesis, ed. Doerfler and Bohm, pp. 297-306.
22. Han et al. (1995) “Ligand-directed retro-viral targeting of human breast cancer cells”, Proc. Natl. Acad. Sci. USA 92(21):9747-51.
23. Humbert et al., (2019) “Therapeutically relevant engraftment of a CRISPR-Cas9-edited HSC-enriched population with HbF reactivation in nonhuman primates”, Sci. Trans. Med., Vol. 11, Pgs. 1-13.
24. Inaba et al. (1992) “Generation of large numbers of dendritic cells from mouse bone marrow cultures supplemented with granulocyte/macrophage colony-stimulating factor”, J Exp Med. 176(6):1693-702.
25. Jiang and Doudna (2017) “CRISPR-Cas9 Structures and Mechanisms”, Annual Review of Biophysics 46:505-29.
26. Jinek et al. (2012) “A programmable dual-RNA-guided DNA endonuclease in adaptive bacterial immunity”, Science 337(6096):816-21.
27. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
28. Judge et al. (2006) “Design of noninflammatory synthetic siRNA mediating potent gene silencing in vivo”, Mol Ther. 13(3):494-505.
29. Kohn et al. (1995) “Engraftment of gene-modified umbilical cord blood cells in neonates with adnosine deaminase deficiency”, Nature Medicine 1:1017-23.
30. Kremer and Perricaudet (1995) “Adenovirus and adeno-associated virus mediated gene transfer”, Br. Med. Bull. 51(1):31-44.
31. Macdiarmid et al. (2009) “Sequential treatment of drug-resistant tumors with targeted minicells containing siRNA or a cytotoxic drug”, Nat Biotehcnol. 27(7):643-51.
32. Malech et al. (1997) “Prolonged production of NADPH oxidase-corrected granulocytes after gene therapy of chronic granulomatous disease”, PNAS 94(22):12133-38.
33. Maxwell et al. (2018) “A detailed cell-free transcription-translation-based assay to decipher CRISPR protospacer adjacent motifs”, Methods 14348-57
34. Miller et al. (1991) “Construction and properties of retrovirus packaging cells based on gibbon ape leukemia virus”, J Virol. 65(5):2220-24.
35. Miller (1992) “Human gene therapy comes of age”, Nature 357:455-60.
36. Mir et al. (2019) “Type I-C CRISPR-Cas9 Biology, Mechanism and Application”, ACS Chem. Biol. 13(2):357-365.
37. Mitani and Caskey (1993) “Delivering therapeutic genes—matching approach and application”, Trends in Biotechnology 11(5):162-66.
38. Nabel and Feigner (1993) “Direct gene transfer for immunotherapy and immunization”, Trends in Biotechnology 11(5):211-15.
39. Nehls et al. (1996) “Two genetically separable steps in the differentiation of thymic epithelium” Science 272:886-889.
40. Nishimasu et al. “Crystal structure of Cas9 in complex with guide RNA and target DNA” (2014) Cell 156(5):935-49.
41. Nishimasu et al. (2015) “Crystal Structure of Staphylococcus aureus Cas9” Cell 162(5):1113-26.
42. Palermo et al. (2018) “Key role of the REC lobe during CRISPR-Cas9 activation by ‘sensing’, ‘regulating’, and ‘locking’ the catalytic HNH domain” Quarterly Reviews of Biophysics 51, e9, 1-11.
43. Remy et al. (1994) “Gene Transfer with a Series of Lipophilic DNA-Binding Molecules”, Bioconjugate Chem. 5(6):647-54.
44. Sentmanat et al. (2018) “A Survey of Validation Strategies for CRISPR-Cas9 Editing”, Scientific Reports 8:888, doi:10.1038/s41598-018-19441-8.
45. Sommerfelt et al. (1990) “Localization of the receptor gene for type D simian retroviruses on human chromosome 19”, J. Virol. 64(12):6214-20.
46. Van Brunt (1988) “Molecular framing: transgenic animals as bioactors” Biotechnology 6:1149-54.
47. Vigne et al. (1995) “Third-generation adenovectors for gene therapy”, Restorative Neurology and Neuroscience 8(1,2): 35-36.
48. Wagner et al. (2019) “High prevalence of Streptococcus pyogenes Cas9-reactive T cells within the adult human population” Nature Medicine, 25(2), 242
49. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
50. Yu et al. (1994) “Progress towards gene therapy for HIV infection”, Gene Ther. 1(1):13-26.
51. Zetsche et al. (2015) “Cpf1 is a single RNA-guided endonuclease of a class 2 CRIPSR-Cas system” Cell 163(3):759-71.
52. Zuris et al. (2015) “Cationic lipid-mediated delivery of proteins enables efficient protein based genome editing in vitro and in vivo” Nat Biotechnol. 33(1):73-80.

What is claimed is:

1. A non-naturally occurring composition comprising a CRISPR nuclease comprising a sequence having at least 90% identity to the amino acid sequence selected from the group consisting of SEQ ID NOs: 1, 2, 3, 4, 6, 7, or 8, or a nucleic acid molecule comprising a sequence encoding the CRISPR nuclease; wherein the CRISPR nuclease further comprises one or more nuclear localization sequences (NLSs).

2. The composition of claim 1, further comprising one or more RNA molecules, or a DNA polynucleotide encoding any one of the one or more RNA molecules, wherein the one or more RNA molecules and the CRISPR nuclease do not naturally occur together and the one or more RNA molecules are capable of forming a complex with the CRISPR nuclease and/or target the complex to a target site, and wherein the one or more RNA molecules are
a) a CRISPR RNA (crRNA) molecule and a transactivating CRISPR RNA (tracrRNA) molecule, or
b) a single-guide RNA (sgRNA) molecule.

3. The composition of claim 2, wherein the CRISPR nuclease comprises a sequence having at least 90% identity to the amino acid sequence set forth in SEQ ID NO: 1, and at least one RNA molecule comprises a sequence selected from the group consisting of SEQ ID NOs: 25-29.

4. The composition of claim 3, wherein the CRISPR nuclease comprises a sequence having at least 90% identity to the amino acid sequence set forth in SEQ ID NO: 1 and
a) at least one RNA molecule is a CRISPR RNA (crRNA) molecule comprising a guide sequence portion consisting of the sequence set forth in SEQ ID NO: 25; and further comprising a transactivating CRISPR RNA (tracrRNA) molecule comprising a sequence set forth in the group consisting of SEQ ID NOs: 26-28; or
b) at least one RNA molecule is a single-guide RNA (sgRNA) molecule comprising a guide sequence portion and a sequence selected from the group consisting of SEQ ID NOs: 25-29.

5. The composition of claim 1, wherein the CRISPR nuclease comprises a sequence having at least 90% identity to the amino acid sequence of SEQ ID NO: 1, and wherein the CRISPR nuclease is a nickase formed by an amino acid substitution at D24, E557, H785 or D788 of SEQ ID NO: 1, or
wherein the CRISPR nuclease comprises a sequence having at least 90% identity to the amino acid sequence of SEQ ID NO: 1, and wherein the CRISPR nuclease is a nickase formed by an amino acid substitution at E644, H645 or N668 of SEQ ID NO: 1, or
wherein the CRISPR nuclease is a catalytically inactive nuclease formed by an amino acid substitution at D24, E557, H785 or D788 of SEQ ID NO: 1, and an amino acid substitution at E644, H645 or N668 of SEQ ID NO: 1.

6. The composition of claim 2, wherein the CRISPR nuclease comprises a sequence having at least 90% identity to the amino acid sequence set forth in SEQ ID NO: 2, and at least one RNA molecule comprises a sequence selected from the group consisting of SEQ ID NOs: 30-39.

7. The composition of claim 6, wherein the CRISPR nuclease comprises a sequence having at least 90% identity to the amino acid sequence set forth in SEQ ID NO: 2 and
a) at least one RNA molecule is a CRISPR RNA (crRNA) molecule comprising a guide sequence portion and a sequence selected from the group consisting of SEQ ID NOs: 30-32; and further comprising a transactivating CRISPR RNA (tracrRNA) molecule comprising a sequence set forth in the group consisting of SEQ ID NOs: 33-38; or
b) at least one RNA molecule is a single-guide RNA (sgRNA) molecule comprising a guide sequence portion and a sequence selected from the group consisting of SEQ ID NOs: 30-39.

8. The composition of claim 1, wherein the CRISPR nuclease comprises a sequence having at least 90% identity to the amino acid sequence of SEQ ID NO: 2, and wherein the CRISPR nuclease is a nickase formed by an amino acid substitution at D19, E528, H750 or D753 of SEQ ID NO: 2, or
wherein the CRISPR nuclease is a nickase formed by an amino acid substitution at D609, H610 or N633 of SEQ ID NO: 2, or
wherein the CRISPR nuclease is a catalytically inactive nuclease formed by an amino acid substitution at D19, E528, H750 or D753 of SEQ ID NO: 2, and an amino acid substitution at D609, H610 or N633 of SEQ ID NO: 2.

9. The composition of claim 2, wherein the CRISPR nuclease comprises a sequence having at least 90% identity to the amino acid sequence set forth in SEQ ID NO: 3, and at least one RNA molecule comprises a sequence selected from the group consisting of SEQ ID NOs: 40-52.

10. The composition of claim 9, wherein the CRISPR nuclease comprises a sequence having at least 90% identity to the amino acid sequence set forth in SEQ ID NO: 3 and
a) at least one RNA molecule is a CRISPR RNA (crRNA) molecule comprising a guide sequence portion and a sequence selected from the group consisting of SEQ ID NOs: 40-43; and further comprising a transactivating CRISPR RNA (tracrRNA) molecule comprising a sequence set forth in the group consisting of SEQ ID NOs: 44-51; or
b) at least one RNA molecule is a single-guide RNA (sgRNA) molecule comprising a guide sequence portion and a sequence selected from the group consisting of SEQ ID NOs: 40-52.

11. The composition of claim 1, wherein the CRISPR nuclease comprises a sequence having at least 90% identity to the amino acid sequence of SEQ ID NO: 3, and wherein the CRISPR nuclease is a nickase formed by an amino acid substitution at D8, E503, H729 or D732 of SEQ ID NO: 3, or
wherein the CRISPR nuclease is a nickase formed by an amino acid substitution at E584, H585 or N607 of SEQ ID NO: 3, or
wherein the CRISPR nuclease is a catalytically inactive nuclease formed by an amino acid substitution at D8, E503, H729 or D732 of SEQ ID NO: 1, and an amino acid substitution at E584, H585 or N607 of SEQ ID NO: 3.

12. The composition of claim 2, wherein the CRISPR nuclease comprises a sequence having at least 90% identity to the amino acid sequence set forth in SEQ ID NO: 4, and at least one RNA molecule comprises a sequence selected from the group consisting of SEQ ID NOs: 53-62.

13. The composition of claim 12, wherein the CRISPR nuclease comprises a sequence having at least 90% identity to the amino acid sequence set forth in SEQ ID NO: 4 and
a) at least one RNA molecule is a CRISPR RNA (crRNA) molecule comprising a guide sequence portion and a sequence selected from the group consisting of SEQ ID NOs: 53-55; and further comprising a transactivating CRISPR RNA (tracrRNA) molecule comprising a sequence set forth in the group consisting of SEQ ID NOs: 56-61; or
b) at least one RNA molecule is a single-guide RNA (sgRNA) molecule comprising a guide sequence portion and a sequence selected from the group consisting of SEQ ID NOs: 53-62.

14. The composition of claim 1, wherein the CRISPR nuclease comprises a sequence having at least 90% identity to the amino acid sequence of SEQ ID NO: 4, and wherein the CRISPR nuclease is a nickase formed by an amino acid substitution at D12, E543, H770 or D773 of SEQ ID NO: 4, or
wherein the CRISPR nuclease is a nickase formed by an amino acid substitution at E630, H631 or N654 of SEQ ID NO: 4, or
wherein the CRISPR nuclease is a catalytically inactive nuclease formed by an amino acid substitution at D12, E543, H770 or D773 of SEQ ID NO: 4, and an amino acid substitution at E630, H631 or N654 of SEQ ID NO: 4.

15. The composition of claim 2, wherein the CRISPR nuclease comprises a sequence having at least 90% identity to the amino acid sequence set forth in SEQ ID NO: 6, and at least one RNA molecule comprises a sequence selected from the group consisting of SEQ ID NOs: 91-98.

16. The composition of claim 15, wherein the CRISPR nuclease comprises a sequence having at least 90% identity to the amino acid sequence set forth in SEQ ID NO: 6 and
a) at least one RNA molecule is a CRISPR RNA (crRNA) molecule comprising a guide sequence portion and a sequence selected from the group consisting of SEQ ID NOs: 91 and 92; and further comprising a transactivating CRISPR RNA (tracrRNA) molecule comprising a sequence set forth in the group consisting of SEQ ID NOs: 93-97; or
b) at least one RNA molecule is a single-guide RNA (sgRNA) molecule comprising a guide sequence portion and a sequence selected from the group consisting of SEQ ID NOs: 91-98.

17. The composition of claim 1, wherein the CRISPR nuclease comprises a sequence having at least 90% identity to the amino acid sequence of SEQ ID NO: 6, and wherein the CRISPR nuclease is a nickase formed by an amino acid substitution at D8, E523, H757 or D760 of SEQ ID NO: 6, or
wherein the CRISPR nuclease is a nickase formed by an amino acid substitution at D607, H608 or N631 of SEQ ID NO: 6, or
wherein the CRISPR nuclease is a catalytically inactive nuclease formed by an amino acid substitution at D8, E523, H757 or D760 of SEQ ID NO: 6, and an amino acid substitution at D607, H608 or N631 of SEQ ID NO: 6.

18. The composition of claim 2, wherein the CRISPR nuclease comprises a sequence having at least 90% identity to the amino acid sequence set forth in SEQ ID NO: 7, and at least one RNA molecule comprises a sequence selected from the group consisting of SEQ ID NOs: 99-108.

19. The composition of claim 18, wherein the CRISPR nuclease comprises a sequence having at least 90% identity to the amino acid sequence set forth in SEQ ID NO: 7 and
a) at least one RNA molecule is a CRISPR RNA (crRNA) molecule comprising a guide sequence portion and a sequence selected from the group consisting of SEQ ID NOs: 99-101; and further comprising a transactivating CRISPR RNA (tracrRNA) molecule comprising a sequence set forth in the group consisting of SEQ ID NOs: 102-107; or
b) at least one RNA molecule is a single-guide RNA (sgRNA) molecule comprising a guide sequence portion and a sequence selected from the group consisting of SEQ ID NOs: 99-108.

20. The composition of claim 1, wherein the CRISPR nuclease comprises a sequence having at least 90% identity to the amino acid sequence of SEQ ID NO: 7, and wherein the CRISPR nuclease is a nickase formed by an amino acid substitution at D12, E527, H756 or D759 of SEQ ID NO: 7, or
wherein the CRISPR nuclease is a nickase formed by an amino acid substitution at E615, H616 or N639 of SEQ ID NO: 7, or
wherein the CRISPR nuclease is a catalytically inactive nuclease formed by an amino acid substitution at D12, E527, H756 or D759 of SEQ ID NO: 7, and an amino acid substitution at E615, H616 or N639 of SEQ ID NO: 7.

21. The composition of claim 2, wherein the CRISPR nuclease comprises a sequence having at least 90% identity to the amino acid sequence set forth in SEQ ID NO: 8, and at least one RNA molecule comprises a sequence selected from the group consisting of SEQ ID NOs: 109-120.

22. The composition of claim 21, wherein the CRISPR nuclease comprises a sequence having at least 90% identity to the amino acid sequence set forth in SEQ ID NO: 8 and
a) at least one RNA molecule is a CRISPR RNA (crRNA) molecule comprising a guide sequence portion and a sequence selected from the group consisting of SEQ ID NOs: 109-112; and further comprising a transactivating CRISPR RNA (tracrRNA) molecule comprising a sequence set forth in the group consisting of SEQ ID NOs: 113-119; or
b) at least one RNA molecule is a single-guide RNA (sgRNA) molecule comprising a guide sequence portion and a sequence selected from the group consisting of SEQ ID NOs: 109-120.

23. The composition of claim 1, wherein the CRISPR nuclease comprises a sequence having at least 90% identity to the amino acid sequence of SEQ ID NO: 8, and wherein the CRISPR nuclease is a nickase formed by an amino acid substitution at D6, E524, H756 or D759 of SEQ ID NO: 8, or
wherein the CRISPR nuclease is a nickase formed by an amino acid substitution at D608, H609 or N632 of SEQ ID NO: 8, or
wherein the CRISPR nuclease is a catalytically inactive nuclease formed by an amino acid substitution at D6, E524, H756 or D759 of SEQ ID NO: 8, and an amino acid substitution at D608, H609 or N632 of SEQ ID NO: 8.

24. The composition of claim 2, wherein the composition is an engineered, non-naturally occurring composition comprising a CRISPR associated system comprising:
one or more RNA molecules, or one or more nucleotide sequences encoding the one or more RNA molecules, wherein at least one of the one or more RNA molecules comprising a guide sequence portion linked to a direct repeat sequence, wherein the guide sequence portion is capable of hybridizing with a target sequence; and
a CRISPR nuclease comprising an amino acid sequence having at least 90% identity to the amino acid sequence selected from the group consisting of SEQ ID NOs: 1, 2, 3, 4, 6, 7, or 8, or a nucleic acid molecule comprising a sequence encoding the CRISPR nuclease; and
wherein the one or more RNA molecules hybridize to the target sequence, wherein the target sequence is adjacent to the 3′ end of a complimentary sequence of a Protospacer Adjacent Motif (PAM), and the one or more RNA molecules form a complex with the CRISPR nuclease.

25. A method of modifying a nucleotide sequence at a target site in a cell-free system or the genome of a cell comprising introducing into the cell-free system or the cell the composition of claim 2.

26. The method of claim 25, wherein the eukaryotic cell is a mammalian cell.

27. The method of claim 26, wherein the CRISPR nuclease forms a complex with the at least one RNA molecule or RNA molecules, and effects a DNA break in a DNA strand adjacent to a protospacer adjacent motif (PAM) sequence, and/or effects a DNA break in a DNA strand adjacent to a sequence that is complementary to the PAM sequence.

28. The method of claim 27, wherein the CRISPR nuclease comprises a sequence having at least 90% identity to SEQ ID NO: 1 and the PAM sequence is NGCNNT.

29. The method of claim 27, wherein the CRISPR nuclease comprises a sequence having at least 90% identity to SEQ ID NO: 2 and the PAM sequence is NSHNAC.

30. The method of claim 29, wherein the CRISPR nuclease comprises a sequence having at least 90% identity to SEQ ID NO: 3 and the PAM sequence is NRRCM.

31. The method of claim 27, wherein the CRISPR nuclease comprises a sequence having at least 90% identity to SEQ ID NO: 4 and the PAM sequence is NGSNNT.

32. The method of claim 27, wherein the CRISPR nuclease comprises a sequence having at least 90% identity to SEQ ID NO: 6 and the PAM sequence is NNRGAY.

33. The method of claim 27, wherein the CRISPR nuclease comprises a sequence having at least 90% identity to SEQ ID NO: 7 and the PAM sequence is NRRAA.

34. The method of claim 27, wherein the CRISPR nuclease comprises a sequence having at least 90% identity to SEQ ID NO: 8 and the PAM sequence is NRRNTT.

35. The composition of claim 1, wherein the CRISPR nuclease comprises a sequence having at least 95% identity to the amino acid sequence selected from the group consisting of SEQ ID NOs: 1, 2, 3, 4, 6, 7, or 8, or a nucleic acid molecule comprising a sequence encoding the CRISPR nuclease.

36. The composition of claim 1, wherein the CRISPR nuclease is linked to a further protein.

37. The composition of claim 2, further comprising a donor template molecule.

38. The composition of claim 37, wherein the donor template molecule is a DNA molecule.

39. The method of claim 25, wherein the CRISPR nuclease and one or more of the DNA molecules are introduced to the cell as a ribonucleoprotein (RNP) complex.

40. The method of claim 25, wherein the nucleic acid molecule comprising a sequence encoding the CRISPR nuclease is an mRNA molecule.

41. The method of claim 25, wherein the nucleic acid molecule comprising a sequence encoding the CRISPR nuclease is a DNA molecule.

42. The composition of claim 1, wherein the CRISPR nuclease is a nickase or a catalytically inactive nuclease.


SEQUENCE LISTING (243 sequences)

SEQ ID NO: 1 - protein, 1049 aa, Comamonadaceae bacterium
  source 1..1049: note = NML00-0135
MNVWKGTGFVSRKRNIIRYRLALDLGSTSLGWAILRLNADNQPTAILKAGVRIFSDGRNPKDGSSLAVTRRAARAMRRRRDRLLKRKARMLDKLIAHGFFPQDEAARKALEVLNPYQLRAEGLQRALMPGEFARAMFHINQRRGFKSNRKTDKKDSDSGALKTAISQLRQQLQNENARTVGEWLWQRLQAGQGTRARYRETRIATDTGKGKIDKSYDLYIDRQMVADEFDALWAVQAAFNPVLFNEQARAELRDTLLHQRPLRPAKPGRCTLLPEEERAPLALPSTQRFRILQEVNHLRILHPDLREEALTLDRRNAIVALLENRGKVTFQAMRRTLNLGDAVQFNLEDAKRRELKGNATTAALSKKELFGAAWHDFDEALQDDIVLRLVTEESEAELVQWLIENTGVDEARATAIANTGLPEGYGSLSRKALARIVPALRAEVITYDKAVQAAGFAHHSDLRFSFEYDSADVEQVGERIDKTTGEILPVSAFKQLPYYGKALQRHVAFGSGNPQDPEEKRYGKIANPTVHIGLNQVRRVVNDLIRRYGRPTEIVVELARELKQSREQKLEAQRKQADNQKRNARIRAEIAPILGISEERVKHADIQKWILWEELSFDVADRRCPYSGVQISARMLLSDEVEIEHILPFSQTLDDSLNNKTVSMRQANRIKGNRTPWQARQDFEAQGWPYEGMQQRAERMPRAKRYRFAPDGYERWLGEDQGFLARALNDTRYLSRIARDYLTLVCPGGVRVIPGRMTALLRAKFGLNGVLSLSGEKNRDDHRHHAVDACVIGVTDQGLLQRFAEASAMARQQGLEKLVETMPLPWETYPAHVQRAVQNIWVSHRPDHGHEGGMMEETSYGISKDGRIKQRRKADGSQGREISNLIRISEPSQPERHGVDAEGQPLPYKGYVGGSNYCIEITRNDKGKWEGEVISTFRAYQIVRKHGVARLRHPEMAQNGKALVMRLMIDDCVRLELDGREETMRVVVIPRNGQVFMAPLHEANVDARNRDKNDPFSYISKMAGSFLKAKARHITISPIGELHDPGFKG

SEQ ID NO: 2 - protein, 1021 aa, Demequina sediminicola
MTGTDATAHSHTPYRLGLDVGTGSLGWAVVELDTDGNPVRIVRTGSRIYGSGRKPKDFSSLAADRRAARQMRKQRDRYLQRRTRLMHELVAAGLMPEAEVERQKLKDLNPYLLRARGVKEELTAHELGRALFHLQQRRGFKSNRKTDRKDDDRSAMKSAIASLQSDLGDDTLGTYMWKRIQNGESVRSRPRKVGSKNEYDFYVNRAMVEDEFNQLWDYQSQSHGDLLTDEARIRVHDAIFSQRPLKPVDPGRCTFETDQRRAPKALPSSQLFRIYQELNAIRVIDPFSSDQADRPLTRQERDAGASFLLGRVKATFPQLKKAMFGPTKLQLSLEYGERKNILGDVVGSELRKAQHIGPDWETYDLATQDLIVTILLEADTDDEVIERLQAESSLSLDQVHGALEAPLPDDYLRLSHRAIGKILPHLKDEWNEEGNAPVMYDAAVRAAGYQSHSEFHSGVLEDTLPYYGKVLKRYTQEVSGSSQAATNPDEWEFGKIANPTVHIGLNQIRTVVNSLIDRYGLPTQIHVEVARDLGQSAEGRREAASNRAKNERANEALNARLTELGQRTNFANRERLRLYDEISVLNHRCVLTGIPIEMSRLFTNDYQVDHILPFSRTLDDSRGNKILVHHTANQFKGARSPFEAYSETADWDHILQRASDAFGATSPKFKRFSADAMDRYSNGEQDFIARQLNDTSYLARVTREYLGSIVDPDRILATPGRLTSLLRHHWGLNGLLSDAAEKNRSDHRHHAIDALVVALSERVTLKAVTDANRRAGDQGIERLLVDLPQPWEGFADHARESVDRIVVSHKPDHNEKGQLHEETAYGVLEGPDKKGRFLTRKRITDPAKGVVGSWEQPKWRDVIPLYRRGEGPDSTLPYKAYIGGSNYCIEIVRTAKGKWAGEVVSTHTANTAEYRAFMAEPGAYRAQSYAGGDLVMRLIANDTIAIEVGDAGRQIMRLCQLETVGAMYFANVREGNVAARSRARDNDFSLLKKAASTLQPLKARRVFVDPIGRVFDPDFKE

SEQ ID NO: 3 - protein, 1046 aa, Fuerstia marisgermanicae
MNYILGLDLGSASLGWAVLECTEVDGSLQPTRIERTGVRIFEAGVEGDIEQGRDASRAAKRREARQPRRQNWRTQQRKRKLFRLLQQHGLLPASEKDDAISRKAVFDQLDKELTEKHITEGDHTAHQHLPYLLRMLASGAKVKPFELGRAIYSLAQRRGFLSNRKADTDEKEDGVVKASISELGGQIAGRTIAQTFVEDISPDHEDPGRQRIRQRYTAREMFHDEFNRIRKQQQPHFDLADNDWDTVYKTIFFQRPLKSQRHRIGRCEIDGGQRCLDALDVFQQFRIWHAVQNLRLADAYSLGRDGRLTLEEQQKIVDALQTQATMTWGKVVTLLGLKRGTKFTIQEWNTKGLTGHRTNSAMMHVFGDEWLDRPLEERDAITKEVVYFRKPSAMRKRGQEAWGLSEEQAALLPSTRLEEAHARHSAATLAIFVERMSRGEDYSTIRKDITGKDDSEPLDQLPPLSKAGLDITNPAVIRGLTELRKVVNELVRQYGKPIGIRIELSRSLKNSRDKRIKLHKDNEDRRKRREKAIEGILKQIPGRYSGNDIEKWLLAEECGWHCPYTGRPISPSTLLGSQPQFDIEHIFPRRYLDNSFSNKTLCYHEFNRNVKKNQTAFDACSGLDSWDEILQRVNNFDGPVAALKRKRFLTAAKEIPDGFTSKHLNDNRYNAVVAKKYVAMLYGGLSDADGSQRVFAVTGGHTALLRREWGLNSILSGTEEKTRDDHRHHAVDAVVIALTDPARIQALVNAAELAEKKASRRFYEAVQDPWPKFSSKVADSINEIVVSHRPTRTLPGALHAESIYSKPHIDKDGNTNHRIRKHITKLSATELKKDKIVDPAIRDLVKAKLKELGESNPAKAFAEEKNHPFLTAKDGRKIPIHKVRVFADKKPRAIAKNERQRYVASGKDSNFASMIYAVVDKDGHEIKWEHKVITRLEAHERKTRNRTVNGEKVLLPDPTDFNDDKKRVFKFALCKNDTVMLEGPDGDDVICRIQKISQAEIQLCPLATPSVQGKARSKWNQIQSIDNLRKWNLRTVLISPTGIEHR

SEQ ID NO: 4 - protein, 1036 aa, Nitrosomonas sp.
  source 1..1036: note = Nm33
MLHKMRYRLALDLGSTSLGWAMIRLDANQRPCAVIKAGVRIFSNGRNPKDGSSLAVTRREARAMRRRRDRLLKRKARMMRTLIEYGFFPAEEAQRKALETLNPYKLRADGLDKALTPAEFGRVLFHINQRRGFKSNRKTDKKDTDSGALKTAISKLREILKTENCRTVGEWLHKRNQAGQTVRARYRQDKTIKDDGKAKIDKYYDLYIDRAMIEHEFNELWRKQAEFNPALFSSAAYTDLKDVLLYQRPLKPVKPGRCTFMSDEERAPLALPSTQRFRMYQEVNNLRILREGLKEEPLTLQQRDDLIVVLERNNKRTFTQIKKLLGVGGAVQFNFEDPKREELKGNTTNAILGKKEHFGEAWIAFDEAKQDAIVMQLIKEENEAKLIQWLQDETGIEEERAEIIANVGLPEGYGSLGTKALARILPELRRDVVTYDKAVQAAGFEHHSKLNQNRGIPGITFKIESIDQDTGEIKEFHIHKELPYYGEYLQRHVGFGSGKPEDPIEKRYGKIANPTVHIGLNQVRLAVNALIKRYGHPSEVIVEVARDLKQSKEQRSEENKRQAENQQRNNRLRTEIARILQINEEGIRRDDIEKMILWIELSADVADRKCPYSGVPISATMLLSDEVEIEHILPFSQTLDDSLNNKTVALRKANRVKGDRTPWEAQQDFAAQGWSYADILARAENMRKEKRYRFAEDGYKRWLKDDAGFLPRALNDTRYLSRVAREYLHLICPNTRVIPGRITAMLRSQFGLNKVLGLNGEKNRNDHRHHAVDACVIGVTDQGLLQKFAKASASAREKQLNRLVDNMESPWKNYQEHVQRAIDAIWVSHKPDHSHEGAMHNDTAYGLRGNGKVSFHKMMDGKREYIEDNLKVIEIADTKAAERHGLLPNGKPKPYKGYKGDSNYCIEIVRNEKGRWEGEVISTFEAYQLVREQGAAQLRHPALGISGKPLVMRLMIDDTVRLDVDGQSCTMRIAKLSSNGQIFMADICEANVDARNRNKEDSFAYISKMAGSLQTAKARRVTISPIGELRDSGFKG

SEQ ID NO: 5 - protein, 1062 aa, Novosphingobium sp.
  source 1..1062: note = SYSU G00007
MEKRLGLDIGTNSIGWCLYEGDSILDIGVRIFSDGRDPKSGASLAVDRRNARAMRRRRDRYLGRRSALIKALKAHGLFPAEQDAAKALEREDPYSLRVRALDHRLDPHQIGRAIFHLNQRRGFRSNRKADRVLGDQESGLISTATRVLDEAMAKSGARTLGEFLASRDTRRVRMRPDVKGYDFYPNRQHYLEEFEKIWDAQSQYHPDLLSQQAKSAIHRIIFHQRPLKPQAVGTCTFAGLHGIPGDETRLPKAHPLFQQRRLYEEVNQLEIVCASAPARKLTRDERDALILKLQDKKKVTFSTLARTIRLKEGERFNKESENRKDLAGDEVRAEMSDKTRFGRRWFHLSLDEQWSVIDRLLNEESTEDLLAWLEKEWSLPSDVAEAVANAHLPDGHGRFGLTATVRLLEHLKADVVTYAEAARRAGFHHSDFRDGACYDELPYYGEILSREIAPGKDEYGDPLERQWGKITNPTVHIGLNQLRRLINALVRRHGRPDFIFVELARELKLNEKQKADHKRRIKQTTDAARARAEKLREIGQRDSGSNRMLLRIWEELNPSNPLDRRCPYCAEPISIEMLMSGSADIDHIVPYSRCLDDSAANKVVAHNHCNRQKGNRTPWEQWGQTTRWPLIQEQVARMHRSKQWRFGPDAMERVDRDGGFIARQLTDTQYLSRIAAQYLSALYTPDEGRRVYAVTGRLTAMLRRLWGLNDILPDHNWVLNPHSNAPKNRLDHRHHAIDAAVVGATTPAMIQQVARAAARAEEQDLDRLFADLPPPWPGFREELQGRIMAAVVSHKPDHGRKGRPLPGRDSTSGRLHNDTAYGFTGRRNAKGMPIVVTRKPLLALKPEDLTDPERIPDPALQGALFEATRGATGKDFEKALRDFSRRDGPYQGIRRIRLTEALNVIPIRDRTGHAYKGVKGDANARFDVWRLPDGKWITRWKDRDGIEHSGIVSLFDAHQPSQVYHRPHPAAKKVLSLRQNDLVAVEHDGDPGKIMRVVKFSANGSITFAPHNEAGPLKTRDTDPADPFRYVTTVASGLKKMRARQVRIDELGKVHDPGPRED

SEQ ID NO: 6 - protein, 1075 aa, Paracoccus bengalensis
MTTTLGIDLGTSSLGWCLIEDEHRILDLGVIIFSAAAGAGRDPQSGAPLAEARREARSARRRRDRFIGRRSALLDKLIALGLLPGDPPAGHGRRRNQALPNAETKALADTDPHVLRRRALSEPLSPHEIGRAIFHLNTRRGFKSNRKADRGRNEPETGKIATAGQALDAALGKRTLGQFLADRIDAGQPARVRMGGENQAYDFYPQRSHLEAEFAAIWEAQEHHHPELLTDTARTAIHRILFFQRPLKTPEVGFCTFAGMSGVPHDERRLPKAHPLFQERRLYEEVNNLKVVAAGAAARDLTLDERDRLILKLRDNKKVTFATLAKKVLKLAEGERFNKESEARKDLAGDEVRAEMADKKRFGNRWTHFPLERQLQIIDRVQNEENPDILLAWLQSDCGLDKAAAVAVARTNLPEGHGRFGETATRRLIAALKAEVVTYDKAALAAGFHHSDHRTGEVYDLLPYYGEVLTREIAPGKAEYGDPLERQYGKVTNPTVHIGLRQLQKLVNAVIARHGRPDRIVIELARELKLNDKQKDEHQRRIRRDTEAAIRRGEKLVEAGIADTGANRALMRQWEELNPSNPLDRRCPYCGEPIGMAQIFNSLADIDHIIPYSRSLDDSPANKVLVHRNCNRQKGNKTPWDRWHEDEAKWEIISAQVARMHPSKQWRFGPDAMERLERDGGFAARQLTDTQYLARIADKYLRGLYPTADEGRVDVIPGRMTAMLRRVWGLNSLLPDHNFVENEHSSAPKNRLDHRHHAIDATVAAVTSLSRMQQIAAAAARSEEKELERLFDDLPHPWDGFREDLGACLARTVATHKPDHGRSAKPSRHRDVTAGKLHNDTAYGLTGLKTTDGKTPIVVHRVLLASLKPTQIADPDCIPDETLRNALWLATRDCSGKAFDQALARFAKEHPVFKGIRRVRIREPLNVIPIHDREGKPYKSYAGASNDRYNVWRMPDASWRHDVVSTFNAHRSDYRDLRPHPAAKKVLSLRQNDMIAVERNGGLREIMRVVKFNQAGRLTLCPPNEGGKLQNRDAAPNDADPFKYTYLSPSSLKNAKARQVRIDPLGRVFDPGPRE

SEQ ID NO: 7 - protein, 1045 aa, Parvibium lacunae
MKNKMQYRLALDLGTTSLGWAMLRVKPNPEGRLEPFAVVKAGVRIFSDGRNPKDGSSLAVTRREARAMRRRRDRLLKRKARMLQQLTAFGFFPTDLAERKALETLNPYELRAKGLDEPLSPYEFGRSLFHINQRRGFKSNRKTDKKENDSSALKAAIRRVASEIDGNQARTVGEWLYKRMLNGQPVRGRYRETKVQKEDGKTKIDKSYDLYIDRAMVEAEFEALWAKQASLNPAVYSEQAKATLKDVLLFQRNLRPVKPGRCTLIPTEERAPLALPSTQRFRIYQEVNNLRILREGLKDEALTLVQRDALVTALEQNNKRTFAQIKKLLGLDGQTQFNFEDPKRQELKGNTTSAILSHPKHFGDAWFGFDEAKQDGIVCQLLNEENESALIRWLMDHTGVDEAHAEAIANAALPEGYGSLSRAALAKILPELRKAVITYDKAAQAAGFDHHSHISPSTTGEILPELPYYGEALQRHVGFGTGNPDDVPEKRYGKIANPTVHIGLNQVRKVVNALIKRYGHPSEVIVEVARDLKQSKKQRDEENKRQAENQKRNERIRQDIAAMRPDRSEERVTRTDIQKWILWEELSFDPANRCCPYSGVQISAEMLMSDAVEIEHILPFSRTLDDSLNNKTVSMRQANRIKGNQTPFEAFGKDNALGVNYSDILMRAQQMPKAKRKRFAENALEEWLQNEKNFLARALNDTRYLSRVAREYVSLICPQATRVIPGQMTAQLRAKFGLNDILGLDGEKNRNDHRHHAVDACVIGVTDQGLLQRFAYASASARANGLARLVDTMPDPWPSYRQHVQRAVQNIYVSHKPDHSHEGAMFDETIYSATGKSRSAAKDRTVIPFIAKNWSHPDDHNKQRPFKGLITDVSQRHQNKPYKGLLSNSNYCIEIYSDEAGWGGHVLKTFDAYQIVRAHKNASEGMQALRNKHSSQNGHPLVMRLMIGDYIRAEIDGFLLLLQVLKINSSGSITFIKPNETNISARYLAKLAAQKAQKEGKPFDDIALNDVFFQKAISADSLRLFKARPVTLSPIGELRDPGFKG

SEQ ID NO: 8 - protein, 1071 aa, Pelagicola sp.
  source 1..1071: note = LXJ1103
MRLGLDIGTNSIGWWLYRTENDQITCVVDGGVRVFSDGRDPQSKESLAVDRRVARAQRRRRDRYLRRKAALMKRMAEAGLMPADPVQAKALQALDPYDLRARGLDEALPLAHFGRALFHLNQRRGFKSNRKADRGDNESGKIKDATARLDWAMRDARARTYGEFLHMRQDKADDPRRVPTVRTRLSVARRDNAEKEEAGYDFYPDRRHLSEEFDALWAAQAEHHTTLTDDLRDQIKTIIFHQRPLKAPEVGLCLFTDERRIPSAHPLNQRRILLETVNGLRIVARGEAARGLTREERDQIVHGLDNKGHTKTLSGMSMKLRAIGKVIKLRSDQSFTLETANRDAIACDPVRASLSHPERMGGVWTTLDEDAQWDVVQRLRAVQSDTEHEALVDWLMATHGLGQDYAQATANAPLPEGYGRLGLTATRKILAALEADVMSYSDAVAACGWSHSGGPTGEVLEALPYYGEILDRHVIPGTGVKTDEDVKRFGRITNPTVHIGLNQIRRLVNRIICVHGKPDQIVVEVARDLKNSEDQKREIQKTIRKNTDDAIKRGKKLVEELGQKDTGANRLILRLWENLGNDVMTRQCPYSGKRISAAMLFDGSCDVDHILPFSRTLDDSIWNKTLCLKEENRKKANKTPWEVWGETDQWDVIVANLKNLDRKQAWRFAPDAVERFEGENDFSARALKDTQYLSRVARAYLDALYDGADGKSHVWVVPGRLTEMLRRHWGLNGIEVLTDSDAQTVKSKNRQDHRHHAIDAAVVAATDRSLIQRISKIAKHDEQAGAEQVARSVPPPWDGFRDDVAGQIGRIIVSHRADHGRIDPTARAQGSDTTAGQLHMDTAYGIVDGGHVVSRKPLMSLGAGDIRKIRDPDLQRHLTRVTRGLDKKEFEQALASFAASRKLPDQSENPYFGLRRVRLLDALQDSARVPVRNATGNIYKAYKAGSNHCYEVWRMPDGKVKPWAISTFEAHQSGDGSKPHPAAKRLLRVFKRDMVVIERKGITVICYVQKMDVANGLFLVPHTEGNADARNRDKEDDFKFIQMSAASLIKARIRRVHVDEMGRMRDPGPPR

SEQ ID NO: 9 - DNA, 3147 bp, Comamonadaceae bacterium
  source 1..3147: mol_type = genomic DNA; note = NML00-0135
atgaatgtgtggaaagggactggtttcgtgtcaagaaaaagaaacatcattcgttaccgactggctttggatctgggctccacctcgctgggctgggccatcctgcgcctgaatgctgacaaccagcccaccgccatcctcaaggctggcgtgcgcatcttcagcgatggccgaaacccaaaggatggctcctctctggccgtgacccgccgcgccgcccgcgccatgcgccgccgtcgcgaccgcctgctcaagcgcaaggcgcgcatgctggacaagctgattgcacatggcttcttcccacaggacgaggccgcgcgcaaagcattggaggtgctcaatccctaccagttgcgcgccgagggcttgcagcgcgcactgatgccgggcgagttcgcccgcgcaatgtttcatatcaaccagcgccgcggcttcaagagcaaccgcaagaccgacaagaaagacagcgacagcggcgccctcaaaaccgccatcagccaactgcgccagcaactgcaaaacgaaaacgcccgcacggtaggcgaatggctgtggcaacgcctgcaagccggccaaggcacgcgtgcccgctaccgggaaacgcgcatcgccaccgacacaggcaagggcaagatcgacaaaagctacgacctctatatcgaccgccagatggtggccgatgagttcgatgcgctgtgggccgtgcaggcggccttcaaccccgtactgttcaacgagcaggcacgggccgaactgcgcgacaccctgctgcatcaacgcccgttgcgcccggccaagccagggcgctgcaccttgctgccggaagaagagcgcgccccgctggccctgcccagcacgcagcgttttcgcatcctgcaagaggtgaatcacctgcgcattctgcacccagacttgcgtgaagaagcattgacgctggatcggcgcaatgccatcgtggccttgctggagaacaggggaaaagtcacgttccaggccatgcgcagaacactcaacctgggtgatgccgtgcaattcaatttggaagacgccaagcgccgtgaactcaagggcaatgccaccacggcggcactgtcgaaaaaggaactgttcggggcagcgtggcacgactttgacgaagccttgcaggacgacatcgtgctgcgcctggtcacggaagaaagcgaagccgaactggtgcaatggctgatcgaaaacactggcgtggatgaggcacgcgccacggccattgccaacactggcctgccagaaggctacggcagcctcagccgcaaggcactggcgcgcatcgtgcctgcgctgcgcgccgaggtcatcacctacgacaaggcggtgcaggccgccggctttgcccatcacagcgatttgcgattcagtttcgagtacgacagcgccgatgtggagcaggtgggagagcgtatcgacaaaaccaccggcgaaatcctgcctgtgtctgccttcaagcagttgccctactacggcaaggcgctgcaacgtcacgtggcctttggcagtggcaacccgcaagacccggaagaaaaacgctacggcaagatcgccaaccccaccgtacatatcggcctgaaccaggtgcgccgcgtggtcaacgatttgatccgacgttatggccggccaaccgaaatcgtggtggaactggcgcgcgagctcaagcaaagccgtgagcaaaagctggaggcgcaacgcaagcaagccgacaaccaaaagcgcaatgcgcgcattcgcgcggaaattgcgcctatcttgggcatcagcgaagagcgggtgaagcatgcagacattcagaaatggatcctgtgggaggagttgagctttgacgtcgccgaccgccgctgcccctacagcggcgtgcagatcagcgcgcgcatgctgctcagcgatgaggtggaaatcgagcacatcctgcccttctctcaaacgctggatgacagcctgaacaacaaaaccgtttccatgcggcaagccaaccgcatcaaaggcaaccgcacgccttggcaggcgcggcaggactttgaggcgcagggctggccgtacgagggcatgcagcaacgcgccgaacgcatgccccgcgccaaacgctaccgcttcgcgcccgatggttacgaacgctggctgggtgaggaccaaggctttctggcgcgcgcgctgaatgacacgcgctatctctcgcgcatcgcccgggattacctcacccttgtctgccccggcggtgtgcgggtgattccggggcggatgacggctttgctgcgcgccaagttcggcctgaatggggtcttgagcctgagcggcgagaaaaaccgcgatgaccatcgtcaccatgccgtggacgcctgtgtcattggcgttactgaccagggtctgttgcagcgctttgccgaggccagcgcgatggctcgccagcaaggactggaaaagctggtggaaacgatgcccttgccttgggaaacctacccagcgcatgtgcagcgcgccgtacaaaacatctgggtcagccaccgccccgaccatgggcatgagggcggcatgatggaagagacttcctacggcatcagcaaggatgggcgcatcaagcagcggcgcaaggccgatggcagtcaagggcgtgagatcagcaatctgatccgcatcagcgaaccaagccagcccgagcgccacggcgtagacgccgaaggccagcctttgccctacaaaggctatgtcggcggcagcaactactgcatcgaaatcacgcgcaatgacaaaggcaaatgggagggcgaagtgatctcgaccttccgcgcctatcaaatcgtgcgcaaacatggcgtggcgcgcctgcgacatccagagatggcacagaacgggaaggcgctggtgatgcgcctgatgattgacgactgcgtgcgcctagaattggacggacgggaagagacgatgcgtgtggtggtaattcccaggaacggacaggtcttcatggcgccactgcacgaagccaatgtggatgcccgcaaccgggataagaatgatccgttctcctatatctcgaagatggccggctccttcctaaaagccaaagcccgccacatcaccatctcgcccatcggcgagctgcacgaccccggcttcaagggc

SEQ ID NO: 10 - DNA, 3063 bp, Demequina sediminicola
  source 1..3063: mol_type = genomic DNA
atgactggcactgacgccacagcacactcgcacacgccgtaccgcctcgggctcgatgtagggacaggatctttgggttgggccgtcgttgaactcgataccgacggcaatcctgtccgcatcgtgcgcaccggctcgcggatctatgggagcgggcgcaaacccaaggacttttcatcgttagcggccgaccggcgtgccgcacgtcagatgcgcaagcaacgcgaccgctatcttcaacgccgcacgcgcctcatgcatgaactggtggccgccggtctgatgcccgaggccgaggtggagcggcagaagctcaaggatctcaacccctacctgctacgcgctcgtggggttaaagaggaactgaccgcccacgaacttggcagggctctctttcatctccagcagcgccgtggcttcaagtcaaatcgcaagacagaccgcaaggacgacgaccgcagcgcgatgaagagcgccatcgcatcgctccaatccgacttgggcgacgacacccttggcacgtacatgtggaagcggatccagaacggcgaatctgtgcggtcccgtccgcgaaaggtcgggtcaaagaacgagtacgacttttacgtgaaccgcgcgatggtcgaggatgagttcaatcagttgtgggactaccaaagccagtcccacggcgacctcctcacggacgaagcccgcatccgcgttcatgacgccatctttagccaacgtccgctcaagccggtggacccggggcgctgcacgttcgaaacagatcagcgacgcgcaccgaaggcactcccgagctctcaactgttccgcatctaccaagaacttaacgcgatcagagtgatcgatccgttctcatcagaccaagcagatcgacccctgacgcgtcaggagcgcgatgccggcgcatcgttcctcctgggcagggtgaaggccaccttccctcaactcaagaaggcgatgtttgggcccacaaagctccaactctcacttgagtacggagaacgcaagaacatcctgggtgacgtcgtaggaagcgagttgcgcaaagcgcagcacatcggcccagactgggagacgtatgacctcgccacccaggacctcatcgtgacgatcctgctcgaggcagacactgacgatgaagtgatcgagcggcttcaggctgagtcgtcgctatcgctcgatcaggtccacggcgcgcttgaggcaccactcccggacgactacctacggctgagccatcgtgcgattggcaagatcctgcctcatctcaaggatgaatggaatgaagagggcaatgcaccggtgatgtatgacgcggccgtccgtgccgcgggataccaatcgcactcagagttccacagcggtgttcttgaagacacgctcccctattacgggaaggtgctgaagcgatacacgcaagaggtctccgggagcagccaagctgctacgaaccctgacgaatgggagttcggcaagatcgccaatccgaccgtgcacatcggcctcaatcagatccgcaccgtcgtgaactcactgattgaccgctacggcctgcccacacagattcatgtcgaggtagcccgcgacttgggccagtcggctgagggaaggcgtgaagcagcgtccaaccgtgccaagaatgaacgagcaaacgaagcgctcaacgcccgcctgaccgaactgggacaacgcaccaactttgccaatcgggagcggctgcgcctctacgatgagatctcagtcctcaatcacaggtgtgtcctgacaggcatccccatcgagatgtcgcgactgttcaccaatgactatcaggttgaccacatcctgccgttctcccggaccctggacgactcgcgcggaaacaagattctggttcaccacacggccaatcagttcaagggcgcccggagccccttcgaggcatactcggagacggcagactgggatcacatccttcaacgcgcaagcgatgcctttggcgctacgagccccaagttcaagcgcttttctgccgacgccatggacaggtactcgaacggcgagcaagacttcatcgcccgccaactcaacgacaccagctacctggcacgagtgacacgcgagtatctagggagcatcgtggatcccgaccggatcctagccacaccgggcagacttacttcgctcttgcgccaccactgggggctcaatggcctgctatccgatgccgcggagaagaaccgctcagatcaccggcaccacgccatcgatgcccttgtcgttgcactcagtgagcgagtaactctaaaggccgtcaccgacgccaaccgtcgtgccggtgaccaaggcatcgaacgcttgctagtcgacctcccccagccgtgggagggctttgcggatcatgcccgtgagtctgttgatcgcattgtggtgtctcacaagccagatcacaatgagaaaggccagctccacgaagagaccgcctatggagtcctggaagggcccgacaaaaagggcaggttcctcacccgcaagcgcatcacggaccctgccaaaggtgtggtaggcagttgggagcaacccaagtggcgtgacgtcattccgctgtacaggcgcggcgaggggccagactcgacccttccctacaaggcttacattggcggctccaactactgcatcgagatcgtccgaactgccaaggggaaatgggctggggaggtagtctccacgcacaccgccaacacggccgagtatcgcgctttcatggcagagccgggcgcgtatcgcgcccagagttacgccggtggtgacctcgtcatgcggctcatcgccaatgacaccatcgcaatcgaagtcggcgatgcgggtcgccagatcatgaggctgtgccagttggagacggtcggagctatgtacttcgcaaatgtgcgtgaaggcaacgtcgcagcacgttcacgggctcgagacaacgacttttcgttgctcaagaaggcggcaagcacacttcagccactcaaggctcggagagtctttgttgatcccattggcagggtttttgaccctgatttcaaggag

SEQ ID NO: 11 - DNA, 3138 bp, Fuerstia marisgermanicae
  source 1..3138: mol_type = genomic DNA
atgaactacattcttgggctggatcttggttccgcctcacttggctgggcagttttggaatgcaccgaagtcgacggcagcctgcagccgactcgcatcgaacgcacgggtgtgcgaattttcgaagccggcgtggaaggcgacatcgaacagggccgcgacgcctctcgagcagccaaacggcgagaagctcgtcaaccgcgtcgtcaaaactggcggacgcagcaacgaaaacgcaaactgttccgactattacagcaacacggactgctgccggcttcagaaaaggacgacgcgatcagccgcaaagccgtgttcgatcaacttgacaaggaactgaccgaaaaacacatcacggaaggcgatcacacagcccatcagcacctgccgtacctgctgcgaatgctggcatctggcgcaaaggtgaagccatttgaacttggacgagcgatctacagcctcgctcagcgccgtggctttctcagcaaccgcaaagctgacacagatgaaaaagaagacggcgttgtcaaagcgagcatctccgaacttggcggtcaaatagctggtcgcacgatcgctcagacgtttgtcgaagacatcagtccggaccatgaagatcctggtcgtcaacgcatacggcaacgctacacagcccgtgagatgtttcatgacgaattcaatcgaattcgaaaacagcagcaaccgcacttcgacctcgcggacaatgactgggataccgtttataaaacaatcttctttcagcgtccgttgaagtcgcagcgtcaccgcatcggccgttgcgaaatcgatggcggtcagcgttgtcttgatgctcttgatgtctttcagcagtttcgtatctggcacgcggtgcagaatctgcgactcgcggacgcctacagtttggggcgggacggtcggctgacgctggaagaacagcagaagattgtcgacgcattgcagactcaggccacgatgacgtggggaaaagtcgttacgctgctgggtctaaaacgcggcacgaagttcacaattcaggaatggaacacgaaagggctaacagggcatcgcaccaactctgcgatgatgcacgtcttcggcgatgaatggctggacaggccattggaagaacgagacgccatcacaaaggaagtcgtctacttccgcaaaccatcagcgatgagaaaacggggccaggaagcatggggactgtcagaggaacaggccgccctgctaccgtcaactcggctggaagaagctcacgcccgacattccgccgcgacattggccatctttgtcgaacgcatgagtcgtggcgaagactattccaccattcgcaaagacattaccggcaaagacgacagcgaaccactcgaccagcttccaccgctcagcaaagccgggctggacatcaccaaccccgccgtcattcgtggcctgacagaacttcgcaaggttgtgaacgaactggtgcggcaatacggcaagcccatcggcatccgcatcgaactgtctcggtcgctaaagaattcgcgagacaaacgcatcaagctgcacaaagataacgaagatcgtcgtaagcgtcgcgagaaggcgatcgaaggtattctgaagcaaatccctggccggtactcgggcaacgatatcgaaaaatggctactcgcagaagaatgcggctggcactgtccgtacaccggaagaccgatttcgccttccacactgcttgggtcgcaaccacaattcgacatcgagcacattttcccgcgccgatatctcgacaacagcttcagcaataagacgttgtgctatcacgaattcaatcgcaacgtcaaaaagaaccaaaccgccttcgatgcgtgttccggcctcgatagctgggacgaaatcctgcagcgtgtcaacaactttgatgggccggtcgcagcattaaaacgtaaacgctttctgaccgcagcgaaagaaattccggacggtttcacttcaaaacacctcaacgataatcgctacaacgcagttgtcgcgaagaaatacgtggccatgctgtatggcgggctgtcagacgcggatggtagtcagcgagtctttgctgtcacgggcggacacacggcactgctgcggcgagaatggggcctgaattcgattttaagcggaaccgaagagaaaacacgagacgaccatcggcaccacgccgtcgatgcggtggtgatcgcattgacagacccggcaagaattcaggctttggtaaacgctgcagagcttgccgaaaagaaagcgtctcgccgcttttacgaagccgtccaggacccgtggccgaagttcagcagcaaagtcgctgattcaatcaatgaaatagtggtttcccaccgtccgaccagaactcttccgggagctctgcatgcggaatcgatttatagcaaaccgcacatcgacaaagacggcaacacgaaccatcgcattcggaaacacatcaccaagctgtctgcaaccgaattgaagaaggacaagattgtcgacccagcgattcgcgatctggtgaaggccaaactgaaagaactcggcgaatccaatccggcgaaagccttcgccgaggagaagaaccacccgttcttaaccgcaaaagacggtcgcaaaataccgattcacaaagtgcgagtgtttgccgacaagaaacctcgggcaatcgccaaaaacgagcggcagcgctatgtcgccagcggaaaagattccaactttgcgtccatgatttacgccgtcgtcgacaaagacggccacgaaatcaaatgggaacacaaagtcatcacccgcctggaggcgcacgaacgtaagactcgtaatcgtacggtgaacggagagaaagtgctgctaccagatcccaccgacttcaacgacgacaagaagcgggtcttcaaattcgcgttgtgtaagaacgatacagtgatgctggaaggccccgacggagacgacgtgatttgtcggatacagaagatcagtcaagccgaaattcagctgtgtccactggcgacgccgtctgttcaaggaaaagctcgttcgaaatggaaccaaatccaatcaatcgacaatctgcgaaaatggaacctcagaacggttctaatttctccgacgggtattgagcatagg

SEQ ID NO: 12 - DNA, 3108 bp, Nitrosomonas sp.
  source 1..3108: mol_type = genomic DNA; note = Nm33
atgctgcataagatgcgctatcgactggcacttgatttaggatcaacttcactgggctgggcgatgattagattagatgccaatcagcgaccctgtgcggttatcaaagcgggggtgcgtattttttcgaacggccgcaatccgaaagatggttcttctttggctgtaacccgcagagaagcccgcgccatgcgtcgtcgtcgtgatcgtctgctcaaacgcaaggcacgcatgatgcgtacactcatagaatatggcttttttcctgccgaagaagcgcagcggaaggcactagaaacactcaatccttataaattacgtgctgatggattggataaagcactcactccggctgaatttggccgtgtattgtttcacatcaaccaacgacgcggattcaagagtaaccgcaaaactgataaaaaagatactgatagtggtgcgctaaaaaccgctatcagcaaattgcgcgaaatactgaaaacagaaaactgccgcaccgtgggtgaatggctgcacaaacgcaatcaagcaggtcaaactgttcgcgctcgctaccgccaagacaaaactatcaaggatgatggcaaggcaaaaattgacaagtattacgatctctatattgaccgcgccatgattgagcatgagtttaatgaattatggagaaagcaggcagaattcaatccagccctattcagcagtgcagcctacaccgacttgaaagacgtattgttgtatcaacgtccgcttaaacctgtcaaacctgggcgttgcacctttatgtccgatgaagaacgtgcaccgttagcattacctagcacccaacgcttccgtatgtatcaggaggttaataatctgcgtatcctgcgggaaggcttgaaggaagaacctctgacattgcagcaacgcgatgatctaattgttgtgctagaaagaaacaataaacgcacctttacgcaaattaagaaattgcttggcgttggcggtgcagttcagttcaactttgaagatccgaaacgtgaggaacttaaaggtaataccaccaacgccattcttggcaaaaaagaacattttggtgaggcttggattgcgttcgacgaagctaaacaagacgccattgtcatgcaattaatcaaagaggaaaacgaagctaaactcatccagtggctgcaagatgaaaccggcattgaggaagaacgtgctgaaatcattgccaatgttggattaccggaaggctatggcagtttaggtacaaaggcattagcacgaattctgccagaactgcgccgtgatgtggtgacatacgacaaggcggtacaagctgcaggcttcgagcaccacagtaaactcaatcaaaatagaggaattcccggcatcacgttcaaaatcgaaagcattgatcaagatactggtgagatcaaagaatttcacattcacaaggaattgccttattacggtgaatatctgcaacgccacgttggatttggcagcggcaaaccggaagatcccatcgaaaaacgatacggcaaaattgctaaccccaccgtgcatatcgggttaaatcaggtacgccttgcagtaaacgcgctcattaagcgctacggtcatcccagcgaagtcatagtcgaggtggcgcgtgacttgaaacaaagcaaagagcagcgcagcgaagaaaataagcggcaagctgaaaatcagcaacgcaacaatcggttacgcactgaaattgctagaattttacagattaatgaagagggcatccgtcgcgacgacattgagaagatgattttatggatcgaactcagtgctgatgtcgctgaccggaaatgcccttattcaggtgtaccgatcagcgcaaccatgctcctgagcgacgaagtggaaattgagcacattttgcccttctcacaaacgctcgatgacagtctcaacaacaaaacggtggcgctccgtaaagccaatcgcgttaaaggagaccggacaccgtgggaagcacagcaagattttgctgctcaaggctggagctatgcggatatcctcgccagggcagaaaacatgcgcaaagaaaaacgctatcgttttgccgaagatggttataaacgctggctgaaggatgatgcgggattcctaccgcgcgcactgaatgacacgcgctatttaagcagagtggcacgcgaatacctgcacctgatttgccccaatactcgtgtcatccctggtcgcataaccgccatgctgcgcagtcaatttggtctcaacaaggtgctcggcttgaacggagaaaagaaccgcaatgaccaccgccaccacgcggtggatgcctgcgtaattggcgtgactgatcagggtttgttgcaaaaattcgcaaaagccagtgccagcgcgcgtgaaaagcaactcaaccggctcgtggacaacatggagtcaccgtggaaaaactaccaagagcatgtacaacgcgccattgatgctatttgggttagccacaagccggatcacagccacgaaggcgcaatgcacaatgacacagcttatggcttgcgaggcaatggcaaagtcagtttccataaaatgatggatggtaagcgtgaatacattgaagataatctcaaagtcattgaaattgccgatacaaaagctgcagaacgccatggcttgttgcccaatggtaaacctaaaccctataaaggctacaaaggtgacagcaactactgcattgaaattgttcgcaatgaaaaaggcagatgggaaggagaggttatttctacctttgaggcttatcagctcgtgcgtgaacagggtgccgcacagcttcggcatccggctttgggtatcagcgggaaacctttggtaatgcggttaatgattgatgacacagttcgtttagatgttgacggtcaatcttgcacaatgcgaatagcgaaattaagtagtaatgggcaaatattcatggcagacatatgtgaagcaaacgttgatgcaagaaatcgaaataaagaagattcatttgcctacatttccaaaatggcaggttctcttcaaacagccaaagcacgccgcgtcaccatttctccgattggcgaactccgcgattccggtttcaagggg

SEQ ID NO: 13 - DNA, 3186 bp, Novosphingobium sp.
  source 1..3186: mol_type = genomic DNA; note = SYSU G00007
atggagaagcgcctgggactggatatcgggacaaactcgatcggttggtgcctgtatgaaggcgacagcattctcgatatcggcgtgagaatcttctcggacggacgagacccgaaatccggggcaagtcttgccgttgatcgccggaatgcgcgtgccatgcgtcggcgccgcgatcgttatcttggtcgtcgatccgctctcatcaaggcgctcaaagcccatggccttttccccgcagaacaggatgcggcaaaggcgctcgaacgcgaggatccatacagcttgagggttcgtgcgctcgatcatcggctggatccgcatcagatcggcagggcgatctttcacctgaaccagcgtcgtggtttccgctctaaccgcaaggccgacagggtattgggcgatcaggaaagcggactgatttccaccgccactcgtgttctggatgaggcgatggccaaaagcggcgcgcgcaccctcggcgagtttctcgcttcacgcgacacccgcagagtgcgcatgcggcctgacgtcaagggctatgatttctatcctaatcggcaacactatctcgaagagttcgagaagatctgggacgcgcaatcgcaataccaccccgatctgctgagccaacaggcgaaatccgccatccaccgaatcatctttcaccagcgcccgcttaagccgcaggctgtcggcacatgcactttcgccgggttacacggcatccctggcgatgaaacgcgcttgcccaaagcccatcccctgttccagcagcgcaggctttacgaggaagtcaaccagcttgagatcgtttgtgcaagcgcccctgcccgcaagctgacgcgcgacgaacgcgatgccctcatcctcaagctgcaggacaaaaagaaggtcactttctccaccctcgcgcgcaccataagattgaaggagggtgaacgcttcaacaaggaaagcgagaaccggaaggatctggcaggcgacgaagtgcgtgccgagatgagtgacaagacgcgctttggcaggcgatggtttcacctttcccttgatgagcagtggtctgttatcgaccgcttgctgaacgaggaaagcaccgaagacctgctggcctggctcgaaaaggaatggtcgctgcccagcgatgtcgccgaggcagtagccaatgcgcatctgccagacggccatggcaggtttggccttactgccaccgttcgcctgttggaacacctcaaggctgacgtggtgacttatgccgaagcagcaagacgtgcaggcttccaccacagcgactttcgcgatggcgcgtgctacgatgagcttccctactacggggagatactttcccgcgagatcgcgccaggcaaggacgaatacggcgatccgctggaacggcaatggggcaagattaccaatcccaccgttcacatcggactgaaccagttgcgacggctgatcaacgcgcttgtcagacggcacggtcgtcccgatttcattttcgtcgaactggcgcgtgagctgaagctgaacgaaaagcagaaggccgaccacaagcgccggatcaagcagacaaccgatgccgcccgcgccagggcagaaaagctgcgcgagatcggccagcgcgacagtggcagcaaccggatgctcctgcgcatatgggaagaactgaatccctccaatccacttgaccggcggtgcccctattgcgccgaacccatttcgatcgagatgctgatgagcggatcagccgatatcgatcacattgttccctattcgcgttgccttgatgacagcgccgcgaacaaggttgtcgcgcataaccactgcaatcggcagaaaggaaaccgcaccccatgggaacaatgggggcaaacgacgcgctggcccttgatccaggagcaggtcgctcgcatgcatcggtccaagcaatggcgctttggccccgatgcgatggaacgggtggaccgggatggtggcttcatcgctcgccagcttaccgacacccagtatctttcgcgcattgcggcgcagtatctttccgctctctatacacccgatgagggacggcgggtctatgccgtgactggccggttgacggcgatgctccgccgactgtggggactgaatgacatcctgcccgatcacaactgggttctgaacccgcacagcaacgcacccaagaaccggcttgatcaccgccaccatgccattgatgctgcggtggttggtgccacaactccggcaatgatccagcaggttgctagagcggcagcacgcgcggaagagcaggatcttgaccggctctttgccgatctgccaccgccatggccaggcttccgcgaggaactgcagggccgcatcatggccgctgttgtcagtcacaagccggatcatgggcgcaaggggcgtccccttccagggcgagacagcacctcaggccgcctgcacaacgatacggcctatggcttcaccggaaggcgcaatgccaagggcatgccgatcgtggtcacgcgaaagccgctgctggccctcaagcccgaagacctgaccgatccggaaagaatacccgacccggcactgcagggcgcgttgtttgaagccacgcggggcgcaaccggcaaggactttgaaaaggcgctgcgcgacttttcgcgaagggacggcccttatcagggcatccggcgcatacggttgaccgaagcgctcaacgtgattccgatccgtgatcgaacaggacacgcctataagggtgtaaagggcgatgccaatgcgcgtttcgatgtctggcggcttcctgatggcaaatggatcacgcggtggaaggaccgggacggcatcgaacattccggcatagtatcgctgttcgatgcacatcagccgagccaagtctatcaccgcccccaccctgccgcgaagaaggtgcttagcctgcggcagaatgatctcgttgcggttgaacacgatggcgacccaggcaagatcatgcgtgtggtcaagttttctgccaatggctcgatcaccttcgccccacataacgaagccgggccgctaaagacgcgtgataccgaccctgccgatccgttccgttatgtcaccactgttgcgagcggactgaagaagatgcgcgcgcggcaggtgcggatcgacgaactaggaaaagtccacgatccggggccgcgcgaagac

SEQ ID NO: 14 - DNA, 3225 bp, Paracoccus bengalensis
  source 1..3225: mol_type = genomic DNA
atgaccactacactcggcattgatcttggcacgtcgtcgttgggctggtgtctcatcgaagacgaacaccgcatcctcgatctcggcgtgatcatcttcagcgccgccgccggtgccgggcgcgacccgcaatcgggcgcacccctcgccgaagcgcggcgcgaggcccgttcggcgcggcggcggcgggacaggttcatcggtcgccgctcggctcttctggacaagctgatcgcgctcggcctgctgcccggagacccgcccgcgggccatgggcggcggcgcaaccaggcgctgccgaatgccgagacgaaggcgctggccgacaccgatccccatgtcctgcgccgccgggctttgtcggagccactttcgccgcacgagatcggccgggcgatcttccacctcaacacccggcgcggcttcaagtcgaaccgtaaggccgaccggggccgaaacgagcccgaaaccggcaagatcgccaccgccggccaggcgctggacgcggccttgggcaagcgcaccctcggccaatttctggccgaccggatcgacgccgggcaaccggcgcgggtgcggatgggtggcgagaaccaggcctatgatttctatccccagcgcagccatctcgaggcggagttcgcggcgatctgggaggcgcaggaacaccaccaccccgagttgctgaccgacacggcccggaccgcgatccatcgcatcctgttctttcagcgcccgctgaaaacgcccgaggtgggtttttgcaccttcgccggcatgagcggtgtgcctcacgacgagcgccggctgccaaaggcgcatccgctgttccaggaacggcggctctacgaggaggtgaacaacctcaaggtagtcgccgccggagccgctgcgcgcgacctgacgctggatgaacgggaccggctgatcctcaagttgcgcgacaacaagaaagtcaccttcgccactttggccaagaaggtgctgaaactggccgagggcgagcggttcaacaaggaaagcgaagcccgcaaggacctggcgggcgacgaggtccgggccgagatggcggacaagaagcgcttcggcaatcgctggacgcatttcccgctggagcggcagttgcagatcatcgaccgtgtgcagaacgaggaaaatcccgatatcctgctggcctggctgcaatccgactgcgggctggacaaggcggcggcggtcgcggtggccaggacgaacctgcccgaggggcacggccgcttcggcgagaccgcgacccggcggctgatcgcggctctaaaggccgaggtcgttacctatgacaaggccgcgctggcggcagggttccaccatagcgatcaccggacgggcgaggtttacgacctcctgccctattacggcgaggtcctgacccgtgagatcgcgcccggcaaggcggagtacggcgatccgctcgaacgccaatatggcaaggtcaccaatcccaccgtccatattggcctgcgccagttgcaaaagctggtgaatgcggtgatcgcccggcatggcaggcccgaccggatcgtcatcgaactcgcccgcgaactgaagctcaatgacaagcagaaggacgagcatcagcgacggatcaggcgggataccgaggcggcgatccgcaggggcgagaagcttgttgaggcgggtattgccgataccggcgccaaccgcgccctgatgcggcaatgggaggagctgaacccgtcgaaccccctcgaccgtcgctgcccctactgcggcgagccgattggcatggcgcagattttcaatagcctagcggatatcgaccatatcatcccctattccaggtcgctcgacgacagcccggcgaacaaagttttggtccatcgcaactgcaaccgccagaagggcaacaagacgccctgggatcggtggcacgaggacgaggcgaaatgggaaatcatctcggcccaggttgcgcggatgcacccttcgaaacaatggcgctttggccccgacgcgatggagcggctggaacgcgacggcggcttcgccgcccgccaactgactgatacccagtatctggcgcggatcgcggacaaatacctgcgcggcctttatccgactgctgatgagggccgggtcgatgtcatccccgggcgcatgaccgcgatgctgcgccgggtctggggcctgaacagcctgctgcccgaccataacttcgtcgagaacgaacattccagcgcaccgaagaaccggctggatcaccgccaccatgcgattgacgccacggtcgcggcggtgacatctctgtcccggatgcagcagatcgcagccgcagcggcccggtcggaggaaaaggaattggagcgcctgttcgacgatctgccgcatccttgggacggcttccgcgaggacctgggcgcgtgccttgcccgcacagtcgccacgcacaagcccgatcatggacgcagcgcgaaaccctcgcgccaccgcgatgtgacggcgggcaagctgcacaacgacaccgcctatggcctgaccggcctgaagaccaccgatggcaagacccctatcgtcgtgcaccgcgttctcctggcctcgctgaagcccacgcagatcgccgaccccgactgtattcccgacgaaaccctccgcaacgccctgtggctcgccacccgggactgttccggcaaggccttcgatcaggccctagcgcgcttcgccaaggagcatccggtgttcaagggcatccgccgggtgaggatacgcgagccgctgaacgtcatcccgatccacgacagggaaggaaaaccttacaaaagctatgccggggcgtccaacgaccgatacaatgtctggcggatgcccgacgcttcctggcggcatgatgttgtgtccaccttcaacgcgcaccggagcgattatcgcgaccttcgcccgcacccagcggcgaaaaaggttttatcgctacgccagaacgatatgatcgcggtggagcgtaatggtgggcttcgggagattatgcgggtcgttaagttcaaccaagccgggcggctgactctttgtccgccgaacgagggcggcaagcttcagaaccgggacgcggcaccgaacgacgccgatccgttcaaatacacttatctttctcccagctccctaaaaaacgccaaggcccgtcaggtccgcatcgatccactcggtcgcgtcttcgatcccggcccacgcgaa

SEQ ID NO: 15 - DNA, 3135 bp, Parvibium lacunae
  source 1..3135: mol_type = genomic DNA
atgaaaaacaaaatgcaataccgtttggcccttgatttaggcaccacctcacttggctgggccatgcttcgtgtaaaacccaatcccgaaggccgtttagagccttttgccgtcgtaaaagcaggtgtgcgaatcttttcagatggccgcaacccgaaagatggttcctcattagccgtgacccgccgcgaagcgcgtgccatgcgccgccgccgcgaccgcttactgaaacgcaaagcacgtatgctgcaacagctcactgcctttggctttttccccaccgatctggccgagcgcaaagcactggaaacgctcaacccctacgaactccgcgccaaaggcctagatgaaccactcagcccttatgagtttggccggtcgctcttccacattaaccaacgccgtggctttaaaagtaaccgcaaaaccgacaaaaaagaaaacgatagcagcgccctcaaagccgccattcggcgtgtggcgagcgaaatcgacggcaatcaggcgcgtaccgtcggcgaatggctctacaagcggatgctcaatggtcagcccgtaagaggtcggtatcgcgaaaccaaggtacaaaaagaagacggcaaaaccaaaatcgataaaagctacgatctctacatcgaccgcgccatggtagaggccgaatttgaagcgctctgggccaagcaagcctcgctaaatcccgccgtctacagcgagcaagccaaggccacgctaaaagatgttttactgttccaacgcaatttgcgcccagttaaacctggccgctgcacactgattcccactgaagaacgtgcgccactggccctaccgagtacgcaacgctttcggatttatcaagaagtaaataacctgcgcatactacgtgaaggtttaaaagacgaagcacttacactggtgcagcgtgatgcgctcgtcaccgcgctggaacagaacaacaagcggacttttgctcaaattaaaaaattgctcggtcttgatgggcaaacccaatttaattttgaagaccccaaacgccaagaactcaaaggcaataccaccagcgctattctgagccaccccaaacattttggtgacgcttggtttggttttgatgaagccaagcaagacgggattgtgtgtcagcttctcaacgaagaaaacgaaagcgcgctgattcgctggctgatggatcacacaggggtggatgaggcccatgccgaagctattgccaatgcggcactccccgaaggctatggctcgctcagccgcgctgccctcgccaagattctgcccgagctccgcaaagcggtgattacctatgacaaagcggcgcaagccgccgggtttgaccaccacagccacattagccccagtaccaccggcgaaattttgcccgaacttccctattacggcgaggcgctgcaacgccatgtgggctttggcacgggcaaccccgatgatgtgcctgaaaaacgctacggcaaaatcgccaaccccaccgtacacattggtttaaaccaagtgcgcaaggtggtgaatgcgctcattaaacgctatgggcaccccagcgaggtgattgtagaagtggcacgcgacctcaagcaaagcaaaaagcagcgcgatgaagaaaacaaacgccaggccgaaaaccaaaaacgtaatgagcggattcgtcaagatattgccgccatgcgtcctgatagaagtgaagagcgcgtgacccgtacagatattcaaaaatggatcttatgggaagagctaagctttgatccagcgaatcgatgctgtccctactcgggcgtacaaatcagcgctgaaatgctaatgagtgatgcggtggaaatcgaacacatcttgcccttctctcgcaccttggatgacagcctgaataacaaaacggtgtccatgcgccaagccaaccgcattaaaggcaaccaaacaccctttgaggcctttggtaaagacaatgctctgggcgtgaactacagcgacattctgatgcgcgcgcagcagatgcccaaagccaagcgcaaacgtttcgctgaaaatgctttggaagaatggctgcaaaatgaaaaaaattttcttgcccgcgcgctgaacgacacccgctacctcagccgcgtggcacgcgaatacgtcagcctgatttgcccacaagccacacgcgtgattccgggacaaatgacggcacagctccgcgccaaatttggcctgaatgacatactcggtttagatggcgaaaagaaccgcaacgatcaccgccaccatgccgttgatgcctgcgtgattggcgtcaccgaccaaggcttgctccagcgatttgcctatgccagcgccagcgcccgcgcaaatggactagcgcgtttggtagacaccatgcctgacccttggcccagctaccgccagcacgtacaacgggcggtacaaaatatttatgtcagccacaaacccgaccacagccatgaaggcgcaatgtttgacgaaaccatctacagcgccactgggaaaagtcgcagcgcagctaaagatcgcacagtcattccttttattgctaaaaattggagccatcctgacgaccacaacaaacagcgtccgttcaagggcttgataacagatgtttctcaacggcatcaaaacaaaccctacaaaggattactatcaaacagcaattactgcatcgaaatttattcggatgaagcaggctggggtgggcatgtactcaaaacttttgatgcctatcaaatcgtcagggcacacaaaaatgctagtgagggtatgcaagcgctacgtaataaacatagcagccagaatgggcacccactagttatgcgtttgatgatcggggactacattcgcgcagaaatagatggtttcttattgctgctccaagtacttaaaatcaatagttcaggctcgatcaccttcattaagccgaatgaaaccaatatcagcgcacggtacttagcaaagctagcagcacaaaaagctcaaaaagaaggtaagccattcgatgacattgcgctgaatgatgttttctttcagaaagcaatatctgccgatagtttacggttatttaaagctaggcctgtcaccctctcccccatcggtgagctacgcgaccccggctttaaaggc

SEQ ID NO: 16 - DNA, 3213 bp, Pelagicola sp.
  source 1..3213: mol_type = genomic DNA; note = LXJ1103
atgcgacttggccttgatattggaacgaactccatcggctggtggctttaccggacggaaaatgaccagataacgtgcgtcgtcgatggcggagtgagggttttctcggatgggcgggatccgcagtcaaaggagtcgctggccgtcgataggcgcgtggcccgtgcgcagcgccgtcgccgtgacagatacttgcgccgcaaggctgctttgatgaaacggatggctgaggcaggcttgatgccagccgatcccgtgcaggccaaagcgttgcaagctcttgatccctatgaccttcgggcacgggggctcgatgaggctttgccgctggcgcatttcgggcgggcgctgtttcacctcaaccagcgacgtgggttcaaatccaatcgaaaggccgaccgcggcgataacgaaagtggtaagatcaaggatgcgactgcacggctggattgggcgatgcgcgatgcacgcgcacgcacctatggcgagtttctgcatatgcggcaggacaaggccgatgatccgcgtcgcgtcccgacggtgcgcacgcgcttgtccgtggcgcggcgtgacaatgccgaaaaagaggaggcagggtatgatttttaccccgaccggcggcacctgtcagaggagtttgacgcgctatgggccgcacaggcggaacatcacaccaccctgacggacgatctgcgcgaccagatcaagacgatcatctttcaccagcgccccctgaaggcgcctgaggtcggtctgtgtctattcacggatgaacggcgtattccgtctgcgcatcccttgaaccagcggcggatcctgttggagacggtgaatgggctgcgaattgttgcgcgaggggaggcggcacgtgggttgactcgtgaagaacgtgaccagatcgttcatgggctagataacaaaggacatacgaaaaccctgtcgggaatgtcgatgaaactcagggcaattggcaaggtgatcaaacttaggagtgaccaaagtttcacgttggagactgccaaccgggatgccattgcctgcgaccccgtccgtgctagcctgtcgcatccagaaaggatgggcggggtctggaccacactggacgaagacgctcaatgggacgtggtgcaacgcctccgtgcggtgcagagtgataccgagcatgaggcgctggttgattggttgatggccacccacgggcttggccaggattatgcgcaagcaacggcgaatgcgccgctgcccgaaggttacggacggcttgggctgacggcaacgcgcaagattctggcggcattggaggctgatgtgatgtcctatagcgatgctgtggccgcctgtggttggtcccattctggcggaccaacgggtgaggtgttggaggctctgccatactacggtgaaattctggaccggcatgtaatcccgggaacaggtgtgaaaactgacgaggacgtcaaacgcttcggtcgtattaccaaccctacggtccatatcgggttgaaccagatcaggcgactggtgaaccggatcatttgtgtgcacggcaagccggaccagatcgtggtggaggttgcccgcgacctaaagaactccgaggatcaaaagcgtgagattcagaagacgatcaggaaaaatacggatgacgcgatcaagcgcggcaaaaagctggttgaagaactggggcaaaaagacacaggcgcaaaccggttaatcctgcggctgtgggaaaaccttggcaatgacgttatgacccgccaatgcccctattctggcaagcgtatcagtgcggcgatgttgtttgacgggtcgtgtgatgttgatcatatccttcctttctcacgtacgttggatgatagcatttggaacaagaccctatgcttgaaagaggaaaatcggaaaaaggctaacaagacgccgtgggaggtctggggcgaaacagaccagtgggacgtgatcgtcgccaacctcaagaatcttgatagaaagcaggcatggcgctttgcccccgatgcggtggaacggtttgagggcgagaacgacttttccgcccgtgcactaaaagatacccaatacctgtctcgtgtggcacgggcttatctggatgcgttgtatgacggggcggacggcaaatcccatgtttgggttgtgccgggccggttgacggaaatgctgcgccgccattgggggttgaacgggatcgaggtgctgacggacagcgacgcgcaaaccgtgaaatccaagaaccggcaggaccaccggcatcacgccatcgacgccgccgtggtcgcagccaccgaccgcagcctgatccagcggatcagcaagatagccaagcacgacgaacaggcgggcgccgaacaggtcgcgcggtccgtcccgccgccttgggacggtttccgtgatgatgtcgccggacagatcgggcggatcatcgtcagccacagggcagatcacgggcgcattgatccgaccgcgcgcgcacaaggcagtgacacgacagcggggcagttgcatatggatacggcttacgggatcgttgacggtgggcatgtggtcagccgcaaaccgctgatgtcgcttggtgcaggtgatatcaggaaaatccgcgatcccgatttgcagcgtcatctcacgcgtgtcacgcgcggtttggacaagaaggaatttgaacaggctctggcctcttttgcggcgtcgcgcaaactgccggaccaaagcgaaaacccttatttcgggctgcgacgtgtgcggcttctggatgccttgcaagacagtgcccgtgtgccggttagaaacgccacaggcaatatctacaaggcctacaaggcaggtagcaaccattgctacgaggtgtggcggatgcctgatggcaaggtcaagccttgggcaatctcaacgtttgaggcgcaccaatccggcgatgggtccaagccccatccggctgcaaaacggctcttgcgggtgttcaagcgggatatggtggtaattgaacgtaaagggataacggtgatctgctacgtccagaaaatggacgttgccaacggcctgtttctggtgccgcataccgaggggaatgccgatgcgcgcaaccgcgacaaggaggatgatttcaaattcatccagatgtctgcggcgtctttgattaaggccagaatccgccgcgttcacgttgatgaaatgggccggatgcgcgacccgggcccgccccgt

SEQ ID NO: 17 - DNA, 3144 bp, synthetic construct
  misc_feature 1..3144: note = OMNI-59 DNA optimized for expression in human cells
  source 1..3144: mol_type = other DNA
aacgtgtggaaaggcaccggcttcgtgtcccggaagcggaacatcatcagatacagactggccctggatctgggcagcacatctcttggatgggccatcctgagactgaacgccgataatcagcccaccgccattctgaaagccggcgtgcggatcttcagcgacggcagaaatcctaaggatggcagcagcctggccgtgactagaagggctgctagagccatgcggcggagaagagacagactgctgaagcggaaggcccggatgctggataagctgatcgcccacggattcttcccacaagacgaagccgccagaaaggccctggaagtgctgaacccttatcagctgagagccgagggactgcagagggctcttatgcctggcgaatttgccagagccatgttccacatcaaccagcggagaggcttcaagagcaacagaaagaccgacaagaaggacagcgactctggcgccctgaaaacagccattagccagctgagacagcagctgcagaacgagaatgccagaacagttggcgagtggctgtggcagagactgcaagctggacagggcaccagagccagatacagggaaaccagaatcgccaccgataccggcaagggcaagatcgacaagagctacgacctgtacatcgaccggcagatggtggccgatgagtttgatgctctgtgggccgtgcaggccgccttcaatcctgtgctgtttaatgagcaggccagagccgagctgagagacactctgctgcatcagaggccactgaggcctgccaagcctggaagatgtacactgctgcccgaggaagaaagagccccactggctctgcctagcacacagcggttcagaatcctgcaagaagtgaaccacctgaggattctgcaccccgacctgagagaagaggccctgacactggatcggagaaatgccattgtggccctgctggaaaaccggggcaaagtgacattccaggccatgcggagaacactgaacctgggagatgccgtgcagttcaacctcgaggacgccaagagaagagagctgaagggcaacgccacaaccgccgctctgagcaagaaagaactgtttggagccgcctggcacgacttcgatgaagccctgcaggatgacatcgtgctgagactggtcacagaggaatctgaggccgaactggtgcagtggctgatcgagaatactggcgtggacgaagccagagctaccgccattgctaataccggcctgcctgaaggctacggcagcctgtctagaaaagccctggccagaatcgtgccagctctgagggccgaagtgatcacctacgataaggctgtgcaggcagccggctttgcccaccactctgatctgagattcagcttcgagtacgacagcgccgacgtggaacaagtgggcgagagaatcgataagaccaccggcgagattctgcccgtgtccgcctttaagcagctgccttattatggcaaggccctgcagagacacgtggcctttggatctggaaaccctcaggaccccgaggaaaagagatacggcaagattgccaatcctaccgtgcacatcggcctgaatcaagtgcggagagtggtcaacgacctgatcagaagatatggcagacccaccgagatcgtggtggaactggccagagaactgaagcagagcagagagcagaagctggaagcccagagaaagcaggccgacaaccagaagagaaacgccagaatcagggccgagatcgcccctatcctgggcatctctgaggaaagagtgaagcacgccgacatccaaaagtggattctctgggaagaactgagcttcgacgtggccgacagaagatgtccttacagcggagtgcagatcagcgccagaatgctgctgagcgacgaggtggaaatcgagcacatcctgcctttcagccagacactggacgacagcctgaacaacaagaccgtgtccatgcggcaggccaaccggatcaagggcaatagaacaccctggcaggccaggcaggattttgaagcccaaggctggccttacgagggaatgcagcaacgggccgaaagaatgcccagagccaagagatacagattcgcccctgacggctatgagcggtggctgggagaagatcagggattcctggctagagccctgaacgacaccagatacctgagccggatcgccagagactacctgacacttgtgtgtcctggcggcgttagagtgatccctggaagaatgacagccctgctgagggctaagtttggcctgaatggcgtgctgagcctgtccggcgagaagaatagagatgaccacagacaccacgccgtggatgcctgtgtgattggagtgactgatcagggcctgctgcagagatttgccgaagcctctgccatggccagacagcagggactcgagaagctggtggaaaccatgcctctgccttgggagacataccccgctcatgtgcagagagctgtgcagaacatctgggtgtcccacagaccagatcacggccacgaaggcggcatgatggaagagacaagctacggcatcagcaaggacggcaggatcaagcagagaagaaaggccgatggcagccagggcagagagatcagcaacctgattcggatcagcgagccctctcagcctgaaagacatggcgttgacgccgaaggccaaccactgccttacaagggctatgtcggcggcagcaactactgcatcgagatcacccggaacgacaaaggcaagtgggaaggcgaagtgattagcaccttccgggcctaccagattgtgcggaaacatggcgtggccagactgaggcatcccgagatggctcagaatgggaaagccctcgtgatgcggctgatgatcgatgactgcgtgcggctggaactggatggccgggaagaaaccatgagagtggttgtgatccccagaaacggccaggtgttcatggcccctctgcacgaagccaacgtggacgccagaaacagagacaagaacgaccctttcagctacatctccaagatggccggcagcttcctgaaggccaaagccaggcacatcacaatcagccctatcggcgagctgcacgaccctggctttaaaggc

SEQ ID NO: 18 - DNA, 3060 bp, synthetic construct
  misc_feature 1..3060: note = OMNI-61 DNA optimized for expression in human cells
  source 1..3060: mol_type = other DNA
accggaacagatgccaccgctcacagccacacaccttacagactgggcctcgatgtcggcacaggatctcttggatgggccgtcgtggaactggacaccgatggaaatcctgtgcggatcgtgcggaccggcagcagaatctatggcagcggcagaaagcccaaggactttagctctctggccgccgatagaagggccgccagacagatgagaaagcagcgggatagatacctgcagcggcggacaagactgatgcatgaactggtggctgccggactgatgcctgaggctgaggtggaaagacagaagctgaaggatctgaacccctacctgctgagagccagaggcgtgaaagaagaactgacagcccacgaactgggcagagccctgtttcatctgcaacagcggagaggcttcaagagcaacagaaagaccgaccggaaggacgacgacagatccgccatgaagtctgccattgccagcctgcagtctgacctgggagatgatacactgggcacctacatgtggaagagaatccagaatggcgagagcgtgcggagcagacctagaaaagtgggcagcaagaacgagtacgacttctacgtgaaccgcgccatggtggaagatgagttcaaccagctgtgggactaccagagccagtctcacggcgatctgctgaccgatgaggccagaattagagtgcacgacgccatctttagccagcggcctctgaagcctgtggaccctggcagatgcaccttcgagacagatcagagaagggcccctaaggctctgcctagcagccagctgttccggatctaccaagagctgaacgccatcagagtgatcgacccctttagcagcgaccaggccgatagacctctgaccagacaagaaagagatgccggcgctagtttcctgctgggaagagtgaaggccacctttccacagctgaagaaagccatgttcggccccaccaaactgcagctgtctctggaatacggcgagcggaagaatatcctgggcgacgttgtgggctctgagctgagaaaagcccagcacatcggccccgactgggagacatatgatctggccactcaggacctgatcgtgaccatcctgctggaagccgacaccgacgacgaagtgattgaaaggctgcaggccgagagcagcctgtcactggatcaagttcatggcgccctggaagcccctctgcctgatgattatctgagactgagccacagagccatcggcaagatcctgcctcacctgaaggacgagtggaacgaagagggcaacgcccctgtgatgtatgatgccgctgttagagccgccggatatcagagccacagcgagtttcatagcggcgtgctggaagataccctgccttactacggcaaggtgctgaagcggtacacacaagaggtgtccggatcttcccaggccgccacaaatcctgatgagtgggagtttggcaagatcgccaatcctaccgtgcacatcggactgaaccagatcagaaccgtggtcaacagcctgatcgacagatacggcctgcctacacagattcacgtcgaggtggcaagagatctgggccagtctgccgagggaagaagagaggctgccagcaacagagccaagaatgagagagccaacgaggccctgaatgccagactgacagagctgggccagcgcaccaatttcgccaacagagagagactgcggctgtacgacgagatcagcgtgctgaatcacagatgcgtgctgacaggcatccccatcgagatgagcagactgttcaccaacgactaccaggtggaccacatcctgcctttcagcagaaccctggacgacagccggggcaacaagattctggtgcatcacaccgccaaccagttcaagggcgccagatctccattcgaggcctactctgagacagccgattgggatcacattctgcagagagcctctgacgcctttggcgccacctctcctaagttcaagagattcagcgccgacgccatggacagatacagcaacggcgagcaggactttatcgccaggcagctgaacgacaccagctacctggccagagtgacaagagagtacctgggcagcatcgtggaccccgacagaatccttgctacacctggcagactgactagcctgctgaggcaccattggggcctgaatggactgctgtctgacgccgccgagaagaacagaagcgatcacagacaccacgccatcgatgctctggtggtggccctgtctgagagagtgacactgaaagccgtgaccgacgctaatcggagagccggcgatcagggaatcgaaagactgctggttgacctgcctcagccttgggagggatttgccgatcacgccagagaaagcgtggaccggattgtggtgtcccacaagcctgaccacaacgagaaaggccagctgcacgaagagacagcctatggcgttctggaaggccccgacaagaagggcagattcctgaccagaaagcggatcaccgatcctgccaagggcgttgtcggatcttgggaacagcctaagtggcgggacgtgatcccactgtatagaagaggcgagggccctgacagcacactgccttacaaggcctacatcggcggcagcaactactgcatcgaaattgtgcggacagccaaaggcaagtgggctggcgaagtggtgtctacccatacagccaacacagccgagtaccgggcctttatggctgagcctggtgcctacagagcccagtcttatgctggcggcgatctcgtgatgaggctgatcgccaacgacacaatcgccatcgaagtgggagatgctggccggcagatcatgagactgtgtcagctggaaaccgtgggagccatgtacttcgccaacgttcgcgagggaaacgtggccgcaagaagcagagccagggacaacgatttcagtctgctgaagaaggccgcctccacactgcagccactgaaggctagaagagtgtttgtggaccccatcggcagagtgttcgatcccgacttcaaagag

SEQ ID NO: 19 - DNA, 3135 bp, synthetic construct
  misc_feature 1..3135: note = OMNI-67 DNA optimized for expression in human cells
  source 1..3135: mol_type = other DNA
aactacatcctgggcctcgatctgggatctgcctctcttggatgggccgtgctggaatgtaccgaggttgacggaagcctgcagcctaccagaatcgaaagaaccggcgtgcggatctttgaagccggcgtggaaggcgatatcgagcagggaagagatgcctctagagccgccaagagaagagaggccagacagcccagacggcagaattggagaacccagcagcggaagcggaagctgtttagactgctgcaacagcacggactgctgcccgcctctgagaaggatgatgccatcagcagaaaggccgtgttcgaccagctggacaaagagctgaccgagaagcacatcaccgagggcgatcacacagcccaccagcatctgccttacctgctgagaatgctggcctccggcgccaaagtgaagccttttgaactgggcagagccatctacagcctggctcagagaagaggcttcctgagcaataggaaggccgacaccgacgagaaagaggatggcgtcgtcaaggcctctatcagcgaactcggaggacagatcgccggcagaacaatcgcccagaccttcgtggaagatatcagccccgatcacgaggaccccggcagacagagaatcagacagaggtacaccgccagagagatgttccacgacgagttcaaccggatcagaaagcagcagcagccccacttcgacctggccgacaatgattgggacaccgtgtacaagaccatcttcttccagcggcctctgaagtcccagcggcacagaatcggaagatgcgagatcgatggcggccagagatgtctggatgccctggatgtgttccagcagttcagaatctggcacgccgtgcagaacctgagactggccgatgcctattctctggggagagatggcagactgaccctggaagaacagcagaaaatcgtggacgccctgcagacccaggccaccatgacatggggaaaagtggttaccctgctgggcctgaagcggggcaccaagtttacaatccaagagtggaacaccaagggcctgaccggccacagaacaaactctgccatgatgcacgtgttcggcgacgagtggctggacagacctctggaagagagggacgccatcaccaaagaagtggtgtacttcagaaagcccagcgccatgagaaagcggggacaagaagcttggggcctgtctgaagaacaggccgctctgctgcctagcaccagacttgaagaagcccacgccagacattctgctgccacactggccatctttgtggaacggatgagcagaggcgaggactacagcaccatcagaaaggacatcaccggcaaggacgacagcgagcctcttgatcaactgcctcctctgagcaaggccggactggatatcaccaatcctgccgtgatcagaggcctgacagagctgagaaaggtggtcaacgaactcgtgcggcagtacggcaagcctatcggaatcagaatcgagctgagcagaagcctgaagaactcccgggacaagcggatcaagctgcacaaggacaacgaggaccgcagaaagcggagagagaaggccatcgagggaatcctgaagcagatccctggcagatacagcggcaacgacatcgagaaatggctgctggccgaggaatgtggctggcactgtccttataccggcagacctatcagccctagcacactgctgggcagccagcctcagtttgacatcgagcacatcttccctcggcgctacctggacaacagcttcagcaacaagaccctgtgctaccacgagtttaatcggaacgtgaagaagaaccagaccgccttcgatgcctgtagcggcctggattcttgggacgagatcctgcagagagtgaacaacttcgacggacccgtggctgccctgaagagaaagagatttctgaccgccgccaaagagatccccgacggctttaccagcaagcacctgaacgacaaccggtacaacgccgtggtggccaagaaatacgtggccatgctgtatggcggcctgtccgatgctgatggatctcagagagtgtttgccgtcaccggcggacatacagcactgcttagaagagagtggggcctgaacagcatcctgagcggcaccgaagagaaaaccagggacgatcacagacaccacgccgtggatgccgtggttattgccctgacagatcctgccaggattcaggccctggttaatgccgccgagctggccgaaaagaaggccagcagacggttttatgaggccgtgcaggacccctggcctaagtttagctctaaggtggccgacagcatcaacgagattgtggtgtcccaccggcctacaagaacactgcctggtgctctgcacgccgagagcatctactccaagcctcacatcgacaaggacggcaacaccaaccacaggattcggaagcacattaccaagctgtccgccaccgagctgaagaaagacaagatcgtggaccccgccatcagggacctcgtgaaggccaaactgaaagagctgggcgagagcaaccctgccaaggcctttgccgaggaaaagaatcacccatttctgacagccaaggatggccggaagatccccattcacaaagtgcgggtgttcgccgataagaagcctcgggccattgccaagaacgagcggcagagatatgtggccagcggcaaggatagcaacttcgccagcatgatctacgctgtggtggataaggacggccacgagatcaagtgggagcacaaagtgatcacccggctggaagcccatgagagaaagacccggaacagaaccgtgaacggcgagaaggtgctgctgcctgatcctaccgacttcaacgacgacaagaaacgggtgttcaagttcgccctgtgcaagaacgacaccgtgatgctggaaggcccagatggcgacgacgtgatctgcagaatccagaagatcagccaggccgagatccagctgtgtcctctggctacacctagcgtgcagggcaaagccagatccaagtggaaccagatccagagcatcgacaacctgcggaagtggaatctgcggaccgtgctgatctctcccaccggaattgagcacaga

SEQ ID NO: 20 - DNA, 3105 bp, synthetic construct
  misc_feature 1..3105: note = OMNI-76 DNA optimized for expression in human cells
  source 1..3105: mol_type = other DNA
ctgcacaagatgagatacagactggccctggatctgggcagcacatctctcggatgggccatgatcagactggacgccaatcagaggccttgcgccgtgattaaggccggcgtgcggatcttcagcaacggcagaaatcctaaggacggcagcagcctggccgtgactagaagagaagccagagccatgcggcggagaagagacagactgctgaagcggaaggcccggatgatgagaaccctgatcgagtacggattcttcccagccgaggaagcccagagaaaggccctggaaacactgaacccctacaagctgagagccgacggcctggataaggctctgacaccagccgagttcggcagagtgctgttccacatcaaccagcggagaggcttcaagagcaacagaaagaccgacaagaaggacaccgactctggcgccctgaaaaccgccatcagcaagctgcgcgagatcctgaaaacagagaactgcagaaccgtcggcgagtggctgcacaaaagaaatcaggccggccagacagtgcgggccagatatagacaggacaagaccatcaaggacgacggcaaggccaagatcgacaagtactacgacctgtacatcgaccgcgccatgatcgagcacgagttcaatgagctgtggcggaagcaggccgagttcaaccctgctctgtttagcagcgccgcctacaccgatctgaaggacgttctgctgtaccagcggcctctgaagcctgtgaagcccggcagatgtaccttcatgagcgacgaggaaagagcccctctggctctgcctagcacacagcggttccggatgtaccaagaagtgaacaacctgagaatcctgcgcgagggactgaaagaggaacccctgacactgcagcagagggacgatctgatcgtggtgctggaacggaacaacaagcggaccttcacacagatcaagaagctgctcggagttggcggagccgtgcagttcaatttcgaggaccccaagagagaggaactgaagggcaacaccaccaacgccatcctgggcaagaaagagcactttggagaggcctggatcgccttcgatgaggccaaacaggatgccatcgtgatgcagctgatcaaagaggaaaacgaggccaagctgatccagtggctccaggacgagacaggcattgaggaagagagagccgagatcattgccaacgtgggcctgcctgaaggctatggctctctgggaacaaaggctctggccaggattctgcccgagctgagaagagatgtggtcacctacgataaggccgtgcaggctgccggatttgagcaccactccaagctgaatcagaaccggggcatccccggcatcaccttcaagatcgagagcatcgatcaggacaccggcgaaatcaaagagtttcacatccacaaagagctgccctactacggcgagtacctgcagagacacgttggctttggcagcggaaagcctgaggaccctatcgagaaaagatacggcaagatcgccaatcctaccgtgcacatcggcctgaatcaagtgcggctggctgtgaacgccctgatcaagagatacggacaccccagcgaagtgatcgtggaagtggccagagatctgaagcagagcaaagagcagcggagcgaagagaacaagagacaggccgagaaccagcagcggaacaatcggctgagaaccgagattgcccgcatcctgcagatcaacgaggaaggcatcagacgggacgacatcgagaagatgatcctgtggatcgagctgagcgccgatgtggccgatagaaagtgtccttacagcggcgtgccaatcagcgccaccatgctgctgtctgacgaggtggaaatcgagcacatcctgcctttcagccagacactggacgacagcctgaacaacaaaacagtggccctgagaaaagccaacagagtgaagggcgacagaaccccttgggaagctcagcaggattttgccgctcaaggctggtcctacgccgacattctggctcgggccgagaatatgcggaaagagaagcggtacagattcgccgaggacggctacaagagatggctgaaggatgacgccggcttcctgcctagagcactgaacgataccagatacctgagcagagtggcccgggaatacctgcacctgatctgccccaataccagagtgatccctggcagaatcaccgccatgctgagatcccagttcggactgaacaaggtgctgggcctgaacggcgagaagaaccggaatgatcacagacaccacgccgtggatgcctgtgtgatcggagttacagatcagggcctgctgcagaagtttgccaaggcttctgccagcgccagagagaagcagctgaacagactggtggacaacatggaaagcccttggaagaactaccaagagcacgtgcagagggccatcgatgccatctgggtgtcccacaagcctgatcattctcacgagggcgccatgcacaatgataccgcctatggcctgagaggcaacggcaaggtgtcctttcacaaaatgatggacggcaaacgcgagtacatcgaggacaacctgaaagtgatcgagatcgccgataccaaggccgccgaaagacacggactgctgcctaatggcaagcccaagccttacaagggctacaaaggcgacagcaactactgcatcgagattgtgcggaacgagaaaggcagatgggagggcgaagtcatcagcacctttgaggcctaccagctcgttagagaacagggcgctgcacagctgagacacccagctctgggaatctctggcaagcctctggtcatgcggctgatgatcgatgacaccgtcaggctggatgtggacggccagagctgtaccatgagaatcgccaagctgtcctccaacggccagatcttcatggccgacatctgcgaggccaatgtggacgcccggaacagaaacaaagaggactccttcgcctacatctccaagatggccggctctctgcagaccgccaaggccagaagagtgacaatctctcccatcggagagctgagagacagcggcttcaaaggc

SEQ ID NO: 21 - DNA, 3183 bp, synthetic construct
  misc_feature 1..3183: note = OMNI-79 DNA optimized for expression in human cells
  source 1..3183: mol_type = other DNA
gagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggat

SEQ ID NO: 22 - DNA, 3222 bp, synthetic construct
  misc_feature 1..3222: note = OMNI-80 DNA optimized for expression in human cells
  source 1..3222: mol_type = other DNA
accaccacactgggcatcgatctgggcacatcttctctcggctggtgcctgatcgaggacgagcacagaatcctggacctgggcgtgatcattttttctgccgctgccggcgctggaagagatcctcaatctggtgctcctctggccgaggccagaagagaagccagaagcgctcggcggagaagagacagattcatcggcagaagaagcgccctgctggacaagctgattgctctgggacttctgcctggcgatccaccagctggacacggaagaagaagaaatcaggccctgcctaacgccgagacaaaggcccttgccgatacagatccccacgtgctgagaagaagggctctgtctgaacctctgagccctcacgagatcggcagagccatcttccacctgaacaccagacggggcttcaagagcaacagaaaggccgacagaggccggaatgagcccgagacaggcaaaattgctacagccggacaggccctggatgccgctctgggaaaaagaacactgggccagtttctggccgatcggattgatgctggacagcctgccagagttagaatgggcggcgagaatcaggcctacgacttctatcctcagcggagccacctggaagccgagtttgctgccatttgggaagcccaagagcaccaccatcctgagctgctgaccgatacagccagaaccgccatccaccgcatcctgttcttccagaggccactgaaaacccctgaagtgggcttctgcacctttgccggaatgtctggcgtgccccatgatgagagaaggctgcctaaggctcaccctctgttccaagagcggagactgtacgaggaagtgaacaacctgaaagtggtggccgctggcgctgccgctagagatcttacactggacgagcgggacagactgatcctgaagctgcgggacaacaagaaagtgaccttcgccacactggccaagaaggtgctgaagctggccgaaggcgagcggttcaacaaagagagcgaggctagaaaggacctggctggggatgaagtgcgggccgaaatggccgataagaagagattcggcaaccggtggacccactttccactcgagagacagctgcagatcatcgaccgggtgcagaacgaggaaaaccccgatattctgctggcctggctgcagagcgattgcggacttgataaggctgctgctgtggccgtggccaggacaaatcttcctgaaggccacggcagattcggcgagacagccacaagaaggctgatcgccgctctgaaggccgaggtggtcacatatgataaggccgcactggctgccggcttccaccactctgatcatagaaccggcgaggtgtacgacctgctgccttattatggcgaggtgctgaccagagagatcgcccctggaaaagccgagtatggcgaccctctggaacggcagtacggcaaagtgaccaatcctaccgtgcacatcggcctgaggcagctccagaaactcgtgaatgccgtgatcgccagacacggcagacccgacagaattgtgattgagctggcccgggaactgaagctgaacgacaagcagaaagacgagcaccagcggcggatcagaagagataccgaggccgccattagacggggcgagaaactggttgaagccggaattgctgacaccggcgccaatagagcactgatgagacagtgggaagaactgaaccccagcaatcccctggacagaagatgtccttactgcggcgagcctatcggcatggcccagatctttaacagcctggccgacatcgaccacatcatcccttacagcagaagcctggacgacagccccgctaacaaagtgctggtgcaccggaactgcaaccggcagaagggaaacaagaccccttgggacagatggcacgaggatgaggccaagtgggagatcatttctgcccaggtggccagaatgcaccctagcaaacaatggcgcttcggccccgatgccatggaaagacttgagagagatggcggctttgccgccagacagctgaccgacacacagtacctggctcggatcgccgacaagtacctgagaggactgtaccctacagccgacgagggcagagtggatgtgatccctggaagaatgaccgccatgctgcgaagagtgtggggcctcaattctctgctgcccgaccacaacttcgtggaaaacgagcactctagcgcccctaagaaccggctggatcacagacaccacgccattgatgctacagtggccgccgtgacaagcctgagcagaatgcaacagattgccgccgcagccgccagatccgaggaaaaagagctggaaaggctgttcgacgatctgccccatccttgggacggctttagagaggatctgggcgcctgtctggctagaacagtggccacacacaagcccgatcacggaagatctgccaagccaagcagacacagagatgtgacagccggcaagctgcacaatgataccgcctatggcctgaccggcctgaaaaccaccgatggcaagacacccatcgtggtgcatagagtgctgctggcttccctgaagcctacacagatcgccgatcctgactgcatccccgacgagacactgagaaatgccctgtggctggccacaagagattgcagcggcaaggcttttgaccaggcactggccagattcgccaaagaacaccccgtgttcaagggcattagaagagtgcggatcagggaaccactgaacgtgatccccatccacgacagagagggcaagccctacaaaagctacgctggcgcctccaacgacagatacaatgtgtggcggatgcccgacgcctcttggagacacgatgtcgtgtccaccttcaacgcccacagaagcgactacagagatctgaggccacatcctgccgccaaaaaagtgctgtccctgcggcagaacgacatgatcgccgtggaaagaaacggcggcctgcgcgagatcatgcgggtcgtgaagtttaatcaggccggcagactgaccctgtgtcctcctaatgaaggcggaaagctgcagaacagggacgccgctccaaacgacgccgatccattcaagtacacctatctgagccccagcagcctgaagaacgccaaagccagacaagtgcgcatcgacccactgggaagagtgttcgatcccggacctagagag

SEQ ID NO: 23 - DNA, 3132 bp, synthetic construct
  misc_feature 1..3132: note = OMNI-81 DNA optimized for expression in human cells
  source 1..3132: mol_type = other DNA
aagaacaagatgcagtaccggctggccctggatctgggcacaacatctcttggatgggccatgctgagagtgaagcccaatcctgagggcagactggaaccttttgccgtggttaaggccggcgtgcggatcttctctgacggcagaaatcctaaggacggcagcagcctggccgtgactagaagagaagccagagccatgcggcggagaagagacagactgctgaagcggaaagcccggatgctgcaacagctgaccgccttcggcttctttcctaccgatctggccgagcggaaggccctggaaacactgaatccttacgagctgagagccaaaggcctggacgagcctctgagcccttacgagtttggcagaagcctgttccacatcaaccagcggagaggcttcaagagcaaccgcaagaccgacaagaaagagaacgacagctctgccctgaaggccgccattagaagagtggccagcgagatcgatggcaaccaggccagaacagtcggcgagtggctgtacaagagaatgctgaacggccagcctgtgcggggcagatacagagaaacaaaggtgcagaaagaggacggcaagaccaagatcgacaagtcctacgacctgtacatcgatagagccatggtggaagccgagtttgaggccctgtgggctaagcaggcttctctgaatcctgccgtgtacagcgagcaggccaaggccacactgaaggatgtgctgctgttccagagaaacctgcggcctgtgaagcctggcagatgcacactgatccccacagaggaaagagcccctctggctctgcctagcacacagcggttccggatctaccaagaagtgaacaacctgagaatcctgagagagggcctgaaggacgaagccctgacactggttcagagagatgccctggttacagccctggaacagaacaacaagcggacattcgcccagatcaagaagctgctgggactcgacggccagacacagttcaacttcgaggaccccaagcggcaagagctgaagggcaataccacaagcgccatcctgtctcaccctaagcacttcggcgacgcttggtttggcttcgacgaggccaagcaggatggcatcgtgtgccagctgctgaacgaggaaaatgagagcgccctgatccggtggctgatggatcatacaggcgtggacgaagctcacgccgaggctattgctaatgccgctctgccagaaggctacggctctctgtctagagctgccctggccaagattctgcctgagctgagaaaggccgtgatcacctacgataaggctgcccaggccgctggctttgatcaccactctcacatcagccctagcaccacaggcgagatcctgccagagctgccttattatggcgaggccctgcagagacacgttggctttggaaccggcaatcccgacgatgtgcctgagaagagatacggcaagatcgccaatccaaccgtgcacatcggcctgaatcaagtgcggaaggtggtcaatgccctgatcaagagatatggacaccccagcgaagtgatcgtggaagtggccagagatctgaagcagagcaagaagcagcgcgacgaagagaacaagagacaggccgagaaccagaagcggaacgagcggatcagacaggatattgccgccatgaggcccgacagatccgaagagagagtgacccggaccgacatccagaaatggattctgtgggaagaactgagcttcgaccccgccaacagatgctgtccttactctggcgtgcagatcagcgccgagatgctgatgtctgacgccgtggaaatcgagcacattctgcctttcagcagaaccctggacgacagcctgaacaacaaaaccgtgtccatgaggcaggccaatcggatcaagggcaatcagacccctttcgaggccttcggcaaggataacgccctgggcgtgaactacagcgacatcctgatgagagcccagcagatgcccaaggccaagagaaagagattcgccgagaacgccctcgaggaatggctgcagaacgagaagaacttcctggctagagccctgaacgacaccagatacctttccagagtggcccgggaatacgtgtccctgatctgtcctcaagccaccagagtgatccctggccagatgacagcacagctgagggccaagtttggcctgaacgatatcctcggcctggatggcgagaagaatcggaacgatcacagacaccatgccgtggatgcctgtgtgatcggagtgacagatcaaggcctgctgcagagatttgcctacgcctctgccagcgccagagctaatggactggctagactggtggacacaatgcccgatccttggcctagctacagacagcacgtgcagagggccgtgcagaacatctatgtgtcccacaagcctgaccacagccacgagggcgccatgttcgatgagacaatctacagcgccaccggcaagtctagaagcgccgccaaggatagaacagtgatcccctttatcgccaagaactggtcacaccccgacgaccacaacaagcagaggccttttaagggcctgatcaccgacgtgtcccagagacaccagaacaagccttacaagggcctgctgagcaacagcaactactgcatcgagatctactccgatgaggccggatggggcggacacgtgctgaaaacattcgacgcctatcagatcgtgcgggcccacaagaatgcctctgagggaatgcaggccctgcggaacaagcacagcagccagaatggccatcctctggtcatgagactgatgatcggcgactacatccgggccgagattgatggctttctgctgctgctccaagtgctgaagatcaacagcagcggcagcatcaccttcatcaagcccaacgagacaaacatcagcgcccggtatctggccaagctggctgctcagaaagcccagaaagaaggcaagcccttcgacgatatcgccctgaatgacgtgttctttcagaaggccatctccgccgatagcctgcggctgtttaaagccagacctgtgacactgagccccatcggcgaactgagagatcccggctttaaaggc

SEQ ID NO: 24 - DNA, 3210 bp, synthetic construct
  misc_feature 1..3210: note = OMNI-82 DNA optimized for expression in human cells
  source 1..3210: mol_type = other DNA
agactgggcctcgatatcggcaccaattctatcggctggtggctgtacagaaccgagaacgaccagatcacctgtgtggtggatggcggagtgcgggtgttctctgatggcagagatccccagagcaaagaaagcctggccgtggacagaagagtggctagagcccagcggcggagaagagacagatacctgagaagaaaggccgctctgatgaagagaatggccgaggctggactgatgcctgctgatcctgttcaggccaaagctctgcaggctctggacccctatgatctgagagccagaggactggatgaagccctgccactggctcatttcggcagagccctgttccacctgaaccagagaagaggcttcaagagcaaccggaaggccgacagaggcgacaatgagagcggaaagatcaaggacgccaccgccagactggactgggccatgagagatgccagagccagaacctacggcgagttcctgcacatgagacaggacaaggccgacgatcctagaagagtgcctaccgtgcggaccagactgtctgtggccagaagagataacgccgagaaagaggaagccggctacgacttctaccccgacagaaggcacctgagcgaagagtttgatgccctgtgggccgctcaggccgaacaccatacaaccctgaccgacgacctgcgggatcagatcaagaccatcatcttccaccagcggcctctgaaggctcctgaagtgggcctgtgtctgttcaccgacgagagaagaatccccagcgctcaccctctgaaccagcggagaatcctgctggaaaccgtgaacggcctgagaatcgtggcaagaggcgaagccgctagaggcctgacaagagaggaacgcgatcagatcgtgcacggcctggataacaagggccacacaaagacactgagcggcatgagcatgaagctgcgcgccatcggcaaagtgatcaagctgagatccgaccagagcttcaccctggaaacagccaacagggacgccattgcttgcgatcctgtcagagcctctctgtctcaccctgagagaatgggcggagtgtggacaacactggatgaggatgcccagtgggacgtcgtgcagagactgagagctgtgcagagcgatacagagcacgaagccctggtggattggctgatggccacacacggactcggacaggattatgcccaggccacagctaatgcccctctgcctgaaggatatggcagactgggactgaccgccaccagaaagattctggctgccctggaagccgacgtgatgagctattctgatgccgtggctgcctgtggatggtcccattctggtggacctacaggcgaagtgctggaagctctgccctactatggcgagatcctggacagacacgtgatccctggcaccggcgtgaaaaccgacgaggatgtgaagagattcggccggatcacaaaccccacagtgcacatcggactgaatcagatccggcggctggtcaacagaatcatctgtgtgcacggcaagcctgaccagatcgtggtggaagtggccagggacctgaagaacagcgaggatcagaagagagagatccagaaaaccatccggaagaacaccgacgacgccatcaagagaggcaagaagctggttgaggaactgggccagaaggacaccggcgccaatagactgattctgcggctgtgggagaacctgggcaacgatgtgatgaccagacagtgcccctacagcggcaagagaattagcgccgccatgctgttcgacggcagctgtgatgtggaccacattctgcccttcagcagaaccctggacgacagcatctggaacaagaccctgtgcctgaaagaagagaatcgcaagaaggccaacaagacaccctgggaagtgtggggcgagacagaccagtgggatgtgatcgtggccaatctgaagaacctggaccggaagcaggcttggagatttgcccctgacgccgtggaaagattcgagggcgagaacgatttcagcgctcgggccctgaaagacacccagtacctgtctagagtggctcgggcctatctggacgctctgtatgatggcgccgatggcaagtctcacgtgtgggttgtgccaggcagactgaccgaaatgctgagaaggcactggggcctgaatggcatcgaggtgctgacagactctgacgcccagaccgtgaagtccaagaacagacaggaccacagacaccacgccattgatgctgctgtggtggccgctaccgatcggagcctgatccagaggatctccaagatcgccaagcacgatgaacaggctggcgccgaacaggtggcaagatctgttcctcctccttgggacggcttcagagatgatgtggccggacagatcggcaggatcattgtgtctcacagagccgaccacggcagaatcgatcctacagccagagcacagggcagcgatacaacagccggacagctgcacatggataccgcctatggaattgtggacggcggccacgtcgtgtccagaaagcctcttatgtctctcggagccggcgacatcagaaagatcagagatcctgacctgcagcggcatctgaccagagtgacacgcggactggacaagaaagagttcgaacaggccctggcctccttcgccgcttctagaaaactgcccgatcagagcgagaacccctacttcggactgcggagagttagactgctggacgcactgcaggatagcgccagagtgcctgttagaaacgccaccggcaacatctacaaggcctataaggccggcagcaaccactgctacgaagtgtggcggatgcccgacggaaaagtgaagccttgggccatctctacctttgaggcccaccagagcggagatggctctaaacctcatcctgccgccaagcggctgctgagagtgttcaagcgcgatatggtggtcatcgagcggaagggcatcaccgtgatctgctacgtgcagaaaatggacgtggccaacggcctgtttctggtgcctcacacagagggcaacgccgacgccagaaatagggacaaagaggacgacttcaagttcatccagatgagcgccgcctctctgatcaaggccagaattagaagggtgcacgtggacgagatgggaagaatgagagatcccggacctcctcgg

SEQ ID NO: 25 - RNA, 10 nt, synthetic construct
  misc_feature 1..10: note = OMNI-59 crRNA (Repeat)
  source 1..10: mol_type = other RNA
gttccggtca

SEQ ID NO: 26 - RNA, 11 nt, synthetic construct
  misc_feature 1..11: note = OMNI-59 tracrRNA (Antirepeat)
  source 1..11: mol_type = other RNA
tggtcgctaac

SEQ ID NO: 27 - RNA, 34 nt, synthetic construct
  misc_feature 1..34: note = OMNI-59 TracrRNA Portion 1
  source 1..34: mol_type = other RNA
aagctgatgcttttgtagctagatgcaaaaaatg

SEQ ID NO: 28 - RNA, 31 nt, synthetic construct
  misc_feature 1..31: note = OMNI-59 TracrRNA Portion 2
  source 1..31: mol_type = other RNA
gaaagccgggcatgcccggctttcggctttt

SEQ ID NO: 29 - RNA, 90 nt, synthetic construct
  misc_feature 1..90: note = OMNI-59 sgRNA Scaffold V1
  source 1..90: mol_type = other RNA
gttccggtcagaaatggtcgctaacaagctgatgcttttgtagctagatgcaaaaaatggaaagccgggcatgcccggctttcggctttt

SEQ ID NO: 30 - RNA, 15 nt, synthetic construct
  misc_feature 1..15: note = OMNI-61 crRNA (Repeat)
  source 1..15: mol_type = other RNA
gttgcggccagagct

SEQ ID NO: 31 - RNA, 12 nt, synthetic construct
  misc_feature 1..12: note = OMNI-61 Partial crRNA 2
  source 1..12: mol_type = other RNA
gttgcggccaga

SEQ ID NO: 32 - RNA, 10 nt, synthetic construct
  misc_feature 1..10: note = OMNI-61 Partial crRNA 3
  source 1..10: mol_type = other RNA
gttgcggcca

SEQ ID NO: 33 - RNA, 15 nt, synthetic construct
  misc_feature 1..15: note = OMNI-61 tracrRNA (Antirepeat)
  source 1..15: mol_type = other RNA
ggctctgccgctaac

SEQ ID NO: 34 - RNA, 12 nt, synthetic construct
  misc_feature 1..12: note = OMNI-61 Partial tracrRNA 2
  source 1..12: mol_type = other RNA
tctgccgctaac

SEQ ID NO: 35 - RNA, 10 nt, synthetic construct
  misc_feature 1..10: note = OMNI-61 Partial tracrRNA 3
  source 1..10: mol_type = other RNA
tgccgctaac

SEQ ID NO: 36 - RNA, 35 nt, synthetic construct
  misc_feature 1..35: note = OMNI-61 TracrRNA Portion 1
  source 1..35: mol_type = other RNA
aaggagaaacttgttggatcaggactccacaagat

SEQ ID NO: 37 - RNA, 15 nt, synthetic construct
  misc_feature 1..15: note = OMNI-61 tracrRNA Portion 1-partial
  source 1..15: mol_type = other RNA
aaggagaaacttgtt

SEQ ID NO: 38 - RNA, 27 nt, synthetic construct
  misc_feature 1..27: note = OMNI-61 TracrRNA Portion 2
  source 1..27: mol_type = other RNA
gagacggctccctcgtggggccgtttt

SEQ ID NO: 39 - RNA, 96 nt, synthetic construct
  misc_feature 1..96: note = OMNI-61 sgRNA Scaffold V1
  source 1..96: mol_type = other RNA
gttgcggccagagctgaaaggctctgccgctaacaaggagaaacttgttggatcaggactccacaagatgagacggctccctcgtggggccgtttt

SEQ ID NO: 40 - RNA, 16 nt, synthetic construct
  misc_feature 1..16: note = OMNI-67 crRNA (Repeat)
  source 1..16: mol_type = other RNA
gctgtggttcgtcggg

SEQ ID NO: 41 - RNA, 15 nt, synthetic construct
  misc_feature 1..15: note = OMNI-67 Partial crRNA 1
  source 1..15: mol_type = other RNA
gctgtggttcgtcgg

SEQ ID NO: 42 - RNA, 12 nt, synthetic construct
  misc_feature 1..12: note = OMNI-67 Partial crRNA 2
  source 1..12: mol_type = other RNA
gctgtggttcgt

SEQ ID NO: 43 - RNA, 10 nt, synthetic construct
  misc_feature 1..10: note = OMNI-67 Partial crRNA 3
  source 1..10: mol_type = other RNA
gctgtggttc

SEQ ID NO: 44 - RNA, 16 nt, synthetic construct
  misc_feature 1..16: note = OMNI-67 tracrRNA (Antirepeat)
  source 1..16: mol_type = other RNA
cctgacttatcacagt

SEQ ID NO: 45 - RNA, 15 nt, synthetic construct
  misc_feature 1..15: note = OMNI-67 Partial tracrRNA 1
  source 1..15: mol_type = other RNA
ctgacttatcacagt

SEQ ID NO: 46 - RNA, 12 nt, synthetic construct
  misc_feature 1..12: note = OMNI-67 Partial tracrRNA 2
  source 1..12: mol_type = other RNA
acttatcacagt

SEQ ID NO: 47 - RNA, 10 nt, synthetic construct
  misc_feature 1..10: note = OMNI-67 Partial tracrRNA 3
  source 1..10: mol_type = other RNA
ttatcacagt

SEQ ID NO: 48 - RNA, 15 nt, synthetic construct
  misc_feature 1..15: note = OMNI-67 TracrRNA Portion 1
  source 1..15: mol_type = other RNA
aaggttctctaccgc

SEQ ID NO: 49 - RNA, 11 nt, synthetic construct
  misc_feature 1..11: note = OMNI-67 tracrRNA Portion 1-partial
  source 1..11: mol_type = other RNA
ggttctctacc

SEQ ID NO: 50 - RNA, 25 nt, synthetic construct
  misc_feature 1..25: note = OMNI-67 TracrRNA Portion 2
  source 1..25: mol_type = other RNA
acggcaatgtgtttacacatccgtt

SEQ ID NO: 51 - RNA, 28 nt, synthetic construct
  misc_feature 1..28: note = OMNI-67 TracrRNA Portion 3
  source 1..28: mol_type = other RNA
aaggacggtcctggaccgtccttttttt

SEQ ID NO: 52 - RNA, 104 nt, synthetic construct
  misc_feature 1..104: note = OMNI-67 sgRNA Scaffold V1
  source 1..104: mol_type = other RNA
gctgtggttcgtcggggaaacctgacttatcacagtaaggttctctaccgcacggcaatgtgtttacacatccgttaaggacggtcctggaccgtccttttttt

SEQ ID NO: 53 - RNA, 13 nt, synthetic construct
  misc_feature 1..13: note = OMNI-76 crRNA (Repeat)
  source 1..13: mol_type = other RNA
gttccggctagag

SEQ ID NO: 54 - RNA, 12 nt, synthetic construct
  misc_feature 1..12: note = OMNI-76 Partial crRNA 2
  source 1..12: mol_type = other RNA
gttccggctaga

SEQ ID NO: 55 - RNA, 10 nt, synthetic construct
  misc_feature 1..10: note = OMNI-76 Partial crRNA 3
  source 1..10: mol_type = other RNA
gttccggcta

SEQ ID NO: 56 - RNA, 14 nt, synthetic construct
  misc_feature 1..14: note = OMNI-76 tracrRNA (Antirepeat)
  source 1..14: mol_type = other RNA
ctctggacgctaac

SEQ ID NO: 57 - RNA, 13 nt, synthetic construct
  misc_feature 1..13: note = OMNI-76 Partial tracrRNA 2
  source 1..13: mol_type = other RNA
tctggacgctaac

SEQ ID NO: 58 - RNA, 11 nt, synthetic construct
  misc_feature 1..11: note = OMNI-76 Partial tracrRNA 3
  source 1..11: mol_type = other RNA
tggacgctaac

SEQ ID NO: 59 - RNA, 24 nt, synthetic construct
  misc_feature 1..24: note = OMNI-76 TracrRNA Portion 1
  source 1..24: mol_type = other RNA
aagctgaaagatgcaccaaatgat

SEQ ID NO: 60 - RNA, 12 nt, synthetic construct
  misc_feature 1..12: note = OMNI-76 tracrRNA Portion 1-partial
  source 1..12: mol_type = other RNA
gctgaaagatgc

SEQ ID NO: 61 - RNA, 25 nt, synthetic construct
  misc_feature 1..25: note = OMNI-76 TracrRNA Portion 2
  source 1..25: mol_type = other RNA
agggtcgctataggcgacccttttt

SEQ ID NO: 62 - RNA, 80 nt, synthetic construct
  misc_feature 1..80: note = OMNI-76 sgRNA Scaffold V1
  source 1..80: mol_type = other RNA
gttccggctagaggaaactctggacgctaacaagctgaaagatgcaccaaatgatagggtcgctataggcgacccttttt

SEQ ID NO: 63 - RNA, 12 nt, synthetic construct
  misc_feature 1..12: note = OMNI-79-1 crRNA (Repeat)
  source 1..12: mol_type = other RNA
gttgccgctgga

SEQ ID NO: 64 - RNA, 10 nt, synthetic construct
  misc_feature 1..10: note = OMNI-79-1 Partial crRNA 3
  source 1..10: mol_type = other RNA
gttgccgctg

SEQ ID NO: 65 - RNA, 13 nt, synthetic construct
  misc_feature 1..13: note = OMNI-79-1 tracrRNA (Antirepeat)
  source 1..13: mol_type = other RNA
tccagttgttaac

SEQ ID NO: 66 - RNA, 11 nt, synthetic construct
  misc_feature 1..11: note = OMNI-79-1 Partial tracrRNA 3
  source 1..11: mol_type = other RNA
cagttgttaac

SEQ ID NO: 67 - RNA, 22 nt, synthetic construct
  misc_feature 1..22: note = OMNI-79-1 TracrRNA Portion 1
  source 1..22: mol_type = other RNA
aagcagcttgactgcaccaaat

SEQ ID NO: 68 - RNA, 13 nt, synthetic construct
  misc_feature 1..13: note = OMNI-79-1 tracrRNA Portion 1-partial
  source 1..13: mol_type = other RNA
gcagcttgactgc

SEQ ID NO: 69 - RNA, 29 nt, synthetic construct
  misc_feature 1..29: note = OMNI-79-1 TracrRNA Portion 2
  source 1..29: mol_type = other RNA
aaggcgggggctgcggccctcgctttttt

SEQ ID NO: 70 - RNA, 80 nt, synthetic construct
  misc_feature 1..80: note = OMNI-79-1 sgRNA Scaffold V1
  source 1..80: mol_type = other RNA
gttgccgctggagaaatccagttgttaacaagcagcttgactgcaccaaataaggcgggggctgcggccctcgctttttt

SEQ ID NO: 71 - RNA, 15 nt, synthetic construct
  misc_feature 1..15: note = OMNI-79-2 crRNA (Repeat)
  source 1..15: mol_type = other RNA
gttgccgctggaccg

SEQ ID NO: 72 - RNA, 12 nt, synthetic construct
  misc_feature 1..12: note = OMNI-79-2 Partial crRNA 2
  source 1..12: mol_type = other RNA
gttgccgctgga

SEQ ID NO: 73 - RNA, 10 nt, synthetic construct
  misc_feature 1..10: note = OMNI-79-2 Partial crRNA 3
  source 1..10: mol_type = other RNA
gttgccgctg

SEQ ID NO: 74 - RNA, 16 nt, synthetic construct
  misc_feature 1..16: note = OMNI-79-2 tracrRNA (Antirepeat)
  source 1..16: mol_type = other RNA
cggtctggcagttaac

SEQ ID NO: 75 - RNA, 13 nt, synthetic construct
  misc_feature 1..13: note = OMNI-79-2 Partial tracrRNA 2
  source 1..13: mol_type = other RNA
tctggcagttaac

SEQ ID NO: 76 - RNA, 11 nt, synthetic construct
  misc_feature 1..11: note = OMNI-79-2 Partial tracrRNA 3
  source 1..11: mol_type = other RNA
tggcagttaac

SEQ ID NO: 77 - RNA, 21 nt, synthetic construct
  misc_feature 1..21: note = OMNI-79-2 TracrRNA Portion 1
  source 1..21: mol_type = other RNA
aagtgtcagtacgcaacagat

SEQ ID NO: 78 - RNA, 12 nt, synthetic construct
  misc_feature 1..12: note = OMNI-79-2 tracrRNA Portion 1-partial
  source 1..12: mol_type = other RNA
gtgtcagtacgc

SEQ ID NO: 79 - RNA, 29 nt, synthetic construct
  misc_feature 1..29: note = OMNI-79-2 TracrRNA Portion 2
  source 1..29: mol_type = other RNA
aagggcgacgctccggcgtcgcctttttt

SEQ ID NO: 80 - RNA, 85 nt, synthetic construct
  misc_feature 1..85: note = OMNI-79-2 sgRNA Scaffold V1
  source 1..85: mol_type = other RNA
gttgccgctggaccggaaacggtctggcagttaacaagtgtcagtacgcaacagataagggcgacgctccggcgtcgcctttttt

SEQ ID NO: 81 - RNA, 13 nt, synthetic construct
  misc_feature 1..13: note = OMNI-79-3 crRNA (Repeat)
  source 1..13: mol_type = other RNA
gttgccgctggac

SEQ ID NO: 82 - RNA, 12 nt, synthetic construct
  misc_feature 1..12: note = OMNI-79-3 Partial crRNA 2
  source 1..12: mol_type = other RNA
gttgccgctgga

SEQ ID NO: 83 - RNA, 10 nt, synthetic construct
  misc_feature 1..10: note = OMNI-79-3 Partial crRNA 3
  source 1..10: mol_type = other RNA
gttgccgctg

SEQ ID NO: 84 - RNA, 14 nt, synthetic construct
  misc_feature 1..14: note = OMNI-79-3 tracrRNA (Antirepeat)
  source 1..14: mol_type = other RNA
gtctggcggttaac

SEQ ID NO: 85 - RNA, 13 nt, synthetic construct
  misc_feature 1..13: note = OMNI-79-3 Partial tracrRNA 2
  source 1..13: mol_type = other RNA
tctggcggttaac

SEQ ID NO: 86 - RNA, 11 nt, synthetic construct
  misc_feature 1..11: note = OMNI-79-3 Partial tracrRNA 3
  source 1..11: mol_type = other RNA
tggcggttaac

SEQ ID NO: 87 - RNA, 22 nt, synthetic construct
  misc_feature 1..22: note = OMNI-79-3 TracrRNA Portion 1
  source 1..22: mol_type = other RNA
aagcagccagtctgcaccagat

SEQ ID NO: 88 - RNA, 13 nt, synthetic construct
  misc_feature 1..13: note = OMNI-79-3 tracrRNA Portion 1-partial
  source 1..13: mol_type = other RNA
gcagccagtctgc

SEQ ID NO: 89 - RNA, 29 nt, synthetic construct
  misc_feature 1..29: note = OMNI-79-3 TracrRNA Portion 2
  source 1..29: mol_type = other RNA
aagggcggcgctccggcgccgcctttttt

SEQ ID NO: 90 - RNA, 82 nt, synthetic construct
  misc_feature 1..82: note = OMNI-79-3 sgRNA Scaffold V1
  source 1..82: mol_type = other RNA
gttgccgctggacgaaagtctggcggttaacaagcagccagtctgcaccagataagggcggcgctccggcgccgcctttttt

SEQ ID NO: 91 - RNA, 11 nt, synthetic construct
  misc_feature 1..11: note = OMNI-80 crRNA (Repeat)
  source 1..11: mol_type = other RNA
gttgcggttgg

SEQ ID NO: 92 - RNA, 10 nt, synthetic construct
  misc_feature 1..10: note = OMNI-80 Partial crRNA 3
  source 1..10: mol_type = other RNA
gttgcggttg

SEQ ID NO: 93 - RNA, 12 nt, synthetic construct
  misc_feature 1..12: note = OMNI-80 tracrRNA (Antirepeat)
  source 1..12: mol_type = other RNA
ctggctgttaac

SEQ ID NO: 94 - RNA, 11 nt, synthetic construct
  misc_feature 1..11: note = OMNI-80 Partial tracrRNA 3
  source 1..11: mol_type = other RNA
tggctgttaac

SEQ ID NO: 95 - RNA, 22 nt, synthetic construct
  misc_feature 1..22: note = OMNI-80 TracrRNA Portion 1
  source 1..22: mol_type = other RNA
aagcagcttgactgcaccaaat

SEQ ID NO: 96 - RNA, 13 nt, synthetic construct
  misc_feature 1..13: note = OMNI-80 tracrRNA Portion 1-partial
  source 1..13: mol_type = other RNA
gcagcttgactgc

SEQ ID NO: 97 - RNA, 27 nt, synthetic construct
  misc_feature 1..27: note = OMNI-80 TracrRNA Portion 2
  source 1..27: mol_type = other RNA
aagggcagggctgcggccctgcctttt

SEQ ID NO: 98 - RNA, 76 nt, synthetic construct
  misc_feature 1..76: note = OMNI-80 sgRNA Scaffold V1
  source 1..76: mol_type = other RNA
gttgcggttgggaaactggctgttaacaagcagcttgactgcaccaaataagggcagggctgcggccctgcctttt

SEQ ID NO: 99 - RNA, 13 nt, synthetic construct
  misc_feature 1..13: note = OMNI-81 crRNA (Repeat)
  source 1..13: mol_type = other RNA
gttccggctagag

SEQ ID NO: 100 - RNA, 12 nt, synthetic construct
  misc_feature 1..12: note = OMNI-81 Partial crRNA 2
  source 1..12: mol_type = other RNA
gttccggctaga

SEQ ID NO: 101 - RNA, 10 nt, synthetic construct
  misc_feature 1..10: note = OMNI-81 Partial crRNA 3
  source 1..10: mol_type = other RNA
gttccggcta

SEQ ID NO: 102 - RNA, 14 nt, synthetic construct
  misc_feature 1..14: note = OMNI-81 tracrRNA (Antirepeat)
  source 1..14: mol_type = other RNA
ctctagacgctaac

SEQ ID NO: 103 - RNA, 13 nt, synthetic construct
  misc_feature 1..13: note = OMNI-81 Partial tracrRNA 2
  source 1..13: mol_type = other RNA
tctagacgctaac

SEQ ID NO: 104 - RNA, 11 nt, synthetic construct
  misc_feature 1..11: note = OMNI-81 Partial tracrRNA 3
  source 1..11: mol_type = other RNA
tagacgctaac

SEQ ID NO: 105 - RNA, 22 nt, synthetic construct
  misc_feature 1..22: note = OMNI-81 TracrRNA Portion 1
  source 1..22: mol_type = other RNA
aagctgaaagatgcaccaaatg

SEQ ID NO: 106 - RNA, 12 nt, synthetic construct
  misc_feature 1..12: note = OMNI-81 tracrRNA Portion 1-partial
  source 1..12: mol_type = other RNA
gctgaaagatgc

SEQ ID NO: 107 - RNA, 30 nt, synthetic construct
  misc_feature 1..30: note = OMNI-81 TracrRNA Portion 2
  source 1..30: mol_type = other RNA
gaaagccgctatatgcggctttcgtctttt

SEQ ID NO: 108 - RNA, 83 nt, synthetic construct
  misc_feature 1..83: note = OMNI-81 sgRNA Scaffold V1
  source 1..83: mol_type = other RNA
gttccggctagaggaaactctagacgctaacaagctgaaagatgcaccaaatggaaagccgctatatgcggctttcgtctttt

SEQ ID NO: 109 - RNA, 16 nt, synthetic construct
  misc_feature 1..16: note = OMNI-82 crRNA (Repeat)
  source 1..16: mol_type = other RNA
gttgcggctggaccgc

SEQ ID NO: 110 - RNA, 15 nt, synthetic construct
  misc_feature 1..15: note = OMNI-82 Partial crRNA 1
  source 1..15: mol_type = other RNA
gttgcggctggaccg

SEQ ID NO: 111 - RNA, 12 nt, synthetic construct
  misc_feature 1..12: note = OMNI-82 Partial crRNA 2
  source 1..12: mol_type = other RNA
gttgcggctgga

SEQ ID NO: 112 - RNA, 10 nt, synthetic construct
  misc_feature 1..10: note = OMNI-82 Partial crRNA 3
  source 1..10: mol_type = other RNA
gttgcggctg

SEQ ID NO: 113 - RNA, 17 nt, synthetic construct
  misc_feature 1..17: note = OMNI-82 tracrRNA (Antirepeat)
  source 1..17: mol_type = other RNA
gcggtcgagctgttaac

SEQ ID NO: 114 - RNA, 16 nt, synthetic construct
  misc_feature 1..16: note = OMNI-82 Partial tracrRNA 1
  source 1..16: mol_type = other RNA
cggtcgagctgttaac

SEQ ID NO: 115 - RNA, 13 nt, synthetic construct
  misc_feature 1..13: note = OMNI-82 Partial tracrRNA 2
  source 1..13: mol_type = other RNA
tcgagctgttaac

SEQ ID NO: 116 - RNA, 11 nt, synthetic construct
  misc_feature 1..11: note = OMNI-82 Partial tracrRNA 3
  source 1..11: mol_type = other RNA
gagctgttaac

SEQ ID NO: 117 - RNA, 22 nt, synthetic construct
  misc_feature 1..22: note = OMNI-82 TracrRNA Portion 1
  source 1..22: mol_type = other RNA
aagcattcgattgcaccacatt

SEQ ID NO: 118 - RNA, 12 nt, synthetic construct
  misc_feature 1..12: note = OMNI-82 tracrRNA Portion 1-partial
  source 1..12: mol_type = other RNA
gcattcgattgc

SEQ ID NO: 119 - RNA, 28 nt, synthetic construct
  misc_feature 1..28: note = OMNI-82 TracrRNA Portion 2
  source 1..28: mol_type = other RNA
gaagcgcagggccacggccctgcgtttt

SEQ ID NO: 120 - RNA, 87 nt, synthetic construct
  misc_feature 1..87: note = OMNI-82 sgRNA Scaffold V1
  source 1..87: mol_type = other RNA
gttgcggctggaccgcgaaagcggtcgagctgttaacaagcattcgattgcaccacattgaagcgcagggccacggccctgcgtttt

SEQ ID NO: 121 - DNA, 6616 bp, synthetic construct
  misc_feature 1..6616: note = pbNNC-3
  source 1..6616: mol_type = other DNA
gttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcgggatctcgacgctaaattaatacgactcactataggggaattgtgagcggataacaattcccctgtagaaataattttgtttaactaaagaggagaaatttcatatgtacccatacgatgtgccagattacgctggcaccgagctcggtaccgagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggatggatccccaaaaaagaaaagaaaagttgctgccgcactcgagcatcatcaccatcaccatcatcattaagcggccgcctagcataaccccttggggcctctaaacgggtcttgaggggttttttgacctaggctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcctcgagtcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgacacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagtt

SEQ ID NO: 122 - DNA, 2549 bp, synthetic construct
  misc_feature 1..2549: note = Artificial Sequence
  source 1..2549: mol_type = other DNA
gcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaacacatttccccgaaaagtgccacctgacgtcctcgagtcccgcataatcgaaattaatacgactcactatagggaagagcagagccttggtctcgttgccgctggagaaatccagttgttaacaagcagcttgactgcaccaaataaggcgggggctgcggccctcgcttttttgaattcttccgctgagcaataactagcataaccccttggggcctctaaacgggtcttgaggggttttttgacctaggctaggggatatattccgcttcctcgcggtaccccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccg

SEQ ID NO: 123 - DNA, 5009 bp, synthetic construct
  misc_feature 1..5009: note = pbPOS T2 library
  misc_feature 3040..3047: note = n is a, c, g, or t
  source 1..5009: mol_type = other DNA
tcgagtctttacactttatgcttccggctcgtatgttgtgtggaattgtgagcggataacaatttcacacatgattacggattcaacgtcgtgactggtaaaacccgggcgttacccaacttaatcgccttgcagcacatccccctttcgccagcaggcgtaataaggaaaggattcatgtactatttgaaaaacacaaacttttggatgttcggtttattctttttcttttacttttttatcatgggagcctacttcccgtttttcccgatttggctacatgatatcaaccatatcagcaaaagtgatacgggtattatttttgccgctatttctctgttctcgctattattccaaccgctgtttggtctgctttctgacaaactcggtctacgcaaatacctgctgtggattattaccggcatgttagtgatgtttgcgccgttctttatttttatcttcgggccactgctgcagtacaacattttagtagggtcgattgttggtggtatttatctaggctttagttttaacgccggtgcgccagcagtagaggcatttattgagaaagtcagccggcgcagtaatttcgaatttggtcgcgcgcggatgtttggcagtgttggctgggcgctggttgcctcgattgtcgggatcatgttcaccattaataatcagtttgttttctggctgggctctggcagttgtctcatcctcgccgttttactctttttcgccaaaacggacgcgccctcaagtgccacggttgccaatgcggtaggtgccaaccattcggcatttagccttaagctggcactggaactgttcagacagccaaaactgtggtttttgtcactgtatgttattggcgtttcctccacctacgatgtttttgaccaacagtttgctaatttctttacttcgttctttgctaccggtgaacagggtacccgcgtatttggctacgtaacgacaatgggcgaattacttaacgcctcgattatgttctttgcgccactgatcattaatcgcatcggtgggaagaatgccctgctgctggctggcactattatgtctgtacgtattattggctcatcgttcgccacctcagcgctggaagtggttattctgaaaacgctgcatatgtttgaagtaccgttcctgctggtgggctcctttaaatatattactagtcagtttgaagtgcgtttttcagcgacgatttatctggtcagtttcagcttctttaagcaactggcgatgatttttatgtctgtactggcgggcaatatgtatgaaagcataggtttccaaggcgcttatctggtgctgggtctggtggcgctgggcttcaccttaatttccgtgttcacgcttagcggcccgggcccgctttccctgctgcgtcgtcaggtgaatgaagtcgcttaaaggcctcgatgcagctagcatgctaatctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggggtagcgattgaaaacgatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttttggggaatataatcttctagacatacaatggaagagcagagccttggtctcnnnnnnnnaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacc

SEQ ID NO: 124 - DNA, 5515 bp, synthetic construct
  misc_feature 1..5515: note = pET9a
  source 1..5515: mol_type = other DNA
taatacgactcactatagggagaccacaacggtttccctctagagagacaataaccctgataatgcttcaataatattgaaaaaggaagagtatgcctaagaagaagagaaaggtgggtaccgagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggatggatcctacccatacgatgttccagattacgcggccgctccaaaaaagaaaagaaaagttgcggctagccatcatcaccatcaccatcatcattaaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcgatcccgcgaaat

SEQ ID NO: 125 - DNA, 9428 bp, synthetic construct
  misc_feature 1..9428: note = pmOMNI
  source 1..9428: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgcctaagaagaagagaaaggtgggtaccgagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggatggatcctacccatacgatgttccagattacgcggccgctccaaaaaagaaaagaaaagttgaattcggcggcagcggcgccaccaacttcagcctgctgaagcaggccggcgacgtggaggagaaccccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 126 - DNA, 2726 bp, synthetic construct
  misc_feature 1..2726: note = pmGuide T2/Endogenic site
  source 1..2726: mol_type = other DNA
ggaagagcagagccttggtctcgttccggctagaggaaactctggacgctaacaagctgaaagatgcaccaaatgatagggtcgctataggcgaccctttttgaattcttccgctgagcaataactagcataaccccttggggcctctaaacgggtcttgaggggttttttgacctaggctaggggatatattccgcttcctcgcggtaccccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 127 - DNA, 4194 bp, synthetic construct
  misc_feature 1..4194: note = pPML3.1
  misc_feature 2256..2261: note = n is a, c, g, or t
  source 1..4194: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattctgcagatatcaaagcttgccaccatgcatacaatggaagagcagagccttggtctcnnnnnngcgggtctggtggcgctagcgtgtccaagggcgaggagctgttcaccggcgtggtgcccatcctggtggagctggacggcgacgtgaacggccacaagttcagcgtgagcggcgagggcgaaggggacgctacttacggcaaactgactctcaagtttatctgtactaccgggaagctccctgtcccttggcctacactggtcacaactctcacatatggggtccagtgcttcagcagataccccgaccacatgaagcagcacgacttcttcaagagcgccatgcccgagggctacgtgcaggagagaaccatcttcttcaaggacgacggcaactacaagaccagagctgaggtcaagtttgagggtgacaccctggtgaacagaatcgagctgaagggcatcgacttcaaggaggacggcaacatcctgggccacaagctggagtacaactacaacagccacaacgtgtacatcatggctgataaacagaagaatgggattaaggtgaacttcaagatcagacacaacatcgaggacggcagcgtgcagctggccgaccactaccagcagaacacccccatcggcgacggccccgtgctgctgcccgacaaccactacctgagcacccagagcgctctcagtaaggaccctaatgagaagagagaccacatggtgctgctggagttcgtgaccgccgccggcatcaccctgggcatggacgagctgtacaagtgagggcctaatgagtttggaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 128 - DNA, 4566 bp, synthetic construct
  misc_feature 1..4566: note = AAV
  source 1..4566: mol_type = other DNA
tctagacaactttgtatagaaaagttggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggaccaggtgaccaccgtgagttgccgctggagaaatccagttgttaacaagcagcttgactgcaccaaataaggcgggggctgcggccctcgctttttcaagtttgtacaaaaaagcaggcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcacccagctttcttgtacaaagtgggccaccatggcccctaagaagaagagaaaggtgggtaccgagaagagactgggcctcgacatcggcacaaatagcatcggctggtgcctgtacgagggcgactctatcctggatatcggcgtgcggatcttcagcgacggcagagatcctaaatctggcgcctctctggccgtggacagacggaatgctagagccatgcggcggagaagggatagatacctgggaagaagaagcgccctgatcaaggccctgaaggcccatggactgtttccagccgaacaggatgccgccaaggctctggaaagagaggacccttacagcctgagagtcagagccctggatcacagactggaccctcatcagatcggcagagccatcttccacctgaaccagcggagaggcttcagaagcaacagaaaggccgacagagtgctgggcgatcaagagagcggactgatcagcacagccaccagagttctggacgaagccatggccaaaagcggcgctagaacactgggagagtttctggccagccgggacactagaagagtgcggatgaggcctgacgtgaagggctacgacttctaccccaaccggcagcactacctggaagagttcgagaagatctgggacgcccagagccagtatcaccctgatctgctgtctcagcaggccaagagcgccatccaccggatcatttttcaccagaggccactgaagccacaggccgtgggaacatgtacatttgccggactgcatggcatccccggcgacgagacaagactgcctaaagctcaccctctgttccagcagcggcggctgtatgaggaagtgaaccagctggaaatcgtgtgcgccagcgctcctgccagaaagctgaccagagatgagagagatgccctgatcctgaagctgcaggacaagaaaaaagtgaccttcagcacactggcccggaccatcagactgaaagagggcgagagattcaacaaagagagcgagaaccggaaggacctggctggggatgaagtgcgggccgagatgagcgataagaccagattcggcagacggtggtttcacctgagcctggatgagcagtggtccgtgatcgacagactgctgaacgaggaaagcaccgaggatctgctggcctggctggaaaaagagtggtccctgccttctgatgtggccgaagccgtggctaatgcccatctgcctgatggccacggaagatttggcctgacagccaccgttagactgctcgaacacctgaaagccgacgtggtcacatatgccgaagctgctcggagagccggcttccaccactctgatttcagagatggcgcctgctacgacgagctgccttactatggcgagatcctgagcagagagatcgcccctggaaaggacgagtacggcgatccactggaaagacagtggggcaagatcacaaaccccaccgtgcacatcggcctgaatcagctgagaaggctgattaacgccctcgtgcggagacacggcagacccgatttcatcttcgtggaactggcccgcgagctgaagctgaatgagaagcagaaggccgaccacaagcggcggatcaagcagacaacagatgccgctagagctagagccgagaagctgagagaaatcggacagagagacagcggctccaaccggatgctgctgcggatttgggaagaactgaaccccagcaatcccctggacagaagatgcccttattgcgccgagcctatctccatcgagatgctgatgagcggcagcgccgacatcgatcacatcgtgccttacagcagatgcctggacgatagcgccgccaacaaagtggtggcccacaaccactgcaacagacagaagggcaacagaaccccttgggagcagtggggacagaccacaagatggcccctgattcaagaacaggtggcccggatgcacagatccaaacaatggcgcttcggccccgacgccatggaacgagttgatcgagatggcggctttatcgcccggcagctgacagatacccagtacctgtctagaatcgccgcacagtacctgagcgccctgtacacacctgatgaaggcagacgggtgtacgccgtgacaggcagactgactgctatgcttagacggctgtggggcctgaacgacatcctgcctgatcacaactgggtgctgaaccctcacagcaacgcccctaagaacaggctggatcatagacaccacgccatcgatgccgccgttgtgggagctacaacacccgccatgattcagcaggttgccagagctgctgcaagagccgaggaacaggacctggatagactgttcgctgacctgcctcctccatggccaggctttagagaggaactgcagggcagaattatggccgctgtggtgtctcacaagcccgaccatggaagaaagggcagaccactgcctggcagagatagcacatccggcagactgcacaatgacaccgcctacggctttactggccgcagaaatgccaagggcatgcctatcgtggtcaccagaaaacccctgctggccctgaaacctgaggacctgacagaccccgagagaatcccagatcctgctctgcagggcgcactgtttgaagccactagaggcgctaccggcaaggacttcgagaaagccctgcgggacttcagcagaagagatggcccttaccagggcatcagacggattagactgaccgaggctctgaacgtgatccccatcagagatagaaccggccacgcctacaaaggcgtgaaaggcgacgccaacgccagattcgatgtttggagactgcccgacggcaagtggatcaccagatggaaggatagagatggcatcgagcacagcggcatcgtgtccctgtttgatgctcaccagcctagccaggtgtaccacagacctcatcctgccgccaaaaaggtgctgagcctgagacagaatgacctggtggcagtggaacacgatggcgaccccggaaagatcatgcgggtcgtgaagtttagcgccaacggcagcatcacattcgccccacataatgaggccggacctctgaaaacccgggacaccgatcctgccgatcctttcagatacgtgacaacagtggccagcggcctgaagaagatgagagccagacaagtgcggatcgatgagctgggcaaagtgcacgatcccggacctagagaggatggatcctacccatacgatgttccagattacgcggccgctccaaaaaagaaaagaaaagtttgacaactttattatacatagttggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgc

SEQ ID NO: 129 - protein, 9 aa, synthetic construct
  REGION 1..9: note = HA-tag
YPYDVPDYA

SEQ ID NO: 130 - protein, 7 aa, synthetic construct
  REGION 1..7: note = NLS
PKKKRKV

SEQ ID NO: 131 - protein, 19 aa, synthetic construct
  REGION 1..19: note = P2A
ATNFSLLKQAGDVEENPGP

SEQ ID NO: 132 - protein, 236 aa, synthetic construct
  REGION 1..236: note = mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 133 - DNA, 27 bp, synthetic construct
  misc_feature 1..27: note = HA-tag
  source 1..27: mol_type = other DNA
tacccatacgatgttccagattacgct

SEQ ID NO: 134 - DNA, 21 bp, synthetic construct
  misc_feature 1..21: note = NLS
  source 1..21: mol_type = other DNA
ccaaaaaagaaaagaaaagtt

SEQ ID NO: 135 - DNA, 57 bp, synthetic construct
  misc_feature 1..57: note = P2A
  source 1..57: mol_type = other DNA
gccaccaacttcagcctgctgaagcaggccggcgacgtggaggagaaccccggcccc

SEQ ID NO: 136 - DNA, 711 bp, synthetic construct
  misc_feature 1..711: note = mCherry
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 137 - DNA, 22 bp, synthetic construct
  misc_feature 1..22: note = OMNI59_CXCR4_S1
  source 1..22: mol_type = other DNA
agcgcccgcttgggggaggagg

SEQ ID NO: 138 - DNA, 22 bp, synthetic construct
  misc_feature 1..22: note = OMNI59_CXCR4_S2
  source 1..22: mol_type = other DNA
caatataccccaagcaccgaag

SEQ ID NO: 139 - DNA, 22 bp, synthetic construct
  misc_feature 1..22: note = OMNI59_PDCD1_S1
  source 1..22: mol_type = other DNA
taagaaccatcctggccgccag

SEQ ID NO: 140 - DNA, 22 bp, synthetic construct
  misc_feature 1..22: note = OMNI59_PDCD1_S2
  source 1..22: mol_type = other DNA
gtaccagtttagcacgaagctc

SEQ ID NO: 141 - DNA, 22 bp, synthetic construct
  misc_feature 1..22: note = OMNI59_TRAC_S1
  source 1..22: mol_type = other DNA
ctggaataatgctgttgttgaa

SEQ ID NO: 142 - DNA, 22 bp, synthetic construct
  misc_feature 1..22: note = OMNI59_TRAC_S2
  source 1..22: mol_type = other DNA
tgatgtcaagctggtcgagaaa

SEQ ID NO: 143 - DNA, 22 bp, synthetic construct
  misc_feature 1..22: note = OMNI67_CXCR4_S3
  source 1..22: mol_type = other DNA
cgccaagtgataaacacgagga

SEQ ID NO: 144 - DNA, 22 bp, synthetic construct
  misc_feature 1..22: note = OMNI67_CXCR4_S4
  source 1..22: mol_type = other DNA
ttgggcgggagtgtcagaaaat

SEQ ID NO: 145 - DNA, 22 bp, synthetic construct
  misc_feature 1..22: note = OMNI67_PDCD1_S3
  source 1..22: mol_type = other DNA
atcctggccgccagcccagttg

SEQ ID NO: 146 - DNA, 22 bp, synthetic construct
  misc_feature 1..22: note = OMNI67_PDCD1_S4
  source 1..22: mol_type = other DNA
taaactggtaccgcatgagccc

SEQ ID NO: 147 - DNA, 22 bp, synthetic construct
  misc_feature 1..22: note = OMNI67_TRAC_S3
  source 1..22: mol_type = other DNA
agctgagagactctaaatccag

SEQ ID NO: 148 - DNA, 22 bp, synthetic construct
  misc_feature 1..22: note = OMNI67_TRAC_S4
  source 1..22: mol_type = other DNA
gacttcaagagcaacagtgctg

SEQ ID NO: 149 - DNA, 22 bp, synthetic construct
  misc_feature 1..22: note = OMNI79_CXCR4_S25
  source 1..22: mol_type = other DNA
tggcaagagacccacacaccgg

SEQ ID NO: 150 - DNA, 22 bp, synthetic construct
  misc_feature 1..22: note = OMNI79_CXCR4_S26
  source 1..22: mol_type = other DNA
acacaccggaggagcgcccgct

SEQ ID NO: 151 - DNA, 22 bp, synthetic construct
  misc_feature 1..22: note = OMNI79_g58_Ref
  source 1..22: mol_type = other DNA
cagctgcgggaaagggattccc

SEQ ID NO: 152 - DNA, 22 bp, synthetic construct
  misc_feature 1..22: note = OMNI79_g35
  source 1..22: mol_type = other DNA
gcagtccgggctgggagcgggt

SEQ ID NO: 153 - DNA, 22 bp, synthetic construct
  misc_feature 1..22: note = OMNI81_CXCR4_S6
  source 1..22: mol_type = other DNA
gggtatattgggcgggagtgtc

SEQ ID NO: 154 - DNA, 22 bp, synthetic construct
  misc_feature 1..22: note = OMNI81_CXCR4_S7
  source 1..22: mol_type = other DNA
ccactacaagttgcttgaagcc

SEQ ID NO: 155 - DNA, 22 bp, synthetic construct
  misc_feature 1..22: note = OMNI81_PDCD1_S7
  source 1..22: mol_type = other DNA
ggtcaccacgagcagggctggg

SEQ ID NO: 156 - DNA, 22 bp, synthetic construct
  misc_feature 1..22: note = OMNI81_PDCD1_S8
  source 1..22: mol_type = other DNA
cacgaagctctccgatgtgttg

SEQ ID NO: 157 - DNA, 22 bp, synthetic construct
  misc_feature 1..22: note = OMNI81_TRAC_S6
  source 1..22: mol_type = other DNA
ttactttgtgacacatttgttt

SEQ ID NO: 158 - DNA, 22 bp, synthetic construct
  misc_feature 1..22: note = OMNI81_TRAC_S7
  source 1..22: mol_type = other DNA
ggctggggaagaaggtgtcttc

SEQ ID NO: 159 - DNA, 331 bp, synthetic construct
  misc_feature 1..331: note = OMNI59_CXCR4_S1 3' (PAM containing) genomic seq
  source 1..331: mol_type = other DNA
gccaagtgataaacacgaggatggcaagagacccacacaccggaggagcgcccgcttgggggaggaggtgccgtttgttcattttctgacactcccgcccaatataccccaagcaccgaagggccttcgttttaagaccgcattctctttacccactacaagttgcttgaagcccagaatggtttgtatttaggcaggcgtgggaaaattaagtttttgcgctttaggagaatgagtctttgcaacgcccccgccctccccccgtgatcctcccttctcccctcttccctccctgggcgaaaaacttcttacaaaaagttaatcactgccc

SEQ ID NO: 160 - DNA, 331 bp, synthetic construct; identical to SEQ ID NO: 159
  misc_feature 1..331: note = OMNI59_CXCR4_S2 3' (PAM containing) genomic seq
  source 1..331: mol_type = other DNA

SEQ ID NO: 161 - DNA, 370 bp, synthetic construct
  misc_feature 1..370: note = OMNI59_PDCD1_S1 3' (PAM containing) genomic seq
  source 1..370: mol_type = other DNA
gctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccc

SEQ ID NO: 162 - DNA, 494 bp, synthetic construct
  misc_feature 1..494: note = OMNI59_PDCD1_S2 3' (PAM containing) genomic seq
  source 1..494: mol_type = other DNA
ccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctgg

SEQ ID NO: 163 - DNA, 429 bp, synthetic construct
  misc_feature 1..429: note = OMNI59_TRAC_S1 3' (PAM containing) genomic seq
  source 1..429: mol_type = other DNA
gtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcc

SEQ ID NO: 164 - DNA, 447 bp, synthetic construct
  misc_feature 1..447: note = OMNI59_TRAC_S2 3' (PAM containing) genomic seq
  source 1..447: mol_type = other DNA
gcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaac

SEQ ID NO: 165 - DNA, 421 bp, synthetic construct
  misc_feature 1..421: note = OMNI67_CXCR4_S3 3' (PAM containing) genomic seq
  source 1..421: mol_type = other DNA
ggaaatgaaacttggggcgaggaccacgggtgcagaccccgttaccttctccacccaggaaaatgccccgctccctaacgtcccaaacgcgccaagtgataaacacgaggatggcaagagacccacacaccggaggagcgcccgcttgggggaggaggtgccgtttgttcattttctgacactcccgcccaatataccccaagcaccgaagggccttcgttttaagaccgcattctctttacccactacaagttgcttgaagcccagaatggtttgtatttaggcaggcgtgggaaaattaagtttttgcgctttaggagaatgagtctttgcaacgcccccgccctccccccgtgatcctcccttctcccctcttccctccctgggcgaaaaacttcttacaaaaagttaatcactgccc

SEQ ID NO: 166 - DNA, 439 bp, synthetic construct
  misc_feature 1..439: note = OMNI67_CXCR4_S4 3' (PAM containing) genomic seq
  source 1..439: mol_type = other DNA
gatggcaagagacccacacaccggaggagcgcccgcttgggggaggaggtgccgtttgttcattttctgacactcccgcccaatataccccaagcaccgaagggccttcgttttaagaccgcattctctttacccactacaagttgcttgaagcccagaatggtttgtatttaggcaggcgtgggaaaattaagtttttgcgctttaggagaatgagtctttgcaacgcccccgccctccccccgtgatcctcccttctcccctcttccctccctgggcgaaaaacttcttacaaaaagttaatcactgcccctcctagcagcacccaccccaccccccacgccgcctgggagtggcctctttgtgtgtattttttttttcctcctaaggaaggttttttttcttccctctagtgggcggggcagaggagttagccaag

SEQ ID NO: 167 - DNA, 386 bp, synthetic construct
  misc_feature 1..386: note = OMNI67_PDCD1_S3 3' (PAM containing) genomic seq
  source 1..386: mol_type = other DNA
gctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgg

SEQ ID NO: 168 - DNA, 494 bp, synthetic construct; identical to SEQ ID NO: 162
  misc_feature 1..494: note = OMNI67_PDCD1_S4 3' (PAM containing) genomic seq
  source 1..494: mol_type = other DNA

SEQ ID NO: 169 - DNA, 463 bp, synthetic construct
  misc_feature 1..463: note = OMNI67_TRAC_S3 3' (PAM containing) genomic seq
  source 1..463: mol_type = other DNA
cctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccac

SEQ ID NO: 170 - DNA, 463 bp, synthetic construct; identical to SEQ ID NO: 169
  misc_feature 1..463: note = OMNI67_TRAC_S4 3' (PAM containing) genomic seq
  source 1..463: mol_type = other DNA

SEQ ID NO: 171 - DNA, 421 bp, synthetic construct; identical to SEQ ID NO: 165
  misc_feature 1..421: note = OMNI79_CXCR4_S25 3' (PAM containing) genomic seq
  source 1..421: mol_type = other DNA

SEQ ID NO: 172 - DNA, 382 bp, synthetic construct
  misc_feature 1..382: note = OMNI79_CXCR4_S26 3' (PAM containing) genomic seq
  source 1..382: mol_type = other DNA
cgttaccttctccacccaggaaaatgccccgctccctaacgtcccaaacgcgccaagtgataaacacgaggatggcaagagacccacacaccggaggagcgcccgcttgggggaggaggtgccgtttgttcattttctgacactcccgcccaatataccccaagcaccgaagggccttcgttttaagaccgcattctctttacccactacaagttgcttgaagcccagaatggtttgtatttaggcaggcgtgggaaaattaagtttttgcgctttaggagaatgagtctttgcaacgcccccgccctccccccgtgatcctcccttctcccctcttccctccctgggcgaaaaacttcttacaaaaagttaatcactgccc

SEQ ID NO: 173 - DNA, 350 bp, synthetic construct
  misc_feature 1..350: note = OMNI79_g58_Ref 3' (PAM containing) genomic seq
  source 1..350: mol_type = other DNA
agggccattgtctccctaacccgagagccatgggggtccacttgcctgtggtcacgtcaggactccagcctggcccaggctctgcgtgtccccgggtgccctcgccccgcctattcctggagacaggcccgttggttcccttcccctccccttgtcctggagccaggaggacgttggttcttgcgacagccttggcccggccgttgcagctggaacatcgtgggggagatgggaagaggaacggggcccggagcccggggctgggtcctgggaatccctttcccgcagctgggactccagctcccctgccagttcctccaggcggaagccctcaggcttggtcctcactc

SEQ ID NO: 174 - DNA, 443 bp, synthetic construct
  misc_feature 1..443: note = OMNI79_g35 3' (PAM containing) genomic seq
  source 1..443: mol_type = other DNA
ggcgtctgtttcgtacgtgccctgggtgtccctctgctccccacccgctcccagcccggactgcagcaacaggcaccgtggctagaccctaggagggacttcccaaccctgacaggcggcgggcaggtgggcagggcctcgcagtccagcttccccaccttgtctgcctccacagggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaata

SEQ ID NO: 175 - DNA, 439 bp, synthetic construct; identical to SEQ ID NO: 166
  misc_feature 1..439: note = OMNI81_CXCR4_S6 3' (PAM containing) genomic seq
  source 1..439: mol_type = other DNA

SEQ ID NO: 176 - DNA, 422 bp, synthetic construct
  misc_feature 1..422: note = OMNI81_CXCR4_S7 3' (PAM containing) genomic seq
  source 1..422: mol_type = other DNA
ggaggaggtgccgtttgttcattttctgacactcccgcccaatataccccaagcaccgaagggccttcgttttaagaccgcattctctttacccactacaagttgcttgaagcccagaatggtttgtatttaggcaggcgtgggaaaattaagtttttgcgctttaggagaatgagtctttgcaacgcccccgccctccccccgtgatcctcccttctcccctcttccctccctgggcgaaaaacttcttacaaaaagttaatcactgcccctcctagcagcacccaccccaccccccacgccgcctgggagtggcctctttgtgtgtattttttttttcctcctaaggaaggttttttttcttccctctagtgggcggggcagaggagttagccaagatgtgactttgaaaccctcagcgt

SEQ ID NO: 177 - DNA, 435 bp, synthetic construct
  misc_feature 1..435: note = OMNI81_PDCD1_S7 3' (PAM containing) genomic seq
  source 1..435: mol_type = other DNA
cccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctg

SEQ ID NO: 178 - DNA, 494 bp, synthetic construct; identical to SEQ ID NO: 162
  misc_feature 1..494: note = OMNI81_PDCD1_S8 3' (PAM containing) genomic seq
  source 1..494: mol_type = other DNA

SEQ ID NO: 179 - DNA, 463 bp, synthetic construct; identical to SEQ ID NO: 169
  misc_feature 1..463: note = OMNI81_TRAC_S6 3' (PAM containing) genomic seq
  source 1..463: mol_type = other DNA

SEQ ID NO: 180 - DNA, 429 bp, synthetic construct; identical to SEQ ID NO: 163
  misc_feature 1..429: note = OMNI81_TRAC_S7 3' (PAM containing) genomic seq
  source 1..429: mol_type = other DNA

SEQ ID NO: 181 - DNA, 22 bp, synthetic construct
  misc_feature 1..22: note = OMNI79_PDCD1_S24
  source 1..22: mol_type = other DNA
gtcgtctgggcggtgctacaac

SEQ ID NO: 182 - DNA, 22 bp, synthetic construct
  misc_feature 1..22: note = OMNI79_PDCD1_S25
  source 1..22: mol_type = other DNA
tgctacaactgggctggcggcc

SEQ ID NO: 183 - DNA, 22 bp, synthetic construct
  misc_feature 1..22: note = OMNI79_TRAC_S21
  source 1..22: mol_type = other DNA
actgtgctagacatgaggtcta

SEQ ID NO: 184 - DNA, 22 bp, synthetic construct
  misc_feature 1..22: note = OMNI79_TRAC_S24
  source 1..22: mol_type = other DNA
ggccactttcaggaggaggatt

SEQ ID NO: 185 - DNA, 22 bp, synthetic construct
  misc_feature 1..22: note = OMNI79_EMX_S2
  source 1..22: mol_type = other DNA
cttctgtgaatgttagacccat

SEQ ID NO: 186 - DNA, 22 bp, synthetic construct
  misc_feature 1..22: note = OMNI79_EMX_S3
  source 1..22: mol_type = other DNA
acccatgggagcagctggtcag

SEQ ID NO: 187 - DNA, 22 bp, synthetic construct
  misc_feature 1..22: note = OMNI79_SAMD9_rs070_1
  source 1..22: mol_type = other DNA
ttagagtagaaatcgtctcatt

SEQ ID NO: 188 - DNA, 22 bp, synthetic construct
  misc_feature 1..22: note = OMNI79_SAMD9_rs070_2
  source 1..22: mol_type = other DNA
gtagaaatcgtctcatttggtg

SEQ ID NO: 189 - DNA, 22 bp, synthetic construct
  misc_feature 1..22: note = OMNI79_SAMD9_rs201_alt
  source 1..22: mol_type = other DNA
tttaatctggaagtaatcatat

SEQ ID NO: 190 - DNA, 22 bp, synthetic construct
  misc_feature 1..22: note = OMNI79_SAMD9_rs499_1
  source 1..22: mol_type = other DNA
ggatctcttgggtcatccacag

SEQ ID NO: 191 - DNA, 22 bp, synthetic construct
  misc_feature 1..22: note = OMNI79_SAMD9_rs499_2
  source 1..22: mol_type = other DNA
gtatttctattactgtcctctg

SEQ ID NO: 192 - DNA, 22 bp, synthetic construct
  misc_feature 1..22: note = OMNI79_SAMD9L_rs532_1
  source 1..22: mol_type = other DNA
ctttttgatcattacattgaag

SEQ ID NO: 193 - DNA, 22 bp, synthetic construct
  misc_feature 1..22: note = OMNI79_SAMD9L_rs532_2
  source 1..22: mol_type = other DNA
attacattgaagtggtcaatga

SEQ ID NO: 194 - DNA, 22 bp, synthetic construct
  misc_feature 1..22: note = OMNI79_Serpina_S12
  source 1..22: mol_type = other DNA
gtggaccaggtgaccaccgtga

SEQ ID NO: 195 - DNA, 22 bp, synthetic construct
  misc_feature 1..22: note = OMNI79_Serpina_S13
  source 1..22: mol_type = other DNA
cacggtggtcacctggtccacg

SEQ ID NO: 196 - DNA, 22 bp, synthetic construct
  misc_feature 1..22: note = OMNI79_Serpina_S14
  source 1..22: mol_type = other DNA
aggcaccttcacggtggtcacc

SEQ ID NO: 197 - DNA, 22 bp, synthetic construct
  misc_feature 1..22: note = OMNI79_Serpina_S22
  source 1..22: mol_type = other DNA
gactccaaaacctgctctctcc

SEQ ID NO: 198 - DNA, 22 bp, synthetic construct
  misc_feature 1..22: note = OMNI79_Serpina_S23
  source 1..22: mol_type = other DNA
gttttggagtcctgatgttata

SEQ ID NO: 199 - DNA, 22 bp, synthetic construct
  misc_feature 1..22: note = OMNI79_Serpina_S32
  source 1..22: mol_type = other DNA
attgcaaaactgatactgattc

SEQ ID NO: 200 - DNA, 22 bp, synthetic construct
  misc_feature 1..22: note = OMNI79_Serpina_S33
  source 1..22: mol_type = other DNA
cagttttgcaatctgaaagacc

SEQ ID NO: 201 - DNA, 22 bp, synthetic construct
  misc_feature 1..22: note = OMNI79_Serpina_S34
  source 1..22: mol_type = other DNA
agttttgcaatctgaaagacct

SEQ ID NO: 202 - DNA, 22 bp, synthetic construct
  misc_feature 1..22: note = OMNI79_Serpina_S38
  source 1..22: mol_type = other DNA
acagcagcaccaccacacgttc

SEQ ID NO: 203 - DNA, 22 bp, synthetic construct
  misc_feature 1..22: note = OMNI79_Serpina_S39
  source 1..22: mol_type = other DNA
ttggttctgccagaacgtgtgg

SEQ ID NO: 204 - DNA, 25 bp, synthetic construct
  misc_feature 1..25: note = OMNI79_Serpina_S40
  source 1..25: mol_type = other DNA
tttttggttctgccagaacgtgtgg

SEQ ID NO: 205 - DNA, 22 bp, synthetic construct
  misc_feature 1..22: note = OMNI79_Serpina_S49
  source 1..22: mol_type = other DNA
acacaaaagcctcagcctttca

SEQ ID NO: 206 - DNA, 22 bp, synthetic construct
  misc_feature 1..22: note = OMNI79_Serpina_S50
  source 1..22: mol_type = other DNA
gctgaggcttttgtgttgctct

SEQ ID NO: 207 - DNA, 22 bp, synthetic construct
  misc_feature 1..22: note = OMNI79_Serpina_S51
  source 1..22: mol_type = other DNA
aggcttttgtgttgctcttggt

SEQ ID NO: 208 - DNA, 22 bp, synthetic construct
  misc_feature 1..22: note = OMNI81_EMX_S11
  source 1..22: mol_type = other DNA
gctcccatgggtctaacattca

SEQ ID NO: 209 - DNA, 22 bp, synthetic construct
  misc_feature 1..22: note = OMNI81_EMX_S12
  source 1..22: mol_type = other DNA
tacaaacggcagaagctggagg

SEQ ID NO: 210 - DNA, 22 bp, synthetic construct
  misc_feature 1..22: note = OMNI81_B2M_S8
  source 1..22: mol_type = other DNA
cggcccgaatgctgtcagcttc

SEQ ID NO: 211 - DNA, 22 bp, synthetic construct
  misc_feature 1..22: note = OMNI81_B2M_S9
  source 1..22: mol_type = other DNA
actacactgaattcacccccac

SEQ ID NO: 212 - DNA, 14 bp, synthetic construct
  misc_feature 1..14: note = OMNI79_SAMD9_rs070_1 3' (PAM containing) genomic seq
  source 1..14: mol_type = other DNA
tggtgtggttttct

SEQ ID NO: 213 - DNA, 19 bp, synthetic construct
  misc_feature 1..19: note = OMNI79_SAMD9_rs070_2 3' (PAM containing) genomic seq
  source 1..19: mol_type = other DNA
tggttttctgttctacata

SEQ ID NO: 214 - DNA, 19 bp, synthetic construct
  misc_feature 1..19: note = OMNI79_SAMD9_rs201_alt 3' (PAM containing) genomic seq
  source 1..19: mol_type = other DNA
tggcattcagagaactgtg

SEQ ID NO: 215 - DNA, 19 bp, synthetic construct
  misc_feature 1..19: note = OMNI79_SAMD9_rs499_1 3' (PAM containing) genomic seq
  source 1..19: mol_type = other DNA
aggacagtaatagaaatac

SEQ ID NO: 216 - DNA, 18 bp, synthetic construct
  misc_feature 1..18: note = OMNI79_SAMD9_rs499_2 3' (PAM containing) genomic seq
  source 1..18: mol_type = other DNA
tggatgacccaagagatc

SEQ ID NO: 217 - DNA, 18 bp, synthetic construct
  misc_feature 1..18: note = OMNI79_SAMD9L_rs532_1 3' (PAM containing) genomic seq
  source 1..18: mol_type = other DNA
tggtcaatgaaggcagcc

SEQ ID NO: 218 - DNA, 18 bp, synthetic construct
  misc_feature 1..18: note = OMNI79_SAMD9L_rs532_2 3' (PAM containing) genomic seq
  source 1..18: mol_type = other DNA
aggcagccttactggtga

SEQ ID NO: 219 - DNA, 17 bp, synthetic construct
  misc_feature 1..17: note = OMNI79_Serpina_S12 3' (PAM containing) genomic seq
  source 1..17: mol_type = other DNA
aggtgcctatgatgaag

SEQ ID NO: 220 - DNA, 20 bp, synthetic construct
  misc_feature 1..20: note = OMNI79_Serpina_S13 3' (PAM containing) genomic seq
  source 1..20: mol_type = other DNA
tggaagtcctcttcctcggt

SEQ ID NO: 221 - DNA, 21 bp, synthetic construct
  misc_feature 1..21: note = OMNI79_Serpina_S14 3' (PAM containing) genomic seq
  source 1..21: mol_type = other DNA
tggtccacgtggaagtcctct

SEQ ID NO: 222 - DNA, 14 bp, synthetic construct
  misc_feature 1..14: note = OMNI79_Serpina_S22 3' (PAM containing) genomic seq
  source 1..14: mol_type = other DNA
tggcccttctatca

SEQ ID NO: 223 - DNA, 17 bp, synthetic construct
  misc_feature 1..17: note = OMNI79_Serpina_S23 3' (PAM containing) genomic seq
  source 1..17: mol_type = other DNA
aggaacagcttgggagg

SEQ ID NO: 224 - DNA, 19 bp, synthetic construct
  misc_feature 1..19: note = OMNI79_Serpina_S32 3' (PAM containing) genomic seq
  source 1..19: mol_type = other DNA
tgggacactagagtcgtgt

SEQ ID NO: 225 - DNA, 18 bp, synthetic construct
  misc_feature 1..18: note = OMNI79_Serpina_S33 3' (PAM containing) genomic seq
  source 1..18: mol_type = other DNA
tgggttcaaatcctgcct

SEQ ID NO: 226 - DNA, 18 bp, synthetic construct
  misc_feature 1..18: note = OMNI79_Serpina_S34 3' (PAM containing) genomic seq
  source 1..18: mol_type = other DNA
gggttcaaatcctgcctc

SEQ ID NO: 227 - DNA, 20 bp, synthetic construct
  misc_feature 1..20: note = OMNI79_Serpina_S38 3' (PAM containing) genomic seq
  source 1..20: mol_type = other DNA
tggcagaaccaaaaaggaac

SEQ ID NO: 228 - DNA, 23 bp, synthetic construct
  misc_feature 1..23: note = OMNI79_Serpina_S39 3' (PAM containing) genomic seq
  source 1..23: mol_type = other DNA
tggtgctgctgtccctgccctgg

SEQ ID NO: 229 - DNA, 20 bp, synthetic construct
  misc_feature 1..20: note = OMNI79_Serpina_S40 3' (PAM containing) genomic seq
  source 1..20: mol_type = other DNA
tggtggtgctgctgtccctg

SEQ ID NO: 230 - DNA, 20 bp, synthetic construct
  misc_feature 1..20: note = OMNI79_Serpina_S49 3' (PAM containing) genomic seq
  source 1..20: mol_type = other DNA
aggagcacttggggtgtttg

SEQ ID NO: 231 - DNA, 20 bp, synthetic construct
  misc_feature 1..20: note = OMNI79_Serpina_S50 3' (PAM containing) genomic seq
  source 1..20: mol_type = other DNA
tggttggattattcacaggc

SEQ ID NO: 232 - DNA, 16 bp, synthetic construct
  misc_feature 1..16: note = OMNI79_Serpina_S51 3' (PAM containing) genomic seq
  source 1..16: mol_type = other DNA
tggattattcacaggc

SEQ ID NO: 233 - RNA, 19 nt, synthetic construct
  misc_feature 1..19: note = ELANE g35 19nt
  source 1..19: mol_type = other RNA
gtccgggctgggagcgggt

SEQ ID NO: 234 - RNA, 20 nt, synthetic construct
  misc_feature 1..20: note = ELANE g35 20nt
  source 1..20: mol_type = other RNA
agtccgggctgggagcgggt

SEQ ID NO: 235 - RNA, 21 nt, synthetic construct
  misc_feature 1..21: note = ELANE g35 21nt
  source 1..21: mol_type = other RNA
cagtccgggctgggagcgggt

SEQ ID NO: 236 - RNA, 22 nt, synthetic construct
  misc_feature 1..22: note = ELANE g35 22nt
  source 1..22: mol_type = other RNA
gcagtccgggctgggagcgggt

SEQ ID NO: 237 - RNA, 23 nt, synthetic construct
  misc_feature 1..23: note = ELANE g35 23nt
  source 1..23: mol_type = other RNA
tgcagtccgggctgggagcgggt

SEQ ID NO: 238 - RNA, 24 nt, synthetic construct
  misc_feature 1..24: note = ELANE g35 24nt
  source 1..24: mol_type = other RNA
ctgcagtccgggctgggagcgggt

SEQ ID NO: 239 - RNA, 25 nt, synthetic construct
  misc_feature 1..25: note = ELANE g35 25nt
  source 1..25: mol_type = other RNA
gctgcagtccgggctgggagcgggt

SEQ ID NO: 240 - RNA, 26 nt, synthetic construct
  misc_feature 1..26: note = ELANE g35 26nt
  source 1..26: mol_type = other RNA
tgctgcagtccgggctgggagcgggt

SEQ ID NO: 241 - RNA, 80 nt, synthetic construct; identical to SEQ ID NO: 70
  misc_feature 1..80: note = OMNI-79 scaffold sequence
  source 1..80: mol_type = other RNA

SEQ ID NO: 242 - DNA, 22 bp, synthetic construct
  misc_feature 1..22: note = T1 sequence
  source 1..22: mol_type = other DNA
ggtgcggttcaccagggtgtcg

SEQ ID NO: 243 - DNA, 22 bp, synthetic construct
  misc_feature 1..22: note = T2 sequence
  source 1..22: mol_type = other DNA
ggaagagcagagccttggtctc